SEQUENCE LISTING (143 sequences)

SEQ ID NO: 1 - protein, 141 aa, Mus musculus
  REGION 1..141: note = Protein sequence of antibody 2-7 VH
MYLGLNCVFIVFLLKGVQSEVKLEESGGGLVPPGGSMKLSCVASGFTFSNYFMNWVRQSPEKGLEWVAEIRLKFTNYATHYAESVEGRFTISRDDSKSSVYLQMNNLRAEDTGIYYCTRDYGSRNGYFDYWGQGTTLTVSS

SEQ ID NO: 2 - protein, 10 aa, Mus musculus
  REGION 1..10: note = 2-7 HC CDR-1
GFTFSNYFMN

SEQ ID NO: 3 - protein, 19 aa, Mus musculus
  REGION 1..19: note = 2-7 HC CDR-2
EIRLKFTNYATHYAESVEG

SEQ ID NO: 4 - protein, 11 aa, Mus musculus
  REGION 1..11: note = 2-7 HC CDR-3
DYGSRNGYFDY

SEQ ID NO: 5 - DNA, 423 bp, Mus musculus
  misc_feature 1..423: note = Nucleotide sequence of antibody 2-7 VH
  source 1..423: mol_type = other DNA
atgtacttgggactgaactgtgtattcatagtttttctcttaaaaggtgtccagagtgaagtgaagcttgaggagtctggaggaggcttggtgccacctggaggatccatgaaactctcctgtgttgcctctggattcactttcagtaactactttatgaactgggtccgccagtctccagagaaggggcttgagtgggttgctgaaattagattgaaatttactaattatgcaacacattatgcggagtctgtggaagggaggttcaccatctcaagagatgattccaaaagtagtgtctacctccaaatgaacaacttaagagctgaagacactggcatttattactgtaccagggactacggtagtaggaatgggtactttgactactggggccaaggcaccactctcacagtctcctca

SEQ ID NO: 6 - protein, 132 aa, Mus musculus
  REGION 1..132: note = Protein sequence of antibody 2-7 VL
MRCLAEFLGLLVLWIPGAIGDIVMTQAAPSVPVTPGESVSISCRSSKSLLHSNGNTFLYWFLQRPGQSPQLLIYRMSNLASGVPDRFSGSGSGTAFTLRVSRVEAEDVGIYYCMQHLEYPYTFGGGTKLEIK

SEQ ID NO: 7 - protein, 16 aa, Mus musculus
  REGION 1..16: note = 2-7 LC CDR-1
RSSKSLLHSNGNTFLY

SEQ ID NO: 8 - protein, 7 aa, Mus musculus
  REGION 1..7: note = 2-7 LC CDR-2
RMSNLAS

SEQ ID NO: 9 - protein, 9 aa, Mus musculus
  REGION 1..9: note = 2-7 LC CDR-3
MQHLEYPYT

SEQ ID NO: 10 - DNA, 396 bp, Mus musculus
  misc_feature 1..396: note = Nucleotide sequence of antibody 2-7 VL
  source 1..396: mol_type = other DNA
atgaggtgcctagctgagttcctggggctgcttgtgctctggatccctggagccattggggatattgtgatgactcaggctgcaccctctgtacctgtcactcctggagagtcagtatccatctcctgcaggtctagtaagagtctcctgcatagtaacggcaacactttcttgtattggttcctacagaggccaggccagtctcctcagctcctgatatatcggatgtccaaccttgcctcaggagtcccagacaggttcagtggcagtgggtcaggaactgctttcacactgagagtcagtagagtggaggctgaggatgtgggtatttattactgtatgcaacatctagaatatccgtacacgttcggaggggggaccaagctggaaataaaa

SEQ ID NO: 11 - protein, 135 aa, Mus musculus
  REGION 1..135: note = Protein sequence of antibody 2-8 VH
MKCSWVIFFLMAVVTGVNSEVQLQQSGAELVRPGALVKLSCKASGFNIKEYYMHWVKQRPEQGLEWIGWIDPENGDTIYDPKFQGKASITADTSSNTAYLQLSSLTSEDTAVYYCARSRLFFAYWGQGTLVTVSA

SEQ ID NO: 12 - protein, 10 aa, Mus musculus
  REGION 1..10: note = 2-8 HC CDR-1
GFNIKEYYMH

SEQ ID NO: 13 - protein, 17 aa, Mus musculus
  REGION 1..17: note = 2-8 HC CDR-2
WIDPENGDTIYDPKFQG

SEQ ID NO: 14 - protein, 7 aa, Mus musculus
  REGION 1..7: note = 2-8 HC CDR-3
SRLFFAY

SEQ ID NO: 15 - DNA, 405 bp, Mus musculus
  misc_feature 1..405: note = Nucleotide sequence of antibody 2-8 VH
  source 1..405: mol_type = other DNA
atgaaatgcagctgggtcatcttcttcctgatggcagtggttacaggggtcaattcagaggttcagctgcagcagtctggggctgaacttgtgaggccaggggccttagtcaagttgtcctgcaaagcttctggcttcaacattaaagagtattatatgcattgggtgaagcagaggcctgaacagggcctggagtggattggatggattgatcctgagaatggtgatactatatatgacccgaagttccagggcaaggccagtataacagcagacacatcctccaacacagcctacctgcaactcagcagcctgacatctgaggacactgccgtctattactgtgctagatcgagactattctttgcttactggggccaagggactctggtcactgtctctgca

SEQ ID NO: 16 - protein, 127 aa, Mus musculus
  REGION 1..127: note = Protein sequence of antibody 2-8 VL
MESQIQVLVFVFLWLSGVDGDIVMTQSHKFMSTSVGDRVSFTCKASQDVRTAVDWYQQKPGQSPKLLIYSASYRYTGVPDRFTGTGSGTDFTFTISSVQAEDLAVYFCQQQYTTPYTFGGGTKLEIK

SEQ ID NO: 17 - protein, 11 aa, Mus musculus
  REGION 1..11: note = 2-8 LC CDR-1
KASQDVRTAVD

SEQ ID NO: 18 - protein, 7 aa, Mus musculus
  REGION 1..7: note = 2-8 LC CDR-2
SASYRYT

SEQ ID NO: 19 - protein, 9 aa, Mus musculus
  REGION 1..9: note = 2-8 LC CDR-3
QQQYTTPYT

SEQ ID NO: 20 - DNA, 381 bp, Mus musculus
  misc_feature 1..381: note = Nucleotide sequence of antibody 2-8 VL
  source 1..381: mol_type = other DNA
atggagtcacagattcaggtccttgtattcgtgtttctctggttgtctggtgttgacggagacattgtaatgacccagtctcacaaattcatgtccacatcagtaggagacagggtcagcttcacctgcaaggccagtcaggatgtgcgcactgctgtagactggtatcaacagaaaccaggacaatctcctaaactactgatttactcggcatcctaccggtacactggagtccctgatcgcttcactggcactggatctgggacggatttcactttcaccatcagcagtgtgcaggctgaagacctggcagtttacttctgtcagcaacaatatactactccgtacacgttcggaggggggaccaagctggaaataaaa

SEQ ID NO: 21 - protein, 146 aa, synthetic construct
  REGION 1..146: note = Description of Artificial Sequence: Synthetic polypeptide
  REGION 1..146: note = Protein sequence of antibody hz2-7(H1L2) VH
MDPKGSLSWRILLFLSLAFELSYGEVQLLESGGGLVQPGGSLRLSCAASGFTFSNYFMNWVRQAPGKGLEWVSEIRLKFTNYATHYADSVKGRFTISRDDSKSTLYLQMNSLRAEDTAVYYCTRDYGSRNGYFDYWGQGTLVTVSS

SEQ ID NO: 22 - protein, 10 aa, synthetic construct
  REGION 1..10: note = Description of Artificial Sequence: Synthetic peptide
  REGION 1..10: note = hz2-7(H1L2) HC CDR-1
GFTFSNYFMN

SEQ ID NO: 23 - protein, 19 aa, synthetic construct
  REGION 1..19: note = Description of Artificial Sequence: Synthetic peptide
  REGION 1..19: note = hz2-7(H1L2) HC CDR-2
EIRLKFTNYATHYADSVKG

SEQ ID NO: 24 - protein, 11 aa, synthetic construct
  REGION 1..11: note = Description of Artificial Sequence: Synthetic peptide
  REGION 1..11: note = hz2-7(H1L2) HC CDR-3
DYGSRNGYFDY

SEQ ID NO: 25 - DNA, 438 bp, synthetic construct
  misc_feature 1..438: note = Description of Artificial Sequence: Synthetic polynucleotide
  misc_feature 1..438: note = Nucleotide sequence of antibody hz2-7(H1L2) VH
  source 1..438: mol_type = other DNA
atggaccccaagggcagcctgagctggagaatcctgctgttcctgagcctggccttcgagctgagctacggcgaggttcagctgctcgagagcggaggtggtctggtgcagcctggcggtagcctgaggctgagctgcgccgcaagcggctttaccttcagcaactactttatgaattgggtgaggcaggcaccgggcaagggcctggagtgggtaagcgagatcaggctgaagttcaccaattacgctacccattacgccgacagcgtgaagggccgcttcaccatcagcagggacgactctaagagcaccctgtacctgcagatgaactcactgagggccgaggacaccgccgtgtactactgcactagggactacggcagcaggaacggctacttcgactactggggccagggcactctggtcaccgtgagcagc

SEQ ID NO: 26 - protein, 132 aa, synthetic construct
  REGION 1..132: note = Description of Artificial Sequence: Synthetic polypeptide
  REGION 1..132: note = Protein sequence of antibody hz2-7(H1L2) VL
METDTLLLWVLLLWVPGSTGDIVMTQTPLSLPVTPGEPASISCRSSKSLLHSNGNTFLYWFLQKPGQSPQLLIYRMSNLASGVPDRFSGSGSGTDFTLKISRVEAEDVGVYYCMQHLEYPYTFGGGTKLEIK

SEQ ID NO: 27 - protein, 16 aa, synthetic construct
  REGION 1..16: note = Description of Artificial Sequence: Synthetic peptide
  REGION 1..16: note = hz2-7(H1L2) LC CDR-1
RSSKSLLHSNGNTFLY

SEQ ID NO: 28 - protein, 7 aa, synthetic construct
  REGION 1..7: note = Description of Artificial Sequence: Synthetic peptide
  REGION 1..7: note = hz2-7(H1L2) LC CDR-2
RMSNLAS

SEQ ID NO: 29 - protein, 9 aa, synthetic construct
  REGION 1..9: note = Description of Artificial Sequence: Synthetic peptide
  REGION 1..9: note = hz2-7(H1L2) LC CDR-3
MQHLEYPYT

SEQ ID NO: 30 - DNA, 396 bp, synthetic construct
  misc_feature 1..396: note = Description of Artificial Sequence: Synthetic polynucleotide
  misc_feature 1..396: note = Nucleotide sequence of antibody hz2-7(H1L2) VL
  source 1..396: mol_type = other DNA
atggagaccgacaccctgctgctctgggtgctgctgctctgggtgcccggctccaccggagacatcgtgatgacccagacacccctgagcctgcccgtgacccccggtgagcccgccagcatcagctgcaggtcaagcaagagcctcctgcacagcaacggcaacacctttctgtactggttcctgcagaagccgggacagagcccccagctgctgatctacaggatgagcaacctggcctctggcgtgcccgacaggttcagcggcagtggcagcggaaccgacttcaccctgaagattagcagggtggaggccgaggacgtgggcgtgtactactgtatgcagcacctggagtacccctacaccttcggcggagggaccaaactggaaatcaag

SEQ ID NO: 31 - protein, 146 aa, synthetic construct
  REGION 1..146: note = Description of Artificial Sequence: Synthetic polypeptide
  REGION 1..146: note = Protein sequence of antibody hz2-7(H1L2 G131A) VH
MDPKGSLSWRILLFLSLAFELSYGEVQLLESGGGLVQPGGSLRLSCAASGFTFSNYFMNWVRQAPGKGLEWVSEIRLKFTNYATHYADSVKGRFTISRDDSKSTLYLQMNSLRAEDTAVYYCTRDYGSRNAYFDYWGQGTLVTVSS

SEQ ID NO: 32 - protein, 10 aa, synthetic construct
  REGION 1..10: note = Description of Artificial Sequence: Synthetic peptide
  REGION 1..10: note = hz2-7(H1L2 G131A) HC CDR-1
GFTFSNYFMN

SEQ ID NO: 33 - protein, 19 aa, synthetic construct
  REGION 1..19: note = Description of Artificial Sequence: Synthetic peptide
  REGION 1..19: note = hz2-7(H1L2 G131A) HC CDR-2
EIRLKFTNYATHYADSVKG

SEQ ID NO: 34 - protein, 11 aa, synthetic construct
  REGION 1..11: note = Description of Artificial Sequence: Synthetic peptide
  REGION 1..11: note = hz2-7(H1L2 G131A) HC CDR-3
DYGSRNAYFDY

SEQ ID NO: 35 - DNA, 438 bp, synthetic construct
  misc_feature 1..438: note = Description of Artificial Sequence: Synthetic polynucleotide
  misc_feature 1..438: note = Nucleotide sequence of antibody hz2-7(H1L2 G131A) VH
  source 1..438: mol_type = other DNA
atggaccccaagggcagcctgagctggagaatcctgctgttcctgagcctggccttcgagctgagctacggcgaggttcagctgctcgagagcggaggtggtctggtgcagcctggcggtagcctgaggctgagctgcgccgcaagcggctttaccttcagcaactactttatgaattgggtgaggcaggcaccgggcaagggcctggagtgggtaagcgagatcaggctgaagttcaccaattacgctacccattacgccgacagcgtgaagggccgcttcaccatcagcagggacgactctaagagcaccctgtacctgcagatgaactcactgagggccgaggacaccgccgtgtactactgcactagggactacggcagcaggaacgcctacttcgactactggggccagggcactctggtcaccgtgagcagc

SEQ ID NO: 36 - protein, 132 aa, synthetic construct; identical to SEQ ID NO: 26
  REGION 1..132: note = Description of Artificial Sequence: Synthetic polypeptide
  REGION 1..132: note = Protein sequence of antibody hz2-7(H1L2 G131A) VL

SEQ ID NO: 37 - protein, 16 aa, synthetic construct
  REGION 1..16: note = Description of Artificial Sequence: Synthetic peptide
  REGION 1..16: note = hz2-7(H1L2 G131A) LC CDR-1
RSSKSLLHSNGNTFLY

SEQ ID NO: 38 - protein, 7 aa, synthetic construct
  REGION 1..7: note = Description of Artificial Sequence: Synthetic peptide
  REGION 1..7: note = hz2-7(H1L2 G131A) LC CDR-2
RMSNLAS

SEQ ID NO: 39 - protein, 9 aa, synthetic construct
  REGION 1..9: note = Description of Artificial Sequence: Synthetic peptide
  REGION 1..9: note = hz2-7(H1L2 G131A) LC CDR-3
MQHLEYPYT

SEQ ID NO: 40 - DNA, 396 bp, synthetic construct; identical to SEQ ID NO: 30
  misc_feature 1..396: note = Description of Artificial Sequence: Synthetic polynucleotide
  misc_feature 1..396: note = Nucleotide sequence of antibody hz2-7(H1L2 G131A) VL
  source 1..396: mol_type = other DNA

SEQ ID NO: 41 - protein, 140 aa, synthetic construct
  REGION 1..140: note = Description of Artificial Sequence: Synthetic polypeptide
  REGION 1..140: note = Protein sequence of antibody hz2-8(H1L2) VH
MDPKGSLSWRILLFLSLAFELSYGQVQLVQSGAEVKKPGASVKVSCKASGFNIKEYYMHWVRQAPEQGLEWMGWIDPENGDTIYAQKFQGRVTITADTSTNTAYMELSSLRSEDTAVYYCARSRLFFAYWGQGTLVTVSS

SEQ ID NO: 42 - protein, 10 aa, synthetic construct
  REGION 1..10: note = Description of Artificial Sequence: Synthetic peptide
  REGION 1..10: note = hz2-8(H1L2) HC CDR-1
GFNIKEYYMH

SEQ ID NO: 43 - protein, 17 aa, synthetic construct
  REGION 1..17: note = Description of Artificial Sequence: Synthetic peptide
  REGION 1..17: note = hz2-8(H1L2) HC CDR-2
WIDPENGDTIYAQKFQG

SEQ ID NO: 44 - protein, 7 aa, synthetic construct
  REGION 1..7: note = Description of Artificial Sequence: Synthetic peptide
  REGION 1..7: note = hz2-8(H1L2) HC CDR-3
SRLFFAY

SEQ ID NO: 45 - DNA, 420 bp, synthetic construct
  misc_feature 1..420: note = Description of Artificial Sequence: Synthetic polynucleotide
  misc_feature 1..420: note = Nucleotide sequence of antibody hz2-8(H1L2) VH
  source 1..420: mol_type = other DNA
atggaccccaagggcagcctgagctggagaatcctgctgttcctgagcctggccttcgagctgagctacggccaggtgcagctggtgcagagtggggccgaggtgaagaagccgggagccagcgtgaaggtgagctgcaaggccagcggcttcaacatcaaggagtactacatgcactgggtgaggcaagcgccggaacagggcctggagtggatgggctggatcgaccccgagaacggcgataccatctacgcccagaagttccagggcagggtgaccatcaccgctgacaccagcaccaacaccgcctacatggagctgagcagcctgagaagcgaggacacagccgtgtactactgcgccaggtcacgcttgttcttcgcctactggggccaaggcaccctggtgaccgtgagctct

SEQ ID NO: 46 - protein, 127 aa, synthetic construct
  REGION 1..127: note = Description of Artificial Sequence: Synthetic polypeptide
  REGION 1..127: note = Protein sequence of antibody hz2-8(H1L2) VL
METDTLLLWVLLLWVPGSTGDIQMTQSPKSLSASVGDRVTITCRASQDVRTALDWYQQKPGQSPKLLIYSASYRYSGVPSRFSGSGSGTDFTLTISSLQPEDFATYYCQQQYTTPYTFGQGTKLEIK

SEQ ID NO: 47 - protein, 11 aa, synthetic construct
  REGION 1..11: note = Description of Artificial Sequence: Synthetic peptide
  REGION 1..11: note = hz2-8(H1L2) LC CDR-1
RASQDVRTALD

SEQ ID NO: 48 - protein, 7 aa, synthetic construct
  REGION 1..7: note = Description of Artificial Sequence: Synthetic peptide
  REGION 1..7: note = hz2-8(H1L2) LC CDR-2
SASYRYS

SEQ ID NO: 49 - protein, 9 aa, synthetic construct
  REGION 1..9: note = Description of Artificial Sequence: Synthetic peptide
  REGION 1..9: note = hz2-8(H1L2) LC CDR-3
QQQYTTPYT

SEQ ID NO: 50 - DNA, 381 bp, synthetic construct
  misc_feature 1..381: note = Description of Artificial Sequence: Synthetic polynucleotide
  misc_feature 1..381: note = Nucleotide sequence of antibody hz2-8(H1L2) VL
  source 1..381: mol_type = other DNA
atggagaccgacaccctgctgctctgggtgctgctgctctgggtgcccggctccaccggagacatccagatgacccaaagcccaaagagcctgagcgccagcgtgggcgacagggtgacgatcacctgcagagccagccaggacgtgaggaccgccctggattggtatcagcagaagcccggtcagagccccaagctcctgatctacagcgcaagctacaggtacagcggcgtgcccagcaggttcagcggctccggcagcggcaccgacttcactcttaccatcagctcactgcagcccgaggacttcgccacctactactgccagcagcagtacaccaccccctacaccttcggccagggcaccaagttggagatcaag

SEQ ID NO: 51 - protein, 140 aa, synthetic construct
  REGION 1..140: note = Description of Artificial Sequence: Synthetic polypeptide
  REGION 1..140: note = Protein sequence of antibody hz2-8 (H1L2 G80A/T82A)
MDPKGSLSWRILLFLSLAFELSYGQVQLVQSGAEVKKPGASVKVSCKASGFNIKEYYMHWVRQAPEQGLEWMGWIDPENADAIYAQKFQGRVTITADTSTNTAYMELSSLRSEDTAVYYCARSRLFFAYWGQGTLVTVSS

SEQ ID NO: 52 - protein, 10 aa, synthetic construct
  REGION 1..10: note = Description of Artificial Sequence: Synthetic peptide
  REGION 1..10: note = hz2-8 (H1L2 G80A/T82A) HC CDR-1
GFNIKEYYMH

SEQ ID NO: 53 - protein, 17 aa, synthetic construct
  REGION 1..17: note = Description of Artificial Sequence: Synthetic peptide
  REGION 1..17: note = hz2-8 (H1L2 G80A/T82A) HC CDR-2
WIDPENADAIYAQKFQG

SEQ ID NO: 54 - protein, 7 aa, synthetic construct
  REGION 1..7: note = Description of Artificial Sequence: Synthetic peptide
  REGION 1..7: note = hz2-8 (H1L2 G80A/T82A) HC CDR-3
SRLFFAY

SEQ ID NO: 55 - DNA, 420 bp, synthetic construct
  misc_feature 1..420: note = Description of Artificial Sequence: Synthetic polynucleotide
  misc_feature 1..420: note = Nucleotide sequence of antibody hz2-8 (H1L2 G80A/T82A) VH
  source 1..420: mol_type = other DNA
atggaccctaagggctctctgtcttggagaatcctgctgttcctgagcctggccttcgagctgtcttatggccaggtgcagctggtgcagtctggcgccgaagtgaaaaagcctggcgcctctgtgaaggtgtcctgcaaggcctctggcttcaacatcaaagaatattacatgcactgggtccgacaggcccctgagcaaggattggaatggatgggctggatcgaccccgagaacgccgatgccatctacgcccagaaattccagggcagagtgaccatcaccgccgacacctctaccaacaccgcctacatggaactgtccagcctgagatctgaggacaccgccgtgtactactgcgcccggtccagactgttcttcgcctattggggccagggcaccctggtcacagtttcttcc

SEQ ID NO: 56 - protein, 127 aa, synthetic construct; identical to SEQ ID NO: 46
  REGION 1..127: note = Description of Artificial Sequence: Synthetic polypeptide
  REGION 1..127: note = Protein sequence of antibody hz2-8 (H1L2 G80A/T82A) VL

SEQ ID NO: 57 - protein, 11 aa, synthetic construct
  REGION 1..11: note = Description of Artificial Sequence: Synthetic peptide
  REGION 1..11: note = hz2-8 (H1L2 G80A/T82A) LC CDR-1
RASQDVRTALD

SEQ ID NO: 58 - protein, 7 aa, synthetic construct
  REGION 1..7: note = Description of Artificial Sequence: Synthetic peptide
  REGION 1..7: note = hz2-8 (H1L2 G80A/T82A) LC CDR-2
SASYRYS

SEQ ID NO: 59 - protein, 9 aa, synthetic construct
  REGION 1..9: note = Description of Artificial Sequence: Synthetic peptide
  REGION 1..9: note = hz2-8 (H1L2 G80A/T82A) LC CDR-3
QQQYTTPYT

SEQ ID NO: 60 - DNA, 381 bp, synthetic construct
  misc_feature 1..381: note = Description of Artificial Sequence: Synthetic polynucleotide
  misc_feature 1..381: note = Nucleotide sequence of antibody hz2-8 (H1L2 G80A/T82A) VL
  source 1..381: mol_type = other DNA
atggaaaccgacacactgctgctgtgggtgctgctcttgtgggtgccaggatctaccggcgacatccagatgacccagtctccaaagtctctgtccgcctccgtgggcgacagagtgaccatcacctgtagagcctctcaggacgtgcggaccgctctggattggtatcagcagaagcctggccagtctcctaagctgctgatctactccgcctcctaccggtactctggcgtgccctccagattctccggctctggctctggcaccgactttaccctgacaatctccagcctgcagcctgaggacttcgccacctactactgccagcaacagtacaccacaccttacaccttcggccagggcaccaagctggaaatcaag

SEQ ID NO: 61 - protein, 140 aa, synthetic construct
  REGION 1..140: note = Description of Artificial Sequence: Synthetic polypeptide
  REGION 1..140: note = Protein sequence of antibody hz2-8 (H1L2 G80A) VH
MDPKGSLSWRILLFLSLAFELSYGQVQLVQSGAEVKKPGASVKVSCKASGFNIKEYYMHWVRQAPEQGLEWMGWIDPENADTIYAQKFQGRVTITADTSTNTAYMELSSLRSEDTAVYYCARSRLFFAYWGQGTLVTVSS

SEQ ID NO: 62 - protein, 10 aa, synthetic construct
  REGION 1..10: note = Description of Artificial Sequence: Synthetic peptide
  REGION 1..10: note = hz2-8 (H1L2 G80A) HC CDR-1
GFNIKEYYMH

SEQ ID NO: 63 - protein, 17 aa, synthetic construct
  REGION 1..17: note = Description of Artificial Sequence: Synthetic peptide
  REGION 1..17: note = hz2-8 (H1L2 G80A) HC CDR-2
WIDPENADTIYAQKFQG

SEQ ID NO: 64 - protein, 7 aa, synthetic construct
  REGION 1..7: note = Description of Artificial Sequence: Synthetic peptide
  REGION 1..7: note = hz2-8 (H1L2 G80A) HC CDR-3
SRLFFAY

SEQ ID NO: 65 - DNA, 420 bp, synthetic construct
  misc_feature 1..420: note = Description of Artificial Sequence: Synthetic polynucleotide
  misc_feature 1..420: note = Nucleotide sequence of antibody hz2-8 (H1L2 G80A) VH
  source 1..420: mol_type = other DNA
atggaccctaagggctctctgtcttggagaatcctgctgttcctgagcctggccttcgagctgtcttatggccaggtgcagctggtgcagtctggcgccgaagtgaaaaagcctggcgcctctgtgaaggtgtcctgcaaggcctctggcttcaacatcaaagaatattacatgcactgggtccgacaggcccctgagcaaggattggaatggatgggctggatcgaccccgagaacgccgataccatctacgcccagaaattccagggcagagtgaccatcaccgccgacacctctaccaacaccgcctacatggaactgtccagcctgagatctgaggacaccgccgtgtactactgcgcccggtccagactgttcttcgcctattggggccagggcaccctggtcacagtttcttcc

SEQ ID NO: 66 - protein, 127 aa, synthetic construct; identical to SEQ ID NO: 46
  REGION 1..127: note = Description of Artificial Sequence: Synthetic polypeptide
  REGION 1..127: note = Protein sequence of antibody hz2-8 (H1L2 G80A) VL

SEQ ID NO: 67 - protein, 11 aa, synthetic construct
  REGION 1..11: note = Description of Artificial Sequence: Synthetic peptide
  REGION 1..11: note = hz2-8 (H1L2 G80A) LC CDR-1
RASQDVRTALD

SEQ ID NO: 68 - protein, 7 aa, synthetic construct
  REGION 1..7: note = Description of Artificial Sequence: Synthetic peptide
  REGION 1..7: note = hz2-8 (H1L2 G80A) LC CDR-2
SASYRYS

SEQ ID NO: 69 - protein, 9 aa, synthetic construct
  REGION 1..9: note = Description of Artificial Sequence: Synthetic peptide
  REGION 1..9: note = hz2-8 (H1L2 G80A) LC CDR-3
QQQYTTPYT

SEQ ID NO: 70 - DNA, 381 bp, synthetic construct; identical to SEQ ID NO: 60
  misc_feature 1..381: note = Description of Artificial Sequence: Synthetic polynucleotide
  misc_feature 1..381: note = Nucleotide sequence of antibody hz2-8 (H1L2 G80A) VL
  source 1..381: mol_type = other DNA

SEQ ID NO: 71 - protein, 471 aa, synthetic construct
  REGION 1..471: note = Description of Artificial Sequence: Synthetic polypeptide
  REGION 1..471: note = Full length antibody 2-7 HC protein sequence
MYLGLNCVFIVFLLKGVQSEVKLEESGGGLVPPGGSMKLSCVASGFTFSNYFMNWVRQSPEKGLEWVAEIRLKFTNYATHYAESVEGRFTISRDDSKSSVYLQMNNLRAEDTGIYYCTRDYGSRNGYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1413 bp, synthetic construct
  misc_feature 1..1413: note = Description of Artificial Sequence: Synthetic polynucleotide
  misc_feature 1..1413: note = Full length antibody 2-7 HC nucleotide sequence
  source 1..1413: mol_type = other DNA
atgtacttgggactgaactgtgtattcatagtttttctcttaaaaggtgtccagagtgaagtgaagcttgaggagtctggaggaggcttggtgccacctggaggatccatgaaactctcctgtgttgcctctggattcactttcagtaactactttatgaactgggtccgccagtctccagagaaggggcttgagtgggttgctgaaattagattgaaatttactaattatgcaacacattatgcggagtctgtggaagggaggttcaccatctcaagagatgattccaaaagtagtgtctacctccaaatgaacaacttaagagctgaagacactggcatttattactgtaccagggactacggtagtaggaatgggtactttgactactggggccaaggcaccactctcacagtctcctcagccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaag

SEQ ID NO: 73 - protein, 238 aa, synthetic construct
  REGION 1..238: note = Description of Artificial Sequence: Synthetic polypeptide
  REGION 1..238: note = Full-length antibody 2-7 LC protein sequence
MRCLAEFLGLLVLWIPGAIGDIVMTQAAPSVPVTPGESVSISCRSSKSLLHSNGNTFLYWFLQRPGQSPQLLIYRMSNLASGVPDRFSGSGSGTAFTLRVSRVEAEDVGIYYCMQHLEYPYTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 714 bp, synthetic construct
  misc_feature 1..714: note = Description of Artificial Sequence: Synthetic polynucleotide
  misc_feature 1..714: note = Full-length antibody 2-7 LC nucleotide sequence
  source 1..714: mol_type = other DNA
atgaggtgcctagctgagttcctggggctgcttgtgctctggatccctggagccattggggatattgtgatgactcaggctgcaccctctgtacctgtcactcctggagagtcagtatccatctcctgcaggtctagtaagagtctcctgcatagtaacggcaacactttcttgtattggttcctacagaggccaggccagtctcctcagctcctgatatatcggatgtccaaccttgcctcaggagtcccagacaggttcagtggcagtgggtcaggaactgctttcacactgagagtcagtagagtggaggctgaggatgtgggtatttattactgtatgcaacatctagaatatccgtacacgttcggaggggggaccaagctggaaataaaaacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 75 - protein, 465 aa, synthetic construct
  REGION 1..465: note = Description of Artificial Sequence: Synthetic polypeptide
  REGION 1..465: note = Full length antibody 2-8 HC protein sequence
MKCSWVIFFLMAVVTGVNSEVQLQQSGAELVRPGALVKLSCKASGFNIKEYYMHWVKQRPEQGLEWIGWIDPENGDTIYDPKFQGKASITADTSSNTAYLQLSSLTSEDTAVYYCARSRLFFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - DNA, 1395 bp, synthetic construct
  misc_feature 1..1395: note = Description of Artificial Sequence: Synthetic polynucleotide
  misc_feature 1..1395: note = Full length antibody 2-8 HC nucleotide sequence
  source 1..1395: mol_type = other DNA
atgaaatgcagctgggtcatcttcttcctgatggcagtggttacaggggtcaattcagaggttcagctgcagcagtctggggctgaacttgtgaggccaggggccttagtcaagttgtcctgcaaagcttctggcttcaacattaaagagtattatatgcattgggtgaagcagaggcctgaacagggcctggagtggattggatggattgatcctgagaatggtgatactatatatgacccgaagttccagggcaaggccagtataacagcagacacatcctccaacacagcctacctgcaactcagcagcctgacatctgaggacactgccgtctattactgtgctagatcgagactattctttgcttactggggccaagggactctggtcactgtctctgcagccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaag

SEQ ID NO: 77 - protein, 233 aa, synthetic construct
  REGION 1..233: note = Description of Artificial Sequence: Synthetic polypeptide
  REGION 1..233: note = Full-length antibody 2-8 LC protein sequence
MESQIQVLVFVFLWLSGVDGDIVMTQSHKFMSTSVGDRVSFTCKASQDVRTAVDWYQQKPGQSPKLLIYSASYRYTGVPDRFTGTGSGTDFTFTISSVQAEDLAVYFCQQQYTTPYTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - DNA, 699 bp, synthetic construct
  misc_feature 1..699: note = Description of Artificial Sequence: Synthetic polynucleotide
  misc_feature 1..699: note = Full-length antibody 2-8 LC nucleotide sequence
  source 1..699: mol_type = other DNA
atggagtcacagattcaggtccttgtattcgtgtttctctggttgtctggtgttgacggagacattgtaatgacccagtctcacaaattcatgtccacatcagtaggagacagggtcagcttcacctgcaaggccagtcaggatgtgcgcactgctgtagactggtatcaacagaaaccaggacaatctcctaaactactgatttactcggcatcctaccggtacactggagtccctgatcgcttcactggcactggatctgggacggatttcactttcaccatcagcagtgtgcaggctgaagacctggcagtttacttctgtcagcaacaatatactactccgtacacgttcggaggggggaccaagctggaaataaaaacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 79 - protein, 475 aa, synthetic construct
  REGION 1..475: note = Description of Artificial Sequence: Synthetic polypeptide
  REGION 1..475: note = Full length antibody hz2-7(H1L2) HC protein sequence
MDPKGSLSWRILLFLSLAFELSYGEVQLLESGGGLVQPGGSLRLSCAASGFTFSNYFMNWVRQAPGKGLEWVSEIRLKFTNYATHYADSVKGRFTISRDDSKSTLYLQMNSLRAEDTAVYYCTRDYGSRNGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - DNA, 1428 bp, synthetic construct
  misc_feature 1..1428: note = Description of Artificial Sequence: Synthetic polynucleotide
  misc_feature 1..1428: note = Full length antibody hz2-7(H1L2) HC nucleotide sequence
  source 1..1428: mol_type = other DNA
atggaccccaagggcagcctgagctggagaatcctgctgttcctgagcctggccttcgagctgagctacggcgaggttcagctgctcgagagcggaggtggtctggtgcagcctggcggtagcctgaggctgagctgcgccgcaagcggctttaccttcagcaactactttatgaattgggtgaggcaggcaccgggcaagggcctggagtgggtaagcgagatcaggctgaagttcaccaattacgctacccattacgccgacagcgtgaagggccgcttcaccatcagcagggacgactctaagagcaccctgtacctgcagatgaactcactgagggccgaggacaccgccgtgtactactgcactagggactacggcagcaggaacggctacttcgactactggggccagggcactctggtcaccgtgagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 81 - protein, 239 aa, synthetic construct
  REGION 1..239: note = Description of Artificial Sequence: Synthetic polypeptide
  REGION 1..239: note = Full-length antibody hz2-7(H1L2) LC protein sequence
METDTLLLWVLLLWVPGSTGDIVMTQTPLSLPVTPGEPASISCRSSKSLLHSNGNTFLYWFLQKPGQSPQLLIYRMSNLASGVPDRFSGSGSGTDFTLKISRVEAEDVGVYYCMQHLEYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - DNA, 720 bp, synthetic construct
  misc_feature 1..720: note = Description of Artificial Sequence: Synthetic polynucleotide
  misc_feature 1..720: note = Full-length antibody hz2-7(H1L2) LC nucleotide sequence
  source 1..720: mol_type = other DNA
atggagaccgacaccctgctgctctgggtgctgctgctctgggtgcccggctccaccggagacatcgtgatgacccagacacccctgagcctgcccgtgacccccggtgagcccgccagcatcagctgcaggtcaagcaagagcctcctgcacagcaacggcaacacctttctgtactggttcctgcagaagccgggacagagcccccagctgctgatctacaggatgagcaacctggcctctggcgtgcccgacaggttcagcggcagtggcagcggaaccgacttcaccctgaagattagcagggtggaggccgaggacgtgggcgtgtactactgtatgcagcacctggagtacccctacaccttcggcggagggaccaaactggaaatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 83 - protein, 475 aa, synthetic construct
  REGION 1..475: note = Description of Artificial Sequence: Synthetic polypeptide
  REGION 1..475: note = Full length antibody hz2-7(H1L2 G131A) HC protein sequence
MDPKGSLSWRILLFLSLAFELSYGEVQLLESGGGLVQPGGSLRLSCAASGFTFSNYFMNWVRQAPGKGLEWVSEIRLKFTNYATHYADSVKGRFTISRDDSKSTLYLQMNSLRAEDTAVYYCTRDYGSRNAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - DNA, 1428 bp, synthetic construct
  misc_feature 1..1428: note = Description of Artificial Sequence: Synthetic polynucleotide
  misc_feature 1..1428: note = Full length antibody hz2-7(H1L2 G131A) HC nucleotide sequence
  source 1..1428: mol_type = other DNA
atggaccccaagggcagcctgagctggagaatcctgctgttcctgagcctggccttcgagctgagctacggcgaggttcagctgctcgagagcggaggtggtctggtgcagcctggcggtagcctgaggctgagctgcgccgcaagcggctttaccttcagcaactactttatgaattgggtgaggcaggcaccgggcaagggcctggagtgggtaagcgagatcaggctgaagttcaccaattacgctacccattacgccgacagcgtgaagggccgcttcaccatcagcagggacgactctaagagcaccctgtacctgcagatgaactcactgagggccgaggacaccgccgtgtactactgcactagggactacggcagcaggaacgcctacttcgactactggggccagggcactctggtcaccgtgagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 85 - protein, 239 aa, synthetic construct; identical to SEQ ID NO: 81
  REGION 1..239: note = Description of Artificial Sequence: Synthetic polypeptide
  REGION 1..239: note = Full-length antibody hz2-7(H1L2 G131A) LC protein sequence

SEQ ID NO: 86 - DNA, 720 bp, synthetic construct; identical to SEQ ID NO: 82
  misc_feature 1..720: note = Description of Artificial Sequence: Synthetic polynucleotide
  misc_feature 1..720: note = Full-length antibody hz2-7(H1L2 G131A) LC nucleotide sequence
  source 1..720: mol_type = other DNA

SEQ ID NO: 87 - protein, 469 aa, synthetic construct
  REGION 1..469: note = Description of Artificial Sequence: Synthetic polypeptide
  REGION 1..469: note = Full length antibody hz2-8(H1L2) HC protein sequence
MDPKGSLSWRILLFLSLAFELSYGQVQLVQSGAEVKKPGASVKVSCKASGFNIKEYYMHWVRQAPEQGLEWMGWIDPENGDTIYAQKFQGRVTITADTSTNTAYMELSSLRSEDTAVYYCARSRLFFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - DNA, 1410 bp, synthetic construct
  misc_feature 1..1410: note = Description of Artificial Sequence: Synthetic polynucleotide
  misc_feature 1..1410: note = Full length antibody hz2-8(H1L2) HC nucleotide sequence
  source 1..1410: mol_type = other DNA
atggaccccaagggcagcctgagctggagaatcctgctgttcctgagcctggccttcgagctgagctacggccaggtgcagctggtgcagagtggggccgaggtgaagaagccgggagccagcgtgaaggtgagctgcaaggccagcggcttcaacatcaaggagtactacatgcactgggtgaggcaagcgccggaacagggcctggagtggatgggctggatcgaccccgagaacggcgataccatctacgcccagaagttccagggcagggtgaccatcaccgctgacaccagcaccaacaccgcctacatggagctgagcagcctgagaagcgaggacacagccgtgtactactgcgccaggtcacgcttgttcttcgcctactggggccaaggcaccctggtgaccgtgagctctgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 89 - protein, 234 aa, synthetic construct
  REGION 1..234: note = Description of Artificial Sequence: Synthetic polypeptide
  REGION 1..234: note = Full-length antibody hz2-8(H1L2) LC protein sequence
METDTLLLWVLLLWVPGSTGDIQMTQSPKSLSASVGDRVTITCRASQDVRTALDWYQQKPGQSPKLLIYSASYRYSGVPSRFSGSGSGTDFTLTISSLQPEDFATYYCQQQYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - DNA, 705 bp, synthetic construct
  misc_feature 1..705: note = Description of Artificial Sequence: Synthetic polynucleotide
  misc_feature 1..705: note = Full-length antibody hz2-8(H1L2) LC nucleotide sequence
  source 1..705: mol_type = other DNA
atggagaccgacaccctgctgctctgggtgctgctgctctgggtgcccggctccaccggagacatccagatgacccaaagcccaaagagcctgagcgccagcgtgggcgacagggtgacgatcacctgcagagccagccaggacgtgaggaccgccctggattggtatcagcagaagcccggtcagagccccaagctcctgatctacagcgcaagctacaggtacagcggcgtgcccagcaggttcagcggctccggcagcggcaccgacttcactcttaccatcagctcactgcagcccgaggacttcgccacctactactgccagcagcagtacaccaccccctacaccttcggccagggcaccaagttggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 91 - protein, 469 aa, synthetic construct
  REGION 1..469: note = Description of Artificial Sequence: Synthetic polypeptide
  REGION 1..469: note = Full length antibody hz2-8 (H1L2 G80A/T82A) HC protein sequence
MDPKGSLSWRILLFLSLAFELSYGQVQLVQSGAEVKKPGASVKVSCKASGFNIKEYYMHWVRQAPEQGLEWMGWIDPENADAIYAQKFQGRVTITADTSTNTAYMELSSLRSEDTAVYYCARSRLFFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - DNA, 1410 bp, synthetic construct
  misc_feature 1..1410: note = Description of Artificial Sequence: Synthetic polynucleotide
  misc_feature 1..1410: note = Full length antibody hz2-8 (H1L2 G80A/T82A) HC nucleotide sequence
  source 1..1410: mol_type = other DNA
atggaccctaagggctctctgtcttggagaatcctgctgttcctgagcctggccttcgagctgtcttatggccaggtgcagctggtgcagtctggcgccgaagtgaaaaagcctggcgcctctgtgaaggtgtcctgcaaggcctctggcttcaacatcaaagaatattacatgcactgggtccgacaggcccctgagcaaggattggaatggatgggctggatcgaccccgagaacgccgatgccatctacgcccagaaattccagggcagagtgaccatcaccgccgacacctctaccaacaccgcctacatggaactgtccagcctgagatctgaggacaccgccgtgtactactgcgcccggtccagactgttcttcgcctattggggccagggcaccctggtcacagtttcttccgcttccaccaagggacccagcgtgttccctctggctccttccagcaagtctacctctggcggaacagctgctctgggctgcctggtcaaggactactttccagagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgacatcgccgtggaatgggagagcaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggctag

SEQ ID NO: 93 - protein, 234 aa, synthetic construct; identical to SEQ ID NO: 89
  REGION 1..234: note = Description of Artificial Sequence: Synthetic polypeptide
  REGION 1..234: note = Full-length antibody hz2-8 (H1L2 G80A/T82A) LC protein sequence

SEQ ID NO: 94 - DNA, 705 bp, synthetic construct
  misc_feature 1..705: note = Description of Artificial Sequence: Synthetic polynucleotide
  misc_feature 1..705: note = Full-length antibody hz2-8 (H1L2 G80A/T82A) LC nucleotide sequence
  source 1..705: mol_type = other DNA
atggaaaccgacacactgctgctgtgggtgctgctcttgtgggtgccaggatctaccggcgacatccagatgacccagtctccaaagtctctgtccgcctccgtgggcgacagagtgaccatcacctgtagagcctctcaggacgtgcggaccgctctggattggtatcagcagaagcctggccagtctcctaagctgctgatctactccgcctcctaccggtactctggcgtgccctccagattctccggctctggctctggcaccgactttaccctgacaatctccagcctgcagcctgaggacttcgccacctactactgccagcaacagtacaccacaccttacaccttcggccagggcaccaagctggaaatcaagagaaccgtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgacactgtccaaggccgactacgagaagcacaaggtgtacgcctgcgaagtgacccatcagggcctgtctagccctgtgaccaagtctttcaaccggggcgagtgttag

SEQ ID NO: 95 - protein, 469 aa, synthetic construct
  REGION 1..469: note = Description of Artificial Sequence: Synthetic polypeptide
  REGION 1..469: note = Full length antibody hz2-8 (H1L2 G80A) HC protein sequence
MDPKGSLSWRILLFLSLAFELSYGQVQLVQSGAEVKKPGASVKVSCKASGFNIKEYYMHWVRQAPEQGLEWMGWIDPENADTIYAQKFQGRVTITADTSTNTAYMELSSLRSEDTAVYYCARSRLFFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - DNA, 1410 bp, synthetic construct
  misc_feature 1..1410: note = Description of Artificial Sequence: Synthetic polynucleotide
  misc_feature 1..1410: note = Full length antibody hz2-8 (H1L2 G80A) HC nucleotide sequence
  source 1..1410: mol_type = other DNA
atggaccctaagggctctctgtcttggagaatcctgctgttcctgagcctggccttcgagctgtcttatggccaggtgcagctggtgcagtctggcgccgaagtgaaaaagcctggcgcctctgtgaaggtgtcctgcaaggcctctggcttcaacatcaaagaatattacatgcactgggtccgacaggcccctgagcaaggattggaatggatgggctggatcgaccccgagaacgccgataccatctacgcccagaaattccagggcagagtgaccatcaccgccgacacctctaccaacaccgcctacatggaactgtccagcctgagatctgaggacaccgccgtgtactactgcgcccggtccagactgttcttcgcctattggggccagggcaccctggtcacagtttcttccgcttccaccaagggacccagcgtgttccctctggctccttccagcaagtctacctctggcggaacagctgctctgggctgcctggtcaaggactactttccagagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgacatcgccgtggaatgggagagcaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggctag

SEQ ID NO: 97 - protein, 234 aa, synthetic construct; identical to SEQ ID NO: 89
  REGION 1..234: note = Description of Artificial Sequence: Synthetic polypeptide
  REGION 1..234: note = Full-length antibody hz2-8 (H1L2 G80A) LC protein sequence

SEQ ID NO: 98 - DNA, 705 bp, synthetic construct; identical to SEQ ID NO: 94
  misc_feature 1..705: note = Description of Artificial Sequence: Synthetic polynucleotide
  misc_feature 1..705: note = Full-length LC antibody hz2-8 (H1L2 G80A) nucleotide sequence
  source 1..705: mol_type = other DNA

SEQ ID NO: 99 - protein, 673 aa, Homo sapiens
  REGION 1..673: note = Human C1s full-length wt protein sequence (huC1s)
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 100 - DNA, 2022 bp, synthetic construct
  misc_feature 1..2022: note = Description of Artificial Sequence: Synthetic polynucleotide
  misc_feature 1..2022: note = Recombinant Human C1s nucleotide sequence (codon optimized)
  source 1..2022: mol_type = other DNA
gagcctactatgtacggcgagatcctgtctcctaactaccctcaggcttacccctccgaggtggaaaagtcctgggatatcgaggtgcccgaaggctacggcatccacctgtacttcacccacctggacatcgagctgtccgagaactgcgcctacgactccgtgcagatcatctccggcgataccgaagagggcagactgtgcggccagcggtcctctaacaatccccactctcctatcgtggaagagttccaggtgccatacaacaagctgcaagtgatcttcaagtccgacttctccaacgaggaacggttcaccggcttcgccgcttactatgtggccaccgacatcaacgagtgcaccgacttcgtggacgtgccctgctctcacttctgcaacaactttatcggcggctacttctgcagctgccctcctgagtacttcctgcacgacgacatgaagaactgcggcgtgaactgctccggcgacgtgttcacagctctgatcggagagatcgcctctccaaattaccccaagccttatcctgagaactcccgctgcgagtaccagatcagactggaaaagggcttccaggtggtggtcaccctgcggcgcgaggattttgatgtggaagccgctgattccgccggcaactgcctggattctctggtgtttgtggccggcgacagacagttcggcccttattgtggccatggcttccccggacctctgaacatcgagacaaagagcaacgccctggatatcatcttccagaccgacctgaccggccagaagaaaggctggaagctgagataccacggcgaccccatgccttgtcctaaagaggacacccctaactccgtgtgggagcccgccaaggccaaatacgtgttcagagatgtggtccagatcacctgtctggacggctttgaggtggtggaaggcagagtgggcgccacctctttctactctacctgccagtccaacggcaagtggtccaactccaagctgaagtgccagcctgtggactgcggcatccctgagtctatcgagaacggcaaggtggaagatcccgagagcaccctgttcggctccgtgatcagatatacctgcgaggaaccctactactacatggaaaacggcggaggcggcgagtatcactgtgctggcaatggctcttgggtcaacgaggtgctgggacccgaactgcctaagtgtgttcctgtgtgtggcgtgcccagagagcccttcgaggaaaagcagagaatcatcggcggcagcgacgccgatatcaagaacttcccctggcaagtgttcttcgacaacccctgggctggcggcgctctgatcaatgagtattgggtgctgaccgccgctcacgtggtcgagggaaatagagaacccactatgtatgtgggctccaccagcgtgcagacctccagactggccaagtccaagatgctgacccctgagcacgtgttcatccatcctggctggaaactgctggaagtgcccgagggcagaacaaacttcgataacgatatcgccctcgtgcggctgaaggaccctgtgaagatgggccctaccgtgtctcctatctgtctgcctggcacctcctccgactacaacctgatggatggcgacctgggcctgatctctggatggggaagaaccgagaagcgggatagagccgtcagactgaaggccgctagactgcctgttgctcctctgcggaagtgcaaagaagtgaaggtcgagaagcctaccgccgatgccgaggcctatgtgttcaccccaaacatgatctgcgctggcggagagaaaggcatggactcttgcaagggcgattccggcggagcattcgcagtgcaggaccctaacgacaagaccaagttctacgctgccggcctggtgtcttggggacctcagtgtggaacctacggcctgtacaccagagtgaagaactacgtggactggatcatgaagaccatgcaagagaacagcacccctagagaggactag

SEQ ID NO: 101 - protein, 673 aa, Mus musculus
  REGION 1..673: note = Mouse C1s protein sequence (moC1s)
EPTMHGEILSPNYPQAYPNDVVKSWDIEVPEGFGIHLYFTHVDIEPSESCAYDSVQIISGGIEEGRLCGQKTSKSPNSPIIEEFQFPYNKLQVVFTSDFSNEERFTGFAAYYTAIDINECTDFTDVPCSHFCNNFIGGYFCSCPPEYFLHDDMRNCGVNCSGDVFTALIGEISSPNYPNPYPENSRCEYQIQLQEGFQVVVTMQREDFDVEPADSEGNCPDSLTFASKNQQFGPYCGNGFPGPLTIRTQSNTLGIVFQTDLMGQKKGWKLRYHGDPISCAKKITANSTWEPDKAKYVFKDVVKITCVDGFEVVEGHVSSTSYYSTCQSDGQWSNSGLKCQPVYCGIPDPIANGKVEEPENSVFGTVVHYTCEEPYYYMEHEEGGEYRCAANGRWVNDQLGIELPRCIPACGVPTEPFQVHQRIFGGQPAKIENFPWQVFFNHPRASGALINEYWVLTAAHVLEKISDPLMYVGTMSVRTTLLENAQRLYSKRVFIHPSWKKEDDPNTRTNFDNDIALVQLKDPVKMGPKVSPICLPGTSSEYNVSPGDMGLISGWGSTEKKVFVINLRGAKVPVTSLETCKQVKEENPTVRPEDYVFTDNMICAGEKGVDSCHGDSGGAFAFQVPNVTVPKFYVAGLVSWGKRCGTYGVYTKVKNYVDWILKTMQENSGPRKD

SEQ ID NO: 102 - DNA, 2022 bp, synthetic construct
  misc_feature 1..2022: note = Description of Artificial Sequence: Synthetic polynucleotide
  misc_feature 1..2022: note = Recombinant mouse C1s nucleotide sequence (codon optimized)
  source 1..2022: mol_type = other DNA
gagcctacaatgcacggcgagatcctgtctccaaactaccctcaggcttaccccaacgacgtggtcaagtcctgggatatcgaggtgccagaaggcttcggcatccacctgtacttcacccacgtggacatcgagccttccgagtcttgcgcctacgactccgtgcagatcatctccggcggaatcgaagagggcagactgtgcggccagaaaacctccaagtctcccaactctcccattatcgaggaatttcagttcccctacaacaagctgcaggtcgtgttcacctccgacttctccaacgaggaacggttcaccggcttcgccgcttactacaccgccatcgacatcaacgagtgcaccgacttcaccgatgtgccctgcagccacttctgcaacaactttatcggcggctacttctgcagctgccctcctgagtacttcctgcacgacgacatgagaaactgcggcgtgaactgctccggcgacgtgttcacagctctgatcggagagatctcctctcctaattaccccaatccttatcctgagaactcccgctgcgagtaccagatccagctgcaagagggcttccaggtggtggtcaccatgcagcgcgaggacttcgatgtggaacctgccgattctgagggcaactgccctgactctctgaccttcgcctccaagaaccagcagttcggcccttactgtggcaacggctttcctggacctctgaccatcagaacccagagcaacaccctgggcatcgtgttccagaccgatctgatgggccagaagaaaggctggaagctgagataccacggcgaccccatcagctgcgccaagaagatcaccgccaactccacctgggagcctgacaaggccaaatacgtgttcaaggacgtcgtgaagatcacatgcgtggacggctttgaggtggtggaaggccacgtgtccagcaccagctactactctacctgccagtctgacggccagtggtccaactctggcctgaagtgtcagcctgtgtactgcggcatccccgatcctatcgccaatggcaaggtggaagaacccgagaactccgtgtttggcaccgtggtgcactatacctgcgaggaaccctactactacatggaacatgaggaaggcggcgagtaccgctgtgccgctaatggaagatgggtcaacgaccagctgggcattgagctgcccagatgcattcctgcttgtggcgtgccaaccgagcctttccaggtgcaccagagaatctttggcggccagcctgccaagatcgagaacttcccttggcaagtgttcttcaatcaccccagagcctctggcgccctgatcaatgagtactgggtgctgaccgctgctcacgtgctggaaaagatcagcgaccctcttatgtacgtgggcaccatgtccgtgcggaccacactgttggagaacgcccagcggctgtactccaagagagtgttcatccatcctagctggaagaaagaggacgaccccaacaccaggaccaacttcgacaacgatatcgccctggtgcagctgaaggaccccgtgaagatgggccctaaggtgtcccctatctgtctgcccggcacctcctctgagtacaatgtgtctcctggcgacatgggcctgatctctggctggggctctaccgagaagaaagtgttcgtgatcaacctgagaggcgccaaggtgcccgtgacctctctggaaacatgcaagcaagtgaaagaagagaaccccaccgtccggcctgaggactatgtgttcaccgacaacatgatctgtgccggcgagaaaggcgtggactcttgccacggcgattctggcggagccttcgcctttcaagtgcccaatgtgaccgtgcctaagttctacgtggccggcctggtgtcttggggcaagagatgtggtacttacggcgtgtacaccaaagtgaagaactacgtggactggattctcaagaccatgcaagagaacagcggccctcggaaggactag

SEQ ID NO: 103 - protein, 673 aa, Macaca fascicularis
  REGION 1..673: note = Cynomolgus monkey C1s protein sequence (cyC1s)
EPTMYGEILSPNYPQAYPSEVEKSWDIEVPEGYGIHLYFTHLDIELSENCAYDSVQIMSGDIEEGRLCGQRTSNNPHSPIVEEFQVPYNKLQVIFKSDFSNEERFTGFAAYYVATDINECTDFVDAPCSHFCNNFIGGYFCSCPPEYFLHDDMKNCGVNCSGDVFTALIGEIASPNYPKPYPENSRCEYQIRLEKGFQVVVTVRREDFDVEPADSEGNCLDSLVFVAGDQQFGPYCGRGFPGPLNIETKSNALDIIFQTDLTGQNKGWKLRYHGDPMPCPKEETPTSVWEPAKAKYVFRDVVRITCLDGFEVVEGRVGATSFHSTCQSNGKWSNSKLKCQPVDCGIPESIENGKVEDPESTLFGSVTRYTCEEPYYYMENGGNGQYHCASNGSWVNEALSPELPKCVPVCGVPREPFEGKQRIIGGSDADIKNFPWQVFFDNPWAGGALIDEYWVLTAAHVVEGNQEPTMYVGSTSVQTSRLAKSKMLTSERVFIHPGWKLLEVPEARTNFDNDIALVQLKDPVKMGPTVAPICLPGTSSDYNLMDGDLGLIAGWGRTEKRDRALRLKAARLPVAPLRKCREVKVENPKADAGAYVFTPNMICAGGEKGMDSCKGDSGGAFAVQDPNDKAKFYVAGLVSWGPQCGTYGLYTRVQNYVDWIKKTMQENSTPSKD

SEQ ID NO: 104 - DNA, 2022 bp, synthetic construct
  misc_feature 1..2022: note = Description of Artificial Sequence: Synthetic polynucleotide
  misc_feature 1..2022: note = Recombinant cynomolgus monkey C1s nucleotide sequence (codon optimized)
  source 1..2022: mol_type = other DNA
gagcctactatgtacggcgagatcctgtctcctaactaccctcaggcttacccctccgaggtggaaaagtcctgggatatcgaggtgcccgaaggctacggcatccacctgtacttcacccacctggacatcgagctgtccgagaactgcgcctacgactccgtgcagatcatgtccggcgatatcgaagagggcagactgtgcggccagcggacctctaacaatccccactctcctatcgtggaagagttccaggtgccatacaacaagctgcaagtgatcttcaagtccgacttctccaacgaggaacggttcaccggcttcgccgcttactatgtggccaccgacatcaacgagtgcaccgactttgtggacgccccttgcagccacttctgcaacaactttatcggcggctacttctgcagctgccctcctgagtatttcctgcacgacgacatgaagaactgcggcgtgaactgctccggcgacgtgttcacagctctgatcggagagatcgcctctccaaattaccccaagccttatcctgagaactcccgctgcgagtaccagatcagactggagaagggcttccaggtggtggtcacagtgcggcgcgaggactttgatgtggaacctgctgactccgagggcaactgcctggactctctggtgtttgtggctggcgatcagcagttcggcccttactgtggcagaggctttcccggaccactgaacatcgagacaaagagcaacgccctggatatcatcttccagaccgacctgaccggccagaacaaaggctggaagctgagataccacggcgaccccatgccttgtcctaaagaagagacacccacctccgtgtgggagcccgccaaggctaaatacgtgttcagagatgtcgtgcggatcacctgtctggacggctttgaggtggtggaaggcagagtgggcgccacctctttccactctacctgccagtctaacggcaagtggtccaactccaagctgaagtgccagcctgtggactgcggcatccctgagtctatcgagaacggcaaggtggaagatcccgagagcaccctgtttggcagcgtgaccagatatacctgcgaggaaccctactactacatggaaaacggcggcaacggccagtaccactgtgcctctaatggctcctgggtcaacgaggccctgtctccagaactgcctaagtgcgtgccagtgtgtggcgtgccaagagagccttttgagggcaagcagagaatcatcggcggcagcgacgccgatatcaagaacttcccctggcaagtgttcttcgacaacccctgggctggcggagccctgatcgatgaatattgggtgctgaccgccgctcacgtggtcgagggaaatcaagaacccactatgtatgtgggctccaccagcgtgcagacctccagactggccaagtccaagatgctgaccagcgagcgggtgttcatccatcctggctggaaactgctggaggtcccagaggccaggaccaacttcgacaatgatatcgccctggtgcagctgaaggaccccgtgaagatgggacctaccgtggctcctatctgtctgcctggcacctcctccgactacaacctgatggatggcgacctgggcctgatcgctggatggggaagaaccgagaagagagacagagccctgagactgaaggccgccagattgcctgttgctcctctgcggaagtgccgggaagtgaaggtcgagaaccctaaggctgatgccggcgcttatgtgttcacccctaatatgatctgcgctggcggcgagaaaggcatggactcttgcaaaggcgattctggcggcgcattcgcagtgcaggaccctaacgataaggccaagttctacgtggccggcctggtgtcttggggacctcagtgtggaacatacggcctgtacacacgggtgcagaactacgtggactggatcaagaaaaccatgcaagagaacagcaccccttccaaggactag

SEQ ID NO: 105 - protein, 673 aa, Rattus norvegicus
  REGION 1..673: note = Rat C1s protein sequence (raC1s)
EPTMYGEILSPNYPQAYPNEVVKTWDIEVPEGFGIHLYFTHLDMELSENCAYDSVQIISGGIEEERLCGQRTSKSPNSPTVEEFQFPYNRLQVVFTSDFSNEERFTGFAAYYSAVDVNECTDFTDVPCSHFCNNFIGGYFCSCPPEYFLHDDMRTCGVNCSGDVFTALIGEIASPNYPNPYPENSRCEYQIRLQEGFRLVLTIRREDFDVEPADSEGNCHDSLTFAAKNQQFGPYCGNGFPGPLTIKTQSNTLDIVFQTDLTGQNKGWKLRYHGDPIPCPKEISANSIWEPEKAKYVFKDVVKITCVDGFEVVEGNVGSTSFYSTCQSNGQWSNSRLECQPVDCGVPEPIENGKVEDPEDTVFGSVIHYTCEEPYYYMEQEEGGEYHCAANGSWVNDQLGVELPKCIPVCGVPTEPFKVQQRIFGGYSTKIQSFPWQVYFESPRGGGALIDEYWVLTAAHVVEGNSDPVMYVGSTLLKIERLRNAQRLITERVIIHPSWKQEDDLNTRTNFDNDIALVQLKDPVKMGPTVAPICLPETSSDYNPSEGDLGLISGWGRTENRTNVIQLRGAKLPITSLEKCQQVKVENPKARSNDYVFTDNMICAGEKGVDSCEGDSGGAFALPVPNVKDPKFYVAGLVSWGKKCGTYGIYTKVKNYVDWILKTMQENSGPKKD

SEQ ID NO: 106 - DNA, 2022 bp, synthetic construct
  misc_feature 1..2022: note = Description of Artificial Sequence: Synthetic polynucleotide
  misc_feature 1..2022: note = Recombinant rat C1s nucleotide sequence
  source 1..2022: mol_type = other DNA
gagcctaccatgtatggggagatcctgtcccctaattatccccaggcgtaccccaatgaggtcgtgaaaacttgggacatagaagtcccagaggggtttgggattcacctttacttcacccatctggacatggagctgtcagagaactgtgcgtacgactcagtgcagataatctcaggaggcatcgaggaagagagactctgtggccagaggaccagcaagagtcccaactcccccactgtagaagagtttcaattcccatacaataggctccaggtggtctttacgtcagacttctccaacgaggaacggtttactggctttgcagcgtattactcagccgtagatgtaaatgaatgcacagactttacagatgtcccttgcagccacttctgcaataacttcattggtggatacttctgctcctgccccccagaatacttcctccacgatgacatgaggacttgtggggtcaactgtagtggggatgtattcactgccctgattggggagatcgcaagtcccaattatcccaacccatacccggagaactcaaggtgtgaataccagattcggctgcaggagggcttccgactggtgttgactatccggagagaagattttgatgtggaaccagctgactcagaggggaactgccacgacagtttgacttttgctgcaaaaaaccaacagtttggtccttactgtggcaatggattccctggacctctaactattaaaacccagagcaatactcttgatattgtctttcaaactgacctaacggggcaaaataaaggctggaagcttcgttaccatggagatcccatcccctgtcccaaagaaatcagtgctaattctatctgggagcccgaaaaggcaaaatacgtgttcaaagatgtcgtgaagataacctgtgtggatggattcgaagttgtggagggaaatgttggctcaacatcattctattccacttgtcaaagcaacggacagtggagcaattccaggctagagtgtcaacctgtggactgtggtgttccagaacccattgagaatggtaaagttgaagacccagaagacactgtattcggctccgtcatccactacacgtgcgaagagccatattactacatggaacaggaagaaggcggagagtatcactgtgctgctaatgggagctgggtgaatgaccagctgggtgtcgagcttccaaaatgtattccagtctgtggagtacccaccgagccctttaaagtacagcagaggatatttggaggatactctacaaagattcaaagttttccttggcaggtctactttgagtccccccgaggtggcggggctcttatcgatgagtactgggtgctgacggccgctcacgttgtggagggaaactctgacccagtgatgtatgtcgggtccacacttctgaaaatagagcggttgagaaatgcccagaggctcatcactgaacgtgtgattattcatcccagctggaaacaagaggacgacctgaatacacggacaaattttgacaatgacattgccctggtgcagctcaaagaccctgtgaaaatgggacccactgttgcccccatctgcctgccagaaacctcctcagactacaacccctcagagggtgacctggggctgatctctgggtggggccgaacagagaatagaaccaatgttattcaactcagaggggcgaagttacccataacatctttagaaaagtgccagcaggtgaaagtggaaaacccgaaagcgaggtcaaacgactatgttttcactgacaacatgatctgtgctggggaaaagggtgtggacagctgtgaaggtgacagcggaggggcttttgctctgccggtccccaatgtcaaggaccccaaattctatgtggctggcctggtgtcctgggggaaaaagtgtgggacctatgggatctacacaaaggtaaagaactacgtggactggatcctgaaaactatgcaggagaatagtgggcccaagaaggactag

SEQ ID NO: 107 - protein, 147 aa, synthetic construct
  REGION 1..147: note = Description of Artificial Sequence: Synthetic polypeptide
  REGION 1..147: note = Human C1s deletion mutant 1 (huC1sM151) protein sequence
YHGDPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQR

SEQ ID NO: 108 - DNA, 456 bp, synthetic construct
  misc_feature 1..456: note = Description of Artificial Sequence: Synthetic polynucleotide
  misc_feature 1..456: note = Recombinant Human C1s deletion mutant 1(huC1sM151) nucleotide sequence
  source 1..456: mol_type = other DNA
taccacggcgaccccatgccttgtcctaaagaggacacccctaactccgtgtgggagcccgccaaggccaaatacgtgttcagagatgtggtccagatcacctgtctggacggctttgaggtggtggaaggcagagtgggcgccacctctttctactctacctgccagtccaacggcaagtggtccaactccaagctgaagtgccagcctgtggactgcggcatccctgagtctatcgagaacggcaaggtggaagatcccgagagcaccctgttcggctccgtgatcagatatacctgcgaggaaccctactactacatggaaaacggcggaggcggcgagtatcactgtgctggcaatggctcttgggtcaacgaggtgctgggacccgaactgcctaagtgtgttcctgtgtgtggcgtgcccagagagcccttcgaggaaaagcagagatag

SEQ ID NO: 109 - protein, 428 aa, synthetic construct
  REGION 1..428: note = Description of Artificial Sequence: Synthetic polypeptide
  REGION 1..428: note = Human C1s deletion mutant 2 (huC1sNHC) protein sequence
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRHHHHHH

SEQ ID NO: 110 - DNA, 1269 bp, synthetic construct
  misc_feature 1..1269: note = Description of Artificial Sequence: Synthetic polynucleotide
  misc_feature 1..1269: note = Recombinant Human C1s deletion mutant 2 (huC1sNHC) nucleotide sequence
  source 1..1269: mol_type = other DNA
gagcctactatgtacggcgagatcctgtctcctaactaccctcaggcttacccctccgaggtggaaaagtcctgggatatcgaggtgcccgaaggctacggcatccacctgtacttcacccacctggacatcgagctgtccgagaactgcgcctacgactccgtgcagatcatctccggcgataccgaagagggcagactgtgcggccagcggtcctctaacaatccccactctcctatcgtggaagagttccaggtgccatacaacaagctgcaagtgatcttcaagtccgacttctccaacgaggaacggttcaccggcttcgccgcttactatgtggccaccgacatcaacgagtgcaccgacttcgtggacgtgccctgctctcacttctgcaacaactttatcggcggctacttctgcagctgccctcctgagtacttcctgcacgacgacatgaagaactgcggcgtgaactgctccggcgacgtgttcacagctctgatcggagagatcgcctctccaaattaccccaagccttatcctgagaactcccgctgcgagtaccagatcagactggaaaagggcttccaggtggtggtcaccctgcggcgcgaggattttgatgtggaagccgctgattccgccggcaactgcctggattctctggtgtttgtggccggcgacagacagttcggcccttattgtggccatggcttccccggacctctgaacatcgagacaaagagcaacgccctggatatcatcttccagaccgacctgaccggccagaagaaaggctggaagctgagataccacggcgaccccatgccttgtcctaaagaggacacccctaactccgtgtgggagcccgccaaggccaaatacgtgttcagagatgtggtccagatcacctgtctggacggctttgaggtggtggaaggcagagtgggcgccacctctttctactctacctgccagtccaacggcaagtggtccaactccaagctgaagtgccagcctgtggactgcggcatccctgagtctatcgagaacggcaaggtggaagatcccgagagcaccctgttcggctccgtgatcagatatacctgcgaggaaccctactactacatggaaaacggcggaggcggcgagtatcactgtgctggcaatggctcttgggtcaacgaggtgctgggacccgaactgcctaagtgtgttcctgtgtgtggcgtgcccagagagcccttcgaggaaaagcagagatag

SEQ ID NO: 111 - protein, 389 aa, synthetic construct
  REGION 1..389: note = Description of Artificial Sequence: Synthetic polypeptide
  REGION 1..389: note = Human C1s deletion mutant 3 (NHCD33) protein sequence
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCA

SEQ ID NO: 112 - protein, 673 aa, synthetic construct
  REGION 1..673: note = Description of Artificial Sequence: Synthetic polypeptide
  REGION 1..673: note = Protein sequence of Human C1s M1(M277I)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPIPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 113 - protein, 673 aa, synthetic construct
  REGION 1..673: note = Description of Artificial Sequence: Synthetic polypeptide
  REGION 1..673: note = Protein sequence of human C1s M2 (M277I, K331Q)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPIPCPKEDTPNSVWEPAKAKYVFRDVVQITCLDGFEVVEGRVGATSFYSTCQSNGQWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 114 - protein, 673 aa, synthetic construct
  REGION 1..673: note = Description of Artificial Sequence: Synthetic polypeptide
  REGION 1..673: note = Protein sequence of human C1s M3 (M277I, P278, N329D)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPISCPKEDTPNSVWEPAKAKYVFRDVVQITCLDGFEVVEGRVGATSFYSTCQSDGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 115 - protein, 673 aa, synthetic construct
  REGION 1..673: note = Description of Artificial Sequence: Synthetic polypeptide
  REGION 1..673: note = Protein sequence of human C1s M4 (P280A, E282K)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCAKKDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 116 - protein, 673 aa, synthetic construct
  REGION 1..673: note = Description of Artificial Sequence: Synthetic polypeptide
  REGION 1..673: note = Protein sequence of human C1s M5 (K336G)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSGLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 117 - protein, 673 aa, synthetic construct
  REGION 1..673: note = Description of Artificial Sequence: Synthetic polypeptide
  REGION 1..673: note = Protein sequence of human C1s M6 (P280A, E282K, K336G)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCAKKDTPNSVWEPAKAKYVFRDVVQITCLDGFEVVEGRVGATSFYSTCQSNGKWSNSGLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 118 - protein, 673 aa, synthetic construct
  REGION 1..673: note = Description of Artificial Sequence: Synthetic polypeptide
  REGION 1..673: note = Protein sequence of human C1s M7 (D343Y, E351A)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVYCGIPESIANGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 119 - protein, 673 aa, synthetic construct
  REGION 1..673: note = Description of Artificial Sequence: Synthetic polypeptide
  REGION 1..673: note = Protein sequence of human C1s M8 (E351A)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIANGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 120 - protein, 673 aa, synthetic construct
  REGION 1..673: note = Description of Artificial Sequence: Synthetic polypeptide
  REGION 1..673: note = Protein sequence of human C1s M9 (K336G, D343Y, E351A)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSGLKCQPVYCGIPESIANGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 121 - protein, 673 aa, synthetic construct
  REGION 1..673: note = Description of Artificial Sequence: Synthetic polypeptide
  REGION 1..673: note = Protein sequence of human C1s M10 (D283I, P285A)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEITANSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 122 - protein, 673 aa, synthetic construct
  REGION 1..673: note = Description of Artificial Sequence: Synthetic polypeptide
  REGION 1..673: note = Protein sequence of human C1s M11 (S349P)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPEPIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 123 - protein, 673 aa, synthetic construct
  REGION 1..673: note = Description of Artificial Sequence: Synthetic polypeptide
  REGION 1..673: note = Protein sequence of human C1s M12 (G381E, G382E)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENEEGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 124 - protein, 673 aa, synthetic construct
  REGION 1..673: note = Description of Artificial Sequence: Synthetic polypeptide
  REGION 1..673: note = Protein sequence of human C1s M13 (A292D)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPDKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 125 - protein, 673 aa, synthetic construct
  REGION 1..673: note = Description of Artificial Sequence: Synthetic polypeptide
  REGION 1..673: note = Protein sequence of human C1s M14 (R316H)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGHVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 126 - protein, 673 aa, synthetic construct
  REGION 1..673: note = Description of Artificial Sequence: Synthetic polypeptide
  REGION 1..673: note = Protein sequence of human C1s M15 (S360N)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPENTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 127 - protein, 673 aa, synthetic construct
  REGION 1..673: note = Description of Artificial Sequence: Synthetic polypeptide
  REGION 1..673: note = Protein sequence of human C1s M16 (R368H)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIHYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 128 - protein, 673 aa, synthetic construct
  REGION 1..673: note = Description of Artificial Sequence: Synthetic polypeptide
  REGION 1..673: note = Protein sequence of human C1s M17 (N380H)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMEHGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 129 - protein, 10 aa, synthetic construct
  REGION 1..10: note = Description of Artificial Sequence: Synthetic peptide
  REGION 1..10: note = Consensus HC-CDR-1 sequence by alignment of antibody 2-7; hz2-7(H1L2); and hz2-7(H1L2 G131A)
GFTFSNYFMN

SEQ ID NO: 130 - protein, 19 aa, synthetic construct
  REGION 1..19: note = Description of Artificial Sequence: Synthetic peptide
  REGION 1..19: note = Consensus HC-CDR-2 sequence by alignment of antibody 2-7; hz2-7(H1L2); and hz2-7(H1L2 G131A)
  MOD_RES 15: note = E or D
  MOD_RES 18: note = E or K
EIRLKFTNYATHYAXSVXG

SEQ ID NO: 131 - protein, 11 aa, synthetic construct
  REGION 1..11: note = Description of Artificial Sequence: Synthetic peptide
  REGION 1..11: note = Consensus HC-CDR-3 sequence by alignment of antibody 2-7; hz2-7(H1L2); and hz2-7(H1L2 G131A)
  MOD_RES 7: note = G or A
DYGSRNXYFDY

SEQ ID NO: 132 - protein, 16 aa, synthetic construct
  REGION 1..16: note = Description of Artificial Sequence: Synthetic peptide
  REGION 1..16: note = Consensus LC-CDR-1 sequence by alignment of antibody 2-7; hz2-7(H1L2); and hz2-7(H1L2 G131A)
RSSKSLLHSNGNTFLY

SEQ ID NO: 133 - protein, 7 aa, synthetic construct
  REGION 1..7: note = Description of Artificial Sequence: Synthetic peptide
  REGION 1..7: note = Consensus LC-CDR-2 sequence by alignment of antibody 2-7; hz2-7(H1L2); and hz2-7(H1L2 G131A)
RMSNLAS

SEQ ID NO: 134 - protein, 9 aa, synthetic construct
  REGION 1..9: note = Description of Artificial Sequence: Synthetic peptide
  REGION 1..9: note = Consensus LC-CDR-3 sequence by alignment of antibody 2-7; hz2-7(H1L2); and hz2-7(H1L2 G131A)
MQHLEYPYT

SEQ ID NO: 135 - protein, 10 aa, synthetic construct
  REGION 1..10: note = Description of Artificial Sequence: Synthetic peptide
  REGION 1..10: note = Consensus HC-CDR-1 sequence by alignment of antibody 2-8; hz2-8(H1L2); hz2-8(H1L2 G80A/T82A); and hz2-8(H1L2 G80A)
GFNIKEYYMH

SEQ ID NO: 136 - protein, 17 aa, synthetic construct
  REGION 1..17: note = Description of Artificial Sequence: Synthetic peptide
  REGION 1..17: note = Consensus HC-CDR-2 sequence by alignment of antibody 2-8; hz2-8(H1L2); hz2-8(H1L2 G80A/T82A); and hz2-8(H1L2 G80A)
  MOD_RES 7: note = G or A
  MOD_RES 9: note = T or A
  MOD_RES 12: note = D or A
  MOD_RES 13: note = P or Q
WIDPENXDXIYXXKFQG

SEQ ID NO: 137 - protein, 7 aa, synthetic construct
  REGION 1..7: note = Description of Artificial Sequence: Synthetic peptide
  REGION 1..7: note = Consensus HC-CDR-3 sequence by alignment of antibody 2-8; hz2-8(H1L2); hz2-8(H1L2 G80A/T82A); and hz2-8(H1L2 G80A)
SRLFFAY

SEQ ID NO: 138 - protein, 11 aa, synthetic construct
  REGION 1..11: note = Description of Artificial Sequence: Synthetic peptide
  REGION 1..11: note = Consensus LC-CDR-1 sequence by alignment of antibody 2-8; hz2-8(H1L2); hz2-8(H1L2 G80A/T82A); and hz2-8(H1L2 G80A)
  MOD_RES 1: note = K or R
  MOD_RES 10: note = V or L
XASQDVRTAXD

SEQ ID NO: 139 - protein, 7 aa, synthetic construct
  REGION 1..7: note = Description of Artificial Sequence: Synthetic peptide
  REGION 1..7: note = Consensus LC-CDR-2 sequence by alignment of antibody 2-8; hz2-8(H1L2); hz2-8(H1L2 G80A/T82A); and hz2-8(H1L2 G80A)
  MOD_RES 7: note = T or S
SASYRYX

SEQ ID NO: 140 - protein, 9 aa, synthetic construct
  REGION 1..9: note = Description of Artificial Sequence: Synthetic peptide
  REGION 1..9: note = Consensus LC-CDR-3 sequence by alignment of antibody 2-8; hz2-8(H1L2); hz2-8(H1L2 G80A/T82A); and hz2-8(H1L2 G80A)
QQQYTTPYT

SEQ ID NO: 141 - protein, 673 aa, synthetic construct
  REGION 1..673: note = Description of Artificial Sequence: Synthetic polypeptide
  REGION 1..673: note = Protein sequence of human C1s M18 (V288T)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSTWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 142 - protein, 673 aa, synthetic construct
  REGION 1..673: note = Description of Artificial Sequence: Synthetic polypeptide
  REGION 1..673: note = Protein sequence of human C1s M19 (Q303K)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVKITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 143 - protein, 673 aa, synthetic construct
  REGION 1..673: note = Description of Artificial Sequence: Synthetic polypeptide
  REGION 1..673: note = Protein sequence of human C1s M20 (A320S)-His
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGSTSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

(19) United States
(12) Patent Application Publication
(10) Pub. No.: US 2024/0158483 A1
(43) Pub. Date: May 16, 2024

(54) ANTI-C1S ANTIBODIES AND USES THEREOF

(71) Applicant: MABWELL THERAPEUTICS, INC., SAN DIEGO, CA (US)

(72) Inventors: Buxin CHEN, San Marcos, CA (US); Lei HUANG, San Diego, CA (US); Xin DU, La Jolla, CA (US)

(21) Appl. No.: 18/430,175

(22) Filed: Feb. 1, 2024

Related U.S. Application Data
(63) Continuation of application No. 17/852,326, filed on Jun. 28, 2022.
(60) Provisional application No. 63/221,131, filed on Jul. 13, 2021.
(60) Provisional application No. 63/342,544, filed on May 16, 2022.

(51) Int. Cl. C07K 16/18

(52) U.S. Cl. CPC C07K 16/18; C07K 2317/24; C07K 2317/33; C07K 2317/34; C07K 2317/565; C07K 2317/76; C07K 2317/92

(57) ABSTRACT

Antibodies and antigen-binding fragments thereof that bind C1s and inhibit C1s activity and modulate the activity of at least one component in the classical pathway (CP) of complement activation, and methods for treating complement-mediated disorders using anti-C1s antibodies and fragments, are provided.

### CROSS-REFERENCE TO RELATED APPLICATIONS

This application claims the benefit of U.S. Provisional Application No. 63/342,544 filed May 16, 2022, and U.S. Provisional Application No. 63/221,131 filed Jul. 13, 2021, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 28, 2022, is named SEQ_LIST_PIPL_1121-102US.txt and is 279,261 bytes in size.

### FIELD OF THE INVENTION

The present disclosure relates to antibodies and antigen-binding fragments that bind to C1s protein, and treating complement-mediated disorders using antibodies and antigen-binding fragments that bind C1s protein.

### BACKGROUND

The complement system is a tightly regulated network of proteins that form an important part of the innate and adaptive immune systems. With multiple activation pathways and a wide range of complement-mediated effector functions, the complement system plays a critical role in inflammation, defense against pathogens, and recovery after injury. Complement activation can have both beneficial and harmful effects. Defense responses mediated by complement activation can eliminate pathogens. Inappropriate, unwanted, excessive, or deficient complement activation has been associated with disease conditions such as certain autoimmune diseases and inflammatory syndromes, reflecting the intricate balancing act of complement between immune regulation and inflammatory tissue damage.

The complement system can be activated via three different pathways that converge at the effector stage wherein C3 convertase activates complement C3 protein and triggers a cascade of downstream effects. Each pathway has different triggers, initiator molecules, and enzyme cascades upstream of the generation of C3 convertase (1) the classical pathway (CP) can be triggered when antibody-antigen complexes bind to the C1 complex and C1 is activated; (2) the alternative pathway (AP) can be triggered by cell surface constituents that are foreign to the host; and (3) the mannose-binding lectin pathway (LP) can be triggered when circulating twin binds to mannose residues on microorganism surfaces. After the upstream cascade of each pathway generates C3 convertase, subsequent cleavage of complement proteins C3 and C5 result in: (1) generation of anaphylatoxins C3a and C5a, which attract and activate effector immune cells to the site of antibody binding/complement activation; (2) deposition of C3b opsonin that mediate phagocytosis and lymphocyte activation; and (3) triggering of the lytic pathway to form the membrane attack complex (MAC) that disrupts the cellular membrane and leads to cellular destruction.

The CP is not only critical to antibody-mediated defenses against foreign pathogens, but is also known or suspected to be involved in triggering immune responses to the engagement of autoantibody with self-antigen, i.e., autoimmune disorders. The CP is triggered by activation of the C1 complex, which normally circulates in the plasma as an inactive complex of its subcomponent proteins C1q, C1r and C1s. After recognizing and binding antibody-antigen complexes, the subcomponents of the C1 complex are sequentially activated in an enzyme cascade wherein binding C1q to the Fc regions of two antigen-bound antibodies activates C1r subunits, which in turn activates C1s by cleaving, C1s proenzyme into active C1s with two subcomponents (A and B chains) connected by an interchain disulfide bond. Activated C1s B chain is a serine protease that can, in turn, cleave serum C4 and C2 to form a C3 convertase, C4b2a. Formation of a C3 convertase in the CP then triggers downstream effector processes that are common to all three complement pathways.

C1s protein (complement component 1s) has a pivotal role in CP function, where C1s proenzyme in the inactive C1 complex must be cleaved into an active two-subunit (two-chain) C1s that can cleave and activate serum C4 in order for downstream effector cascades to proceed. For example, in human patients, deficiencies of C1s protein can lead to severe immune complex diseases because the immune complex deposits cannot be efficiently cleared without functional C1, C2 or C4, and subsequently normal levels of C3b. Some autoimmune and/or inflammatory disorders are known or suspected to involve spontaneous activation of the CP by complexes of autoantibodies bound to self-antigens, especially complement-fixing autoantibodies. Known complement-mediated disorders include paroxysmal nocturnal hemoglobinuria (PNH), atypical hemolytic uremic syndrome (atypical HUS), hereditary angioedema (HAE), age-related macular degeneration (AMD), and autoimmune hemolytic anemia such as cold agglutinin disease (CAD). A role for the complement system, in particular the CP, is suspected in certain autoimmune disorders such as immune thrombocytopenic purpura (ITP) which is an autoimmune bleeding disorder characterized by isolated thrombocytopenia with platelet count <150,000/μL, which is suspected to result from the development of autoantibody targeting of self-platelet antigens. A role for the CP is also suspected in neuromyelitis optica (NMO), autoimmune disorder with inflammatory demyelinating lesions in the central nervous system, particularly in the spinal cord and optic nerve, that are thought to result from binding of anti-aquaporin-4 (AQP4) autoantibodies to astrocytes. Therapeutic approaches to treat complement-mediated disorders have included small molecule regulators such as protease inhibitors, although some known therapies such as C1 inhibitor C1 INH are known to exert effects beyond regulation of the CP, including modulation of the lectin pathway and kinin, coagulation and fibrinolytic systems. Therapeutic approaches using antibodies against components of the CP pathway are being explored.

### SUMMARY

The invention relates to novel antibodies and antigen-binding fragments that bind human complement component 1s (C1s), and methods of making and using antibodies and antigen-binding fragments that bind C1s.

The present disclosure provides anti-C1s antibodies, anti-C1s antibody fragments that bind C1s, nucleic acids encoding anti-C1s antibodies and anti-C1s antibody fragments, and methods of making and using anti-C1s antibodies and anti-C1s antibody fragments. The term “anti-C1s antibodies ” in the present disclosure encompasses anti-C1s antibodies and antigen-binding fragments thereof that are capable of binding C1s, in particular anti-C1s antibodies and antigen-binding fragments thereof that specifically bind C1s. Anti-C1s antibodies as disclosed herein are capable of binding proenzyme human C1s (native C1s). Anti-C1s antibodies as disclosed herein are capable of binding active human C1s. Anti-C1s antibodies as disclosed herein are capable of inhibiting C1s. Anti-C1s antibodies disclosed herein are capable of binding to proenzyme C1s and inhibiting C1s. Anti-C1s antibodies disclosed herein are capable of binding to active C1s and inhibiting C1s. Anti-C1s antibodies as disclosed herein are recombinant anti-C1s antibodies or antigen-binding fragments thereof that are capable of specifically binding to human C1s and inhibiting C1s.

The present disclosure provides anti-C1s antibodies for therapeutic and diagnostic uses in vivo, ex vivo, or in vitro. Anti-C1s antibodies as disclosed herein can be used to treat disorders mediated by the function of C1s. Anti-C1s antibodies as disclosed herein can be used to treat complement-mediated disorders, in particular disorders mediated by the classical pathway (CP) of complement activation. Anti-C1s antibodies as disclosed herein are capable of binding to C1s and inhibiting C1s, having effects on the activity of the CP pathway that can be measured by effects on the activity of at least one component of the CP downstream of C1s activation, where the component may be a a molecule or a biological process associated with the CP. Anti-C1s antibodies as disclosed herein are capable of inhibiting the activity of the CP pathway. Anti-C1s antibodies as disclosed herein can be used to treat disorders mediated by the function of the CP. Anti-C1s antibodies as disclosed herein can be used to target C1s for selective inhibition of the CP in complement-mediated autoimmune and/or inflammatory disorders. Anti-C1s antibodies as disclosed herein can be used to treat complement-mediated autoimmune and/or inflammatory disorders including but not limited to immune thrombocytopenic purpura (ITP) or neuromyelitis optica (NMO).

In certain embodiments, anti-C1s antibodies disclosed herein are capable of binding to proenzyme (native) C1s and inhibiting effects of C1s activation. In certain embodiments, anti-C1s antibodies disclosed herein are capable of binding to active C1s and inhibiting effects of C1s activation. Anti-C1s antibodies disclosed herein are capable inhibiting or or more effects of C1s activation on at least one downstream component. In certain embodiments, the downstream component is at least one of cleavage of C2 and/or cleavage of C4 and/or formation of C3 covertase, and/or serum complement-induced lysis of antibody sensitized cells, and the binding of anti-C1s antibodies to active C1s has the effect of inhibiting cleavage of C2 and/or cleavage of C4 and/or formation of C3 covertase, and/or serum-induced lysis of antibody sensitized cells.

In one aspect, anti-C1s antibodies are provided that are capable of binding to C1s and modulating at least one effect of antibody-antigen complex triggered CP activation. In certain embodiments, anti-C1s antibodies disclosed herein are capable of modulating at least one effect of antibody-antigen complex triggered CP activation in a dose-dependent manner. In certain embodiments, anti-C1s antibodies disclosed herein are capable of inhibiting at least one effect of antibody-antigen complex triggered CP activation in a dose-dependent manner. In certain embodiments, anti-C1s antibodies disclosed herein are capable or inhibiting serum-induced lysis of antibody sensitized cells in a dose-dependent manner.

In one aspect, anti-C1s antibodies are provided that are capable of having the effect of modulating the activity of at least one component involved in antibody-antigen complex triggered C4 cleavage and/or deposition. In certain embodiments, anti-C1s antibodies disclosed herein are capable of inhibiting the activity of at least one component involved in IgM-triggered deposition of C4 from serum. In certain embodiments, anti-C1s antibodies disclosed herein are capable of inhibiting IgM-triggered deposition of human C4 from human serum in a dose-dependent manner. In certain embodiments, anti-C1s antibodies disclosed herein are capable of modulating the activity of at least one component involved in IgM-triggered deposition of cynomolgus monkey C4 from cynomolgus monkey serum, in a dose-dependent manner. In certain embodiments, anti-C1s antibodies disclosed herein are capable of inhibiting IgM-triggered deposition of cynomolgus monkey C4 from cynomolgus monkey serum in a dose-dependent manner.

In another aspect, anti-C1s antibodies are provided that have selective effects within the CP, upstream of the generation and activity of common complement system effectors. In certain embodiments, selective inhibition of the CP by anti-C1s antibodies disclosed herein prevents C4 cleavage and downstream effects triggered by C4 cleavage. In certain embodiments, anti-C1s antibodies disclosed herein prevent C3 convertase formation through the CP pathway.

In another aspect anti-C1s antibodies are provided that bind to C1s proenzyme and to active C1s protein. In certain embodiments, anti-C1s antibodies disclosed herein bind to C1s proenzyme and to active C1s protein with similar affinity. In certain embodiments, anti-C1s antibodies disclosed herein specifically bind to C1s proenzyme and to active C1s protein with similar high affinity, with KD values in the low nanornolar to picomolar range.

In another aspect, anti-C1s antibodies are provided that show cross-reactivity with at non-human C1s. In certain embodiments, anti-C1s antibodies disclosed herein may be capable of binding human C1s and rat C1s. In certain embodiments, anti-C1s antibodies disclosed herein may be capable of binding human C1s and cynomolgus monkey C1s. In certain embodiments, anti-C1s antibodies disclosed herein may be capable of binding human C1s, rat C1s, and cynomolgus monkey C1s. In certain embodiments, anti-C1s antibodies disclosed herein do not show detectable binding to mouse C1s.

In another aspect, anti-C1s antibodies are provided that bind to an epitope that is accessible on proenzyme C1s and on active C1s, in the internal region of C1s within a 151-amino acid region spanning from residues Y272 to R422 (M151) in the N-terminus heavy chain (NHC), with key residues including R316, K336 and a subregion of G390 to R422 of human C1s (SEQ ID NO: 99).

An anti-C1s antibody as provided herein is recombinantly expressed and is a recombinant antibody. An anti-C1s antibody as provided herein may further be one or more of a humanized antibody, a chimeric antibody, a single chain antibody, a Fab fragment, a single-chain variable fragment (scFv), an aptamer, a single-domain antibody (VHH, nanobody, camelid antibody), or any other C1s-binding fragment or variant. In particular, an anti-C1s antibody disclosed here is a humanized antibody. In certain embodiments, an anti-C1s antibody disclosed herein may comprise a framework in which amino acids have been substituted into an existing antibody framework, in particular to influence properties such as antigen-binding ability. In certain embodiments, an anti-C1s antibody disclosed herein may comprise complementarity determining regions (CDRs) from a source (parental) antibody that have been grafted (fused) into a framework from a different type (class) of antibody and/or from a different organism than the parental antibody, in particular an acceptor human framework. In certain embodiments, an anti-C1s antibody disclosed herein may comprise a framework in which amino acids have been substituted, mutated, or replaced in regions outside of the CDRs to influence properties such as antigen-binding or antibody structure, e.g., in the variable region framework surrounding the CDRs and/or in a constant region, in particular the Fc region. In certain embodiments, one or more of the CDRs have been substituted, mutated, or replaced. In certain embodiments, an anti-C1s antibody disclosed herein may be a humanized anti-C1s antibody variant.

In certain embodiments, an anti-C1s antibody disclosed herein comprises at least one polypeptide having an amino acid sequence as set forth in Table 1 (“Variable Regions of Anti-C1s Antibodies”) or the Sequence Listing, or a sequence substantially identical (e.g., a sequence at least about 85%, 90%, 92%, 95%, 97%, or 98%, identical) to an amino acid sequence as set forth in Table 1 or the Sequence Listing. Anti-C1s antibodies disclosed herein may comprise at least one polypeptide flaying an amino acid sequence selected from the following, or a sequence substantially identical (e.g., a sequence at least about 85%, 90%, 92%, 95%, 97%, or 98%, 99% identical) to at least one polypeptide having an amino acid sequence selected from the following: SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 11; SEQ ID NO: 12; SEQ ID NO: 13; SEQ ID NO: 14; SEQ ID NO: 16; SEQ ID NO: 17; SEQ ID NO: 18; SEQ ID NO: 19; SEQ ID NO: 21; SEQ ID NO: 22; SEQ ID NO: 23; SEQ ID NO: 24; SEQ ID NO: 26; SEQ ID NO: 27; SEQ ID NO: 28; SEQ ID NO: 29; SEQ ID NO: 31; SEQ ID NO: 32; SEQ ID NO: 33; SEQ ID NO: 34; SEQ ID NO: 36; SEQ ID NO: 37; SEQ ID NO: 38; SEQ ID NO: 39; SEQ ID NO: 41; SEQ ID NO: 42; SEQ ID NO: 43; SEQ ID NO: 44: SEQ ID NO: 46; SEQ ID NO: 47; SEQ ID NO: 48; SEQ ID NO: 49; SEQ ID NO: 51; SEQ ID NO: 52; SEQ ID NO: 53; SEQ ID NO: 54; SEQ ID NO: 56; SEQ ID NO: 57; SEQ ID NO: 58; SEQ ID NO: 59; SEQ NO: 61; SEQ NO: 62; SEQ NO: 63; SEQ NO: 64; SEQ NO: 66; SEQ NO: 67; SEQ ID NO: 68; SEQ NO: 69; SEQ NO: 71; SEQ NO: 73; SEQ NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; SEQ ID NO: 95; SEQ ID NO: 97; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; or SEQ ID NO: 140.

In one embodiment, an anti-C1s antibody disclosed herein comprises a heavy chain variable region (VH) polypeptide having the amino acid sequence set forth in SEQ ID NO: 1 or a sequence substantially identical to SEQ ID NO: 1, and a light chain variable region (VL) polypeptide having the amino acid sequence set forth in SEQ ID NO: 6 or a sequence substantially identical to SEQ ID NO: 6. In one embodiment, an anti-C1s antibody disclosed herein comprises a heavy chain complementarity determining region 1 (HC CDR1) having the amino acid sequence set forth in SEQ ID NO: 2, a heavy chain complementarity determining region 2 (HC CDR2) having the amino acid sequence set forth in SEQ ID NO: 3, a heavy chain complementarity determining region 3 (HC CDR3) having the amino acid sequence set forth in SEQ ID NO: 4; a light chain complementarity determining region 1 (LC CDR1) having the amino acid sequence set forth in SEQ ID NO: 7, a light chain complementarity determining region 2 (LC CDR2) having the amino acid sequence set forth in SEQ ID NO: 8, and a light chain complementarity determining region 3 (LC CDR3) having the amino acid sequence set forth in SEQ ID NO: 9; or a variant of said antibody comprising 1, 2, 3, 4, 5, or 6 amino acid substitutions in the CDR regions. In one non-limiting embodiment, an anti-C1s antibody disclosed herein is the antibody identified herein as 2-7, comprising an polypeptide having the amino acid sequence set forth SEQ ID NO: 71 and an LC polypeptide having the amino acid sequence set forth in SEQ ID NO: 73.

In one embodiment, an anti-C1s antibody disclosed herein comprises VH polypeptide having the amino acid sequence set forth in SEQ ID NO: 11 or a sequence substantially identical to SEQ ID NO: 11, and an VL polypeptide having the amino acid sequence sot forth SEQ ID NO: 16 or a sequence substantially identical to SEQ ID NO: 16. In one embodiment, an anti-C1s antibody disclosed herein comprises an HC CDR1 having the amino acid sequence set forth in SEQ ID NO: 12, an HC CDR2 having the amino acid sequence set forth in SEQ ID NO: 13, an HC CDR3 having the amino acid sequence set forth in SEQ ID NO: 14; an LC CDR1 having the amino acid sequence set forth in SEQ ID NO: 17, an LC CDR2 having the amino acid sequence set forth in SEQ ID NO: 18, and an LC CDR3 having the amino acid sequence set forth SEQ ID NO: 19, or a variant of said antibody comprising 1, 2, 3, 4, 5, or 6 amino acid substitutions in the CDR regions. In one non-limiting embodiment, an anti-C1s antibody disclosed herein is of the antibody identified herein as 2-8, comprising an HC polypeptide having the amino acid sequence set forth in SEQ ID NO: 75 and an LC polypeptide flaying the amino acid sequence set forth in SEQ ID NO: 77.

In one embodiment, an anti-C1s antibody disclosed herein comprises a VH polypeptide having the amino acid sequence set forth in SEQ ID NO: 21 or a sequence substantially identical to SEQ ID NO: 21, and a VL polypeptide having the amino acid sequence set forth in SEQ ID NO: 26 or a sequence substantially identical to SEQ ID NO: 26. In one embodiment, an anti-C1s antibody disclosed herein comprises an HC CDR1 having the amino acid sequence set forth in SEQ ID NO: 22, an HC CDR2 having the amino acid sequence set forth in SEQ ID NO: 23, an HC CDR3 having the amino acid sequence set forth in SEQ ID NO: 24; an LC CDR1 having the amino acid sequence set forth in SEQ ID NO: 27, an LC CDR2 having the amino acid sequence set forth in SEQ ID NO: 28, and an LC CDR3 having the amino acid sequence set forth in SEQ ID NO: 29, or a variant of said antibody comprising 1, 2, 3, 4, 5, or 6 amino acid substitutions in the CDR regions. In one non-limiting embodiment, an anti-C1s antibody disclosed herein is the antibody identified herein as hz2-7(H1L2), comprising an HC polypeptide having the amino acid sequence set forth in SEQ ID NO: 79 and an LC polypeptide having the amino acid sequence set forth in SEQ ID NO: 81.

In one embodiment, an anti-C is antibody disclosed herein comprises a polypeptide having the amino acid sequence set forth in SEQ ID NO: 31 or a sequence substantially identical to SEQ ID NO: 31, and a VL polypeptide having the amino acid sequence set forth in SEQ ID NO: 36 or a sequence substantially identical to SEQ ID NO: 36. In one embodiment, an anti-C1s antibody disclosed herein comprises an HC CDR1 having the amino acid sequence set forth in SEQ ID NO: 32, an HC CDR2 having the amino acid sequence set forth an SEQ ID NO: 33, an HC CDR3 having the amino acid sequence set forth in SEQ ID NO: 34; an LC CDR1 having the amino acid sequence set forth in SEQ ID NO: 37, an LC CDR2 having the amino acid sequence set forth in SEQ ID NO: 38, and an LC CDR3 having the amino acid sequence set forth in SEQ ID NO: 39, or a variant of said antibody comprising 1, 2, 3, 4, 5, or 6 amino acid substitutions in the CDR regions. In one non-limiting embodiment, an anti-C1s antibody disclosed herein is the antibody identified herein as hz2-7(H1L2G131A), comprising an HC polypeptide having the amino acid sequence set thrill in SEQ ID NO: 83 and an LC polypeptide having the amino acid sequence set forth in SEQ ID NO: 85.

In one embodiment, an anti-C1s antibody disclosed herein comprises an HC variable region polypeptide having the amino acid sequence set forth in SEQ ID NO: 41 or a sequence substantially identical to SEQ ID NO: 41, and an LC variable region polypeptide having the amino acid sequence set forth in SEQ ID NO: 46 or a sequence substantially identical to SEQ ID NO: 46. In one embodiment, an anti-C1s antibody disclosed herein comprises an HC CDR1 having the amino acid sequence set forth in SEQ ID NO: 42, an HC CDR2 having the amino acid sequence set forth in SEQ ID NO: 43, an HC CDR3 having the amino acid sequence set forth in SEQ ID NO: 44; an LC CDR1 having the amino acid sequence set forth in SEQ ID NO: 47, an LC CDR2 having the amino acid sequence set forth in SEQ ID NO: 48, and an LC CDR3 having the amino acid sequence set forth in SEQ ID NO: 49, or a variant of said antibody comprising 1, 2, 3, 4, 5, or 6 amino acid substitutions in the CDR regions. In one non-limiting embodiment, an anti C1s antibody disclosed herein is the antibody identified herein as hz2-8(H1L2), comprising an HC polypeptide having the amino acid sequence set forth in SEQ ID NO: 87 and an LC polypeptide having the amino acid sequence set forth in SEQ ID NO: 89.

In one embodiment, an anti-C1s antibody disclosed herein comprises a VH polypeptide having the amino acid sequence set forth in SEQ ID NO: 51 or a sequence substantially identical to SEQ ID NO: 51, and a VL polypeptide having the amino acid sequence set forth in SEQ ID NO: 56 or a sequence substantially identical to SEQ ID NO: 56. In one embodiment, an anti-C1s antibody disclosed herein comprises an HC CDR1 having the amino acid sequence set forth in SEQ ID NO: 52, an HC CDR2 having the amino acid sequence set forth in SEQ ID NO: 53, an HC CDR3 having the amino acid sequence set forth in SEQ ID NO: 54; an LC CDR1 having the amino acid sequence set forth in SEQ ID NO: 57, an LC CDR2 having the amino acid sequence set forth in SEQ ID NO: 58, and an LC CDR3 flaying the amino acid sequence set forth in SEQ ID NO: 59, or a variant of said antibody comprising 1, 2, 3, 4, 5, or 6 amino acid substitutions in the CDR regions. In one non-limiting embodiment, an anti-C1s antibody disclosed herein is the antibody identified herein as hz2-8 (H1L2G80A/T82A), comprising an HC polypeptide having the amino acid sequence set forth in SEQ ID NO: 91 and an LC polypeptide having the amino acid sequence set forth in SEQ ID NO: 93.

In one embodiment, an anti-C1s antibody disclosed herein comprises a VH polypeptide having the amino acid sequence set forth in SEQ ID NO: 61 or a sequence substantially identical to SEQ ID NO: 61, and a VL polypeptide having the amino acid sequence set forth in SEQ ID NO: 66 or a sequence substantially identical to SEQ ID NO: 66. In one embodiment, an anti-C1s antibody disclosed herein comprises an HC CDR1 having the amino acid sequence set forth in SEQ ID NO: 62, an HC CDR2 having the amino acid sequence set forth in SEQ ID NO: 63, an HC CDR3 having the amino acid sequence set forth in SEQ ID NO: 64; an LC CDR1 having the amino acid sequence set forth in SEQ ID NO: 67, an LC CDR2 having the amino acid sequence set forth in SEQ ID NO: 68, and an LC CDR3 having the amino acid sequence set forth in SEQ ID NO: 69, or a variant of said antibody comprising 1, 2, 3, 4, 5, or 6 amino acid substitutions in the CDR regions. In one non-limiting embodiment, an anti-C1s antibody disclosed herein is the antibody identified herein as hz2-8 (H1L2 G80A) comprising an HC polypeptide having the amino acid sequence set forth in SEQ ID NO: 95 and an LC polypeptide having the amino acid sequence set forth in SEQ ID NO: 97.

In another aspect, CDR consensus sequences of an anti-C1s antibody disclosed herein are provided. Table 1.b. discloses non-limiting exemplary embodiments of anti-C1s antibody CDR consensus sequences. In one embodiment, an antibody is provided wherein the VH comprises an HC CDR1 having an amino acid sequence set forth in SEQ ID NO: 129, an HC CDR2 having an amino acid sequence set forth in SEQ ID NO: 130, and an HC CDR3 having an amino acid sequence set forth in SEQ ID NO: 131, and the VL comprises an LC CDR1 having an amino acid sequence set forth in SEQ ID NO: 132, an LC CDR2 having an amino acid sequence set forth in SEQ ID NO: 133, and an LC CDR3 having an amino acid sequence set forth in SEQ ID NO: 134. In another embodiment, an antibody is provided wherein the VH comprises an HC CDR1 having an amino acid sequence set forth in SEQ ID NO: 135, an HC CDR2 having an amino acid sequence set forth in SEQ ID NO: 136, and an HC CDR3 having an amino acid sequence set forth in SEQ ID NO: 137, and the VL comprises an LC CDR1 having an amino acid sequence set forth in SEQ ID NO: 138, an LC CDR2 having an amino acid sequence set forth in SEQ ID NO: 139, and an LC CDR3 flaying an amino acid sequence set forth in SEQ ID NO: 140.

In another aspect, anti-C1s antibodies (including variants and fragments as disclosed herein) are provided that can be used to treat disorders mediated by CP activation, including but not limited to disorders mediated by antibody-antigen complex triggered CP activation, and in particular disorders associated with or characterized by CP activation by autoantibodies bound to self-antigen. Methods and compositions are provided for using anti-C1s antibodies disclosed herein for therapeutic uses including but not limited to treating disorders mediated by CP activation, in particular disorders characterized by CP activation by autoantibodies bound to self-antigens. Methods and compositions are provided for using anti-C1s antibodies as disclosed herein for therapeutic uses including treating ITP and/or WO. In certain embodiments, pharmaceutical compositions comprising an anti-C1s antibody disclosed herein and a suitable carrier and/or excipient are provided.

In another aspect, methods tier treating a complement-mediated disorder are provided, such methods comprising administering an effective amount of an anti-C1s antibody disclosed herein to a subject in need thereof. In accordance with this aspect, methods for treating a complement-mediated disorder comprise administering an effective amount of an anti-C1s antibody disclosed herein to have the effect of modulating or inhibiting one or more biological effects associated with antibody-antigen complex triggered CP activation. In accordance with this aspect, an effective amount of anti-C1s antibody is the amount of antibody sufficient to cause a desired level of modulation or inhibition. In certain embodiments, methods comprise administration of an effective amount of anti-C1s antibody to a subject that results in modulating one or more biological effects associated with CP activation by autoantibodies bound to self-antigen, including but not limited to reduced lysis of platelets triggered by anti-platelet antibodies and/or antibody-mediated removal of platelets.

In another aspect, methods for treating a disease or disease state in which unwanted, abnormal, inappropriate, or excessive CP activation is involved are provided, such methods comprising administering an effective amount of an anti-C1s antibody disclosed herein to a subject in need thereof.

In another aspect, methods for diagnosing or screening for a complement-mediated disorder in a subject are provided. Methods for diagnosing or screening for a complement-mediated disorder can be practiced in vivo, ex vivo, or in vitro. In certain embodiments methods comprise administering anti-C1s antibody to a subject known or suspected to have a complement-mediated autoimmune disorder and measuring one or more biological effect or symptom associated with a complement-mediated autoimmune disorder. In certain embodiments, ex vivo or in vitro methods comprise administering anti-C1s antibody to a sample taken from a subject and measuring one or more biological effect or symptom associated with a complement-mediated autoimmune disorder.

In another aspect, one or more isolated nucleic acid molecules are provided that encode at least a portion of at least one of the anti-C1s antibodies disclosed herein. In certain embodiments, isolated nucleic acid molecules that encode at least a portion of at least one of the anti-C1s antibodies disclosed herein comprise a nucleotide sequence set forth in Table 2 herein, or a sequence substantially identical (e.g., a sequence at least about 85%, 90%, 92%, 95%, 97%, or 98%, 99% identical) to a nucleotide sequence set forth in Table 2. In certain embodiments, isolated nucleic acid molecules that encode at least one of the heavy chain (HC) sequences of the anti-C1s antibodies disclosed herein may comprise a nucleotide sequence selected from at least one of: SEQ ID NO: 5 or a sequence substantially identical to SEQ ID NO: 5; SEQ ID NO: 15 or a sequence substantially identical to SEQ ID NO: 15; SEQ ID NO. 25 or a sequence substantially identical to SEQ ID NO: 25; SEQ ID NO: 35 or a sequence substantially identical to SEQ ID NO: 35; SEQ ID NO: 45 or a sequence substantially identical to SEQ ID NO: 43; SEQ ID NO: 55 or a sequence substantially identical to SEQ ID NO: 55; SEQ ID NO: 65 or a sequence substantially identical to SEQ ID NO: 65; SEQ ID NO: 72 or a sequence substantially identical to SEQ ID NO: 72; SEQ ID NO: 76 or a sequence substantially identical to SEQ ID NO: 76; SEQ ID NO: 80 or a sequence substantially identical to SEQ ID NO: 80; SEQ ID NO: 84 or a sequence substantially identical to SEQ ID NO: 84; SEQ ID NO: 88 or a sequence substantially identical to SEQ ID NO: 88; SEQ ID NO: 92 or a sequence substantially identical to SEQ ID NO 92; or SEQ ID NO: 96 or a sequence substantially identical to SEQ ID NO: 96.

In certain embodiments, isolated nucleic acid molecules that encode at least one of the light chain (LC) sequences of the anti-C1s antibodies or antigen-binding fragments thereof disclosed herein may comprise a nucleotide sequence selected from at least one of SEQ ID NO: 10 or a sequence substantially identical to SEQ ID NO: 10; SEQ ID NO: 20 or a sequence substantially identical to SEQ ID NO: 20; or SEQ ID NO: 30 or a sequence substantially identical to SEQ ID NO: 30; SEQ ID NO: 40 or a sequence substantially identical to SEQ ID NO: 40; SEQ ID NO: 50 or a sequence substantially identical to SEQ ID NO: 50; SEQ ID NO: 60 or a sequence substantially identical to SEQ ID NO: 60; SEQ ID NO: 70 or a sequence substantially identical to SEQ ID NO: 70; SEQ ID NO: 74 or a sequence substantially identical to SEQ ID NO: 74; SEQ ID NO: 78 or a sequence substantially identical to SEQ ID NO: 78; SEQ ID NO: 82 or a sequence substantially identical to SEQ ID NO: 82; SEQ ID NO: 86 or a sequence substantially identical to SEQ ID NO: 86; SEQ ID NO: 90 or a sequence substantially identical to SEQ ID NO: 90; SEQ ID NO: 94 or a sequence substantially identical to SEQ ID NO: 94; or SEQ ID NO: 98 or a sequence substantially identical to SEQ ID NO: 98.

In another aspect, a vector is provided comprising one or more nucleic acid molecules that encode at least one amino acid sequence of the anti-C1s antibodies disclosed herein. In certain embodiments, a vector is provided comprising one or more nucleic acid molecules that encode at least one of the sequences of the heavy chain (HC) or light chain (LC) of anti-C1s antibodies disclosed herein.

In another aspect, at least one host cell is provided containing a vector comprising one or more nucleic acid molecules that encode amino acid sequences of the anti-C1s antibodies disclosed herein. In certain embodiments, host cells are transiently transfected with a vector comprising one or more nucleic acid molecules that encode amino acid sequences of anti-C1s antibodies or antigen-binding fragments thereof disclosed herein, wherein the host cells are capable of supporting vector expression and recombinant production of anti-C1s antibodies or antigen-binding fragments thereof encoded by the vector.

### BRIEF DESCRIPTION OF THE FIGURES

FIG. 1 shows a schematic of the CP and proposed action of anti-C1s antibody, where Ag indicates antigen, IgG and IgM indicate antibody types that might bind Ag, C1 indicates the C1 complex wherein C1q, C1r, and C1s indicate subcomponent proteins of the C1 complex, anti-C1s indicates anti-C1s antibody binding to C1s, labels C2a, C2b, C3 C3a, C3b, C4a, C4b, and C5 indicate components of the CP cascade arranged in a schematic of steps in the cascade, and MAC indicates the membrane attack complex.

FIGS. 2A-2D show results for anti-C1s antibodies in a functional assay for the ability to inhibit IgM-induced deposition of C4 from serum. FIG. 2A shows the effects of anti-C1s antibody 2-7, anti-C1s antibody 2-8, and an antibody from non-functional clone 3-30, on IgM-induced deposition of human C4 from human serum (NHS), over the indicated range of antibody concentrations. FIG. 2B shows the effects of anti-C1s antibody 2-7 and anti-C1s antibody 2-8 on IgM-induced deposition of cynomolgus monkey C4 from cynomolgus monkey serum (CMS) over the indicated range of antibody concentrations. In FIGS. 2A-2B, symbols are used as follows: open circles represent results using an anti-C1s antibody 2-7; open triangles represent results using an anti-C1s antibody 2-8; open squares represent results using an anti-C1s antibody 3-30. FIG. 2C shows the effects of humanized anti-C1s antibody hz2-7(H1L2), humanized anti-C1s antibody hz2-80-11 L2), humanized anti-C1s antibody variant hz2-7(HIL2 G131A), humanized anti-C1s antibody variant hz2(H1L2 G80A), and humanized anti-C1s antibody variant hz2-8(H1L2 G80A/T82A) on IgM-induced deposition of human C4 from human serum (NHS), over the indicated range of antibody concentrations. FIG. 2D shows the effects of humanized anti-C1s antibody hz2-7(H1L2), humanized anti-C1s antibody hz2-8(H1L2), humanized anti-C1s antibody variant hz2-7(H1L2 G131A), humanized anti-C1s antibody variant hz2-8(H1L2 G80A), and humanized anti-C1s antibody variant hz2-8(H1L2 G80A/T82A) on IgM-induced deposition of cynomolgus monkey C4 from cynomolgus monkey serum (CMS) over the indicated range of antibody concentrations. In FIGS. 2C-2D, symbols are used as follows: open circles represent results using an anti-C1s antibody hz2-7(H1L2); black tilled circles represent results using an anti-C1s antibody hz2-7(H1L2 G131A); open triangles represent results using an anti-C1s antibody hz2-8(H1L2); black filled triangles represent results using an anti-C1s antibody hz2-8(H1L2 G80A); and triangles with diagonal lines represent results using an anti-C1s antibody hz2-8(H1L2 G80A/T82A).

FIGS. 3A-3V show results of determinations of binding affinity and cross-reactivity of anti-C1s antibodies. FIGS. 3A-3D show results using Bio-Layer interferometry technology for measuring anti-C1s antibody affinity and determining binding kinetics for active human C1s protein with the OCTET® RED96e system using AHC biosensors, by combining antibody with indicated concentrations of active human C1s protein over the times indicated, where FIG. 3A shows results for anti-C1s antibody 2-7 binding to active human C1s, FIG. 3B shows results for anti-C1s antibody 2-8 binding to active human C1s, FIG. 3C shows humanized anti-C1s antibody hz2-7(H1L2) binding to active human C1s, and FIG. 3D shows humanized anti-C1s variant hz2-8(H1L2 G80A/T82A) binding to active human C1s. FIG. 3E is a summary table showing KD, kₒₙ, k_{off} and R² values calculated for each indicated combination of antibody and target. FIGS. 3F-3G show results for measurements of anti-C1s antibody binding to active and proenzyme forms of C1s using ELISA, where FIG. 3F shows results for binding to active human C1s by the indicated concentrations of 2-7, 2-8, hz2-7(H1L2) and hz2-8(H1L2 G80A/T82A) with human IgG1 as a control, and FIG. 3G shows results for binding to human C1s proenzyme by the indicated concentrations of 2-7, 2-8, hz2-7(H1L2) and hz2-8(H1L2 G80A/T82A) with human IgG1 as a control. FIGS. 3H-3Q show results using Bio-Layer Interferometry technology for measuring anti-C1s antibody affinity and determining binding kinetics for recombinant C1s from human (HuC1s, FIGS. 3H-3K), rat (RaC1s, FIGS. 3L-3M), and cynomolgus monkey (CyC1s, FIGS. 3N-3Q) sources, where FIG. 3H shows binding by 2-7 to human C1s (HuC1s), FIG. 3I shows binding by 2-8 to HuC1s, FIG. 3J shows binding by hz2-7(H1L2) to HuC1s, FIG. 3K shows binding by hz2-8(H1L2 G80A/T82A) to HuC1s, FIG. 3L shows binding by 2-8 to RaC1s, FIG. 3M shows binding by hz2-8(H1L2 G80A/T82A) to RaC1s, FIG. 3N shows binding by 2-7 to CyC1s, FIG. 3O shows binding by 2-8 to CyC1s, FIG. 3P shows binding by hz2-7(H1L2) to CyC1s, and FIG. 3Q shows binding by hz2-8(H1L2 G80A/T82A) to Cy1s. FIG. 3R is a summary table showing KD, kₒₙ, k_{off} and R² values calculated for each indicated combination of antibody and target. FIGS. 3S-3V show results for anti-C1s antibody cross-reactivity measurements using ELISA, where the figures show binding by the indicated concentrations of 2-7, 2-8, hz2-7(H1L2), hz2-8(H1L2 G80A/T82A), and IgGs (controls) to human C1s (HuC1s, FIG. 3S), mouse C1s (MoC1s, FIG. 3T). rat C1s (RatC1s, FIG. 3U), and cynomolgus monkey C1s (CyC1s, FIG. 3V). In FIGS. 3F-3G and 3S-3V, symbols are used as follow: open circles represent results using an anti-C1s antibody 2-7; open triangles represent results using an anti-C1s antibody 2-8; open squares represent results using mouse IgG; black filled circles represent results using an anti-C1s antibody hz2-7(H1L2); black tilled triangles represent results using an anti-C1s antibody hz2-8(H1L2 G80A/T82A) and black filled squares represent results using human IgG1.

FIGS. 4A-4D show results for measurements of anti-C1s antibody binding to the full-length and different truncated forms of recombinant C1s using ELISA. FIG. 4A shows binding of 2-7, 2-8, hz2-7(H1L2), hz2-8(H1L2), and IgG controls (mouse IgG, human IgG) to full-length human C1s (HuC1s). FIG. 4B shows binding of 2-7, 2-8, hz2-7(H1L2), hz2-8(H1L2), and IgG controls (mouse IgG, human IgG1) to full-length mouse C1s (MoC1s). FIG. 4C shows binding of 2-7, 2-8, hz2-7(H1L2), hz2-8(H1L2), and IgG controls (mouse IgG, human IgG1) to truncated human C1s (HuC1s(NHC)). FIG. 4D shows binding of 2-7, 2-8, hz2-7(H1L2), hz2-8(H1L2), and IgG controls (mouse IgG, human IgG1) to truncated human C1s M151 (HuC1s(M151)). In FIGS. 4A-4D, symbols are used as follows: open circles represent results using an anti-C1s antibody 2-7; open triangles represent results using an anti-C1s antibody 2-8; open squares represent results using mouse IgG; black filled circles represent results using an anti-C1s antibody hz2-7(H1L2); black filled triangles represent results using an anti-C1s antibody hz2-8(H1L2); and black filled squares represent results using human IgG1,

FIGS. 5A-5B show binding to and schematic comparison of full-length and truncated forms (including deletion mutants) of C1s. FIG. 5A shows an image of binding of a representative anti-C1s antibody 2-8 to the full-length and different truncated forms of C1s measured using western blotting. 200 ng full-length human C1s (HuC1s) in lane 1, 200 ng full length mouse C1s (MoC1s) in lane 2, 200 ng truncated human C1s M151 (HuC1s(M151)) in lane 3, and 200 ng truncated human C1s NHC (HuC1s(NHC)) in lane 4 were resolved on SDS-PAGE under non-reducing (left blot) and reducing fright blot) conditions, followed by a transfer to nitrocellulose membrane (blots). Blocked blots were incubated with antibody 2-8 and developed with HRP-labelled secondary antibodies. In the blot of the gel running without reducing agent (left), lane 1 shows antibody 2-8 binding to full-length human C1s (HuC1s, upper band) and auto-cleaved C1s heavy chain (lower band), lane 2 shows no binding of antibody 2-8 to full length mouse C1s (MoC1s) (no detectable antibody binding), lane 3 shows antibody 2-8 binding to multiple isoforms of truncated human C1s M131 (HuC1s(M151)), and lane 4 shows antibody 2-8 binding to truncated human C1s NHC (HuC1s(NHC)). On the blot of the gel running with reducing agent (right), lane 1 shows a low level of antibody 2-8 binding to an auto-cleaved C1s heavy chain from the full-length human C1s (HuC1s), lane 2 shows no binding of antibody 2-8 to full length mouse C1s (MoC1s) (no detectable binding), lane 3 shows antibody 2-8 binding to truncated human C1s M151 (HuC1s(M151)), and lane 4 shows antibody 2-8 binding to truncated human C1s NHC (HuC1s(NHC)). FIG. 5B shows a schematic comparison and alignment of full-length human C1s, HuC1s (e.g., as exemplified in SEQ ID NO: 99), human C1s deletion mutant 1 (M151) designated HuC1s(M151) or M151 (e.g., as exemplified in SEQ ID NO: 107), human deletion mutant 2 (NHC) designated HuC1s(NHC) or NHC (e.g., as exemplified in SEQ ID NO: 109) and human C1s deletion mutant 3 (NHCΔ33) designated human C1s (NHCΔ33) or NHCΔ33 (e.g., as exemplified in SEQ ID NO:111). FIG. 5B also shows the location of point mutations introduced into HuC1s, M151, NHC, and NHCΔ33 in Example 6.

FIGS. 6A-6U shows results for binding measurements of hz2-7(H1L2), hz2-8(H1L2 G80A), hz2-8(H1L2 G80A/T82A) and human IgG1 (control) to NHCΔ33 and point mutants M1-M20 of human C1s using ELISA as follows: FIG. 6A shows binding to NHCΔ33; FIG. 6B shows binding to M1 (M277I); FIG. 6C shows binding to M2 (M277I & K331Q); FIG. 6D shows binding to M3 (M277I, P278S & N329D); FIG. 6E shows binding to M4 (P280A & E282K); FIG. 6F shows binding to M5 (K336G); FIG. 6G shows binding to M6 (P280A, E282K K336G); FIG. 6H shows binding to M7 (D343Y & E351A); FIG. 6I shows binding to M8 (E351A); FIG. 6J shows binding to M9 (K336G, D343Y & E351 A); FIG. 6K shows binding to M10 (D283I & P285A); FIG. 6L shows binding to M11 (S349P); FIG. 6M shows binding to M12 (G381E & G382E); FIG. 6N shows binding to M13 (A292D); FIG. 6O shows binding to M14 (R316H); FIG. 6P shows binding to M15 (S360N); FIG. 6Q shows binding to M16 (R368H); FIG. 6R shows binding to M17 (N380H), FIG. 6S shows binding to M18 (V288T); FIG. 6T shows binding to M19 (Q303K); and FIG. 6U shows binding to M20 (A320S). In FIGS. 6A to 6U, symbols are used as follows: open circles represent results using an anti-C1s antibody hz2-7(H1L2); black filled triangles represent results using an anti-C1s antibody hz2-8(H1L2 G80A); triangles with diagonal lines represent results using an anti-C1s antibody hz2-8(H1L2 G80A/T82A), and open squares represent results using human IgG1 (HuIgG1).

FIGS. 7A-7B show results for anti-C1s antibodies for the ability to inhibit complement-mediated lysis of antibody sensitized sheep erythrocytes (EA cells). The effects of anti-C1s antibodies hz2-7(H1L2), hz2-8(H1L2), hz2-8(H1L2 G80A) and hz2-8(H1L2 G80A/T82A), as well as an isotype control human IgG1, on normal human serum (NHS, FIG. 7A) or cynomolgus monkey serum (CMS, FIG. 7B) induced lysis of EA cells are shown. Symbols are used as follows: open circles represent results using an anti-C1s antibody hz2-7(H1L2); open triangles represent results using an anti-C1s antibody hz2-8(H1L2); black filled triangles represent results using an anti-C1s antibody hz2-8(H1L2 G80A); triangles with diagonal lines represent results using an anti-C1s antibody hz2-8(H1L2 G80A/T82A), and open squares represent results using human IgG1.

### DETAILED DESCRIPTION

The invention relates to novel antibodies and antigen-binding fragments thereof that bind C1s, and methods of making and using the same.

### Terminology/Definitions

Scientific and technical terms used in connection with the present invention shall have the meanings that are commonly understood by those of ordinary skill in the art, unless otherwise defined. Use of singular terms (“a” or “an” or “the” or other use of a term in the singular) include plural reference, and plural terms shall include the singular, unless the context clearly dictates otherwise. Thus, for example, reference to “an antibody” includes “one or more” antibodies or a “plurality” of such antibodies. All publications mentioned herein are hereby incorporated by reference in their entirety.

Generally, nomenclature and techniques of molecular biology, microbiology, cell and tissue culture, protein and nucleotide chemistry, and recombinant DNA techniques available to one of skill of the art can be employed for the antibodies, antigen-binding fragments, compositions, and methods disclosed herein. Techniques and procedures described herein are generally performed according to conventional methods well known in the art and as described in various general and more specific references, inter alia, Sambrook et al. (1989) MOLECULAR CLONING: A LABORATORY MANUAL (2nd ed., Cold Spring Harbor Laboratory Press, Cold Spring Harbor, N.Y.) and. Ausubel et al. (1994) CURRENT PROTOCOLS IN MOLECULAR BIOLOGY, Volumes I-III (John Wiley & Sons, N.Y.). Enzymatic reactions and purification techniques are performed according to manufacturer's specifications or as commonly accomplished in the art or as described herein, unless otherwise specified herein. Techniques and methods for pharmaceutical preparation and formulation, and treatment of subjects, are described herein using conventional nomenclature.

“Antibody” refers in the broadest sense to a polypeptide or combination of polypeptides that recognizes and binds to an antigen through one or more immunoglobulin variable regions, where the immunoglobulin variable regions may be naturally occurring or non-naturally occurring, e.g., as a result of engineering, chimerization, humanization, optimization, CDR-grafting, or affinity maturation.

An “antibody” as disclosed herein can be a whole (intact, full length) antibody, a single chain antibody, or an antigen binding fragment with one or two chains, and can be naturally occurring and non-naturally occurring. An antibody comprises at least sufficient complementarity determining regions (CDR), interspersed with framework regions (FR), for the antibody to recognize and bind to an antigen. An anti-C1s antibody disclosed herein may be, but is not limited to, at least one of a monoclonal antibody, a polyclonal antibody, a humanized antibody, a chimeric antibody, a single chain antibody, a Fab fragment, a single-chain variable fragment (scFv), an aptamer, a single-domain antibody (VHH, nanobody, camelid antibody), a recombinant antibody, a modified antibody having peptide/other moieties attached to antibody and/or additional amino acids added the N- or C-terminus, or other C1s-binding fragment or variant. Whole antibody, full length antibody, intact antibody, naturally occurring antibody, or equivalent terms are understood to refer to a polypeptide, in particular a glycoprotein, comprising in its canonical form with at least two heavy chains (HCs) and two light chains (LCs) interconnected by disulfide bonds. Each HC is comprised of a heavy chain variable region (VH) and an fie constant region (CH), and each light chain is comprised of a light chain variable region (VL) and an LC constant region (CL). The HC and LC variable regions, VH and VL, include a binding domain that interacts with an antigen. The VH and VL regions can be further subdivided into CDR regions characterized by hypervariability, interspersed with FR regions that are typically more conserved. Each VH and VL is typically composed of three CDRs and four FRs arranged from amino-terminus to carboxy-terminus in the following order: FR1, CDR1, FR2, CDR2, FR3, CDR3, FR4. It is understood that the assignment of amino acids to each domain is in accordance with methods known in the art, in particular in accordance with the definitions found in, inter alio, SEQUENCES OF PROTEINS OF IMMUNOLOGICAL INTEREST, Kabat, et al.; National Institutes of Health, Bethesda, Md.; 5^{th} ed., NIH Publ. No. 91-3242 (1991); Chothia, et al., (1987) J Mol. Biol. 196:901-917 or Chothia, et al., (1989) Nature 342:878-883. The constant regions of the antibodies may mediate the binding of the immunoglobulin to host tissues or factors, including various cells of the immune system and the classical complement system. Typically, an antibody comprises at least heavy chain (HC) CDR1, CDR2, and CDR3 and light chain (LC) CDR1, CDR2, and CDR3 sequences, where any one of these sequences may be naturally or non-naturally occurring. An antibody may comprise fewer CDR sequences, as long as the antibody can recognize and bind an antigen. Alternately, a whole antibody may be a single chain antibody such as a heavy-chain only isotype where antigen binding is mediated by one variable domain.

An anti-C1s antibody disclosed herein may be a variant comprising at least one altered CDR or framework sequence, wherein CDR and/or framework sequences may by optimized by mutating a nucleic acid molecule encoding such framework sequence. Variants may be constructed with HC and LC portions derived independently from different sources. Techniques for generating variants include but are not limited to conservative amino acid substitution, computer modeling, screening candidate polypeptides alone or in combinations, and codon optimization, and it is understood that a skilled person is capable of generating antibody variants as may be needed. An anti-C1s antibody disclosed herein may be a fragment. Antigen binding functions of an antibody can be performed by fragments such as: a Fab fragment; a monovalent fragment consisting of the VH, VH, CL and CH1 domains; a F(ab)₂ fragment; a bivalent fragment comprising two Fab fragments linked by a disulfide bridge at the hinge region; an Fd fragment consisting of the VH and CH1 domains; a single-chain variable fragment (scFv) consisting of the VL and VH domains of a single arm of an antibody; a single domain antibody (dAb) fragment which consists of a VH domain; and an isolated CDR (VHH, nanobody), or an aptamer. Antigen binding portions can be incorporated into single domain antibodies, maxibodies, minibodies, nanobodies, intrabodies, diabodies, triabodies, tetrabodies, v-NAR and bis-scFv (see, e.g., Hollinger and Hudson, 2005, Nature Biotechnology, 23(9): 1126-1136). Antigen binding portions of antibodies can be grafted into scaffolds based on polypeptides to form monobodies (see, e.g., U.S. Pat. No. 6,703,199, which describes fibronectin polypeptide monobodies).

The term antibody encompasses various broad classes of polypeptides that can be distinguished biochemically. The “class” of an antibody may refer to the type of constant domain or constant region possessed by its heavy chain. Those skilled in the art understand that there are five major classes of antibodies, viz., IgA, IgD, IgE, IgG, and IgM, and several of these may be further divided into subclasses (isotypes), e.g., IgG1, IgG2, IgG3, IgG4, IgA1, and IgA2, each of which is well characterized and known to confer functional specialization. Modified versions of each of these classes and isotypes are readily discernible and within the scope of the instant disclosure. All immunoglobulin classes are within the scope of the present disclosure.

The term “chimeric” antibody refers to an antibody in which a portion of the heavy chain (HC) and/or light chain (LC) involved in forming the immunoreactive site is derived from a particular source or species, while the remainder of the HC and/or LC is derived from a different source or species. In certain embodiments the target binding region or site will be from a non-human source (e.g., mouse or non-human primate) and the constant region is from human source.

As used herein, the phrase “humanized antibody” refers to am antibody (immunoglobulin) or antibody variant comprising portions of antibodies (immunoglobulins) comprising amino acid sequences of different origins, wherein at least one portion comprises amino acid sequences of human origin. A humanized antibody comprises a portion derived from a non-human antibody, typically from a mouse antibody that binds a target of interest, where the portion from non-human antibody may be an entire variable region from the “parental” non-human antibody or part of a variable region such as one or more CDRs from the “parental” non-human antibody. A humanized antibody may comprise one or more variable regions from a non-human antibody connected to an immunoglobulin framework of human origin, in particular to a human variable region framework. A humanized antibody may comprise CDRs from the parental, non-human antibody grafted (fused) in a framework comprising portions of variable regions derived from a human immunoglobulin framework, in particular an acceptor human framework or a human consensus framework. Techniques and principles for designing, making, and testing humanized antibodies are known (Jones P T, Dear P H, Foote J, Neuberger M S, Winter G. Replacing the complementarity-determining regions in a human antibody with those from a mouse. Nature. 1986, 321(60(9):522.5; Almagro J C, Fransson J. Humanization of antibodies. Front Biosci. 2008, 13:1619-33). It is understood that changes can be made to one or more CDR sequences and/or to an acceptor framework at multiple locations in order to develop a humanized antibody having improved features according to the desired use, e.g., high affinity for target, specificity for selected epitopes, avoidance of unwanted events such as isomerization or deamindation, low clearance, low toxicity, etc. An anti-C1s antibody disclosed herein may be a humanized variant.

“Binding” refers to a direct association between two molecules, due to, for example, covalent, electrostatic, hydrophobic, and ionic and/or hydrogen-bond interactions, including interactions such as salt bridges and water bridges. “Affinity” refers to the strength of the sum total of noncovalent interactions between a single binding site of a molecule (e.g., an antibody) and its binding partner (e.g., an antigen). Unless indicated otherwise, binding affinity as used herein refers to intrinsic binding affinity which reflects a 1:1 interaction between members of a binding pair (e.g., antibody and antigen). Affinity can be measured by common methods known in the art, including those described herein. The calculated concentration at which approximately 50% of maximal binding (the calculated EC₅₀) can be used as an estimate of affinity. The affinity of a molecule X for its partner Y can generally be represented by the dissociation constant (Kd or KD, representing k_{off}/kₒₙ measured for the interaction). “Specific binding” or “specifically binds” or “binds specifically” or similar terms refer to high-affinity binding, in particular binding with a measured affinity (Kd, KD) in at least the nanomolar range. An anti-C1s antibody of the present disclosure binds specifically to C1s protein.

“Substantially identical” as used to refer to a sequence substantially identical to an identified amino acid sequence or nucleotide sequence, is understood to be a sequence that is at least about 85%, 90%, 97%, 95%, 97%, or 98%, 99% identical to the amino acid or nucleotide sequence, determined as the percentage of residues in a candidate amino acid or nucleic acid sequence that is identical with the residues in the identified reference sequence after aligning the two sequences and introducing gaps, if necessary, to achieve the maximum percent identity. Methods and computer programs for the alignment are well known in the art, for example and publicly available computer software/programs such as Clustal Omega (from the European Molecular Biology Laboratory European Bioinformatics Institute (EMBL-EBI), available at https://www.ebi.ac.uk/Tools/msa/clustalo/); when using such software/programs, the default parameters, e.g. tear gap penalty and extension penalty, are preferably used

A “subject” is a mammal, where mammals include but are not limited to primates (e.g., humans and non-human primates such as monkeys), domesticated animals (e.g., cows, sheep, cats, dogs, pigs, llamas, and horses), rabbits, and rodents (e.g., mice and rats). In certain embodiments, the subject is a human. The phrases “to a subject in need thereof” or “to a patient in need thereof” or “to a patient in need of treatment” or “a subject in need of treatment” may include subjects that would benefit from administration of the anti-C1s antibodies disclosed herein, for treatment of a complement-mediated disorder. It is understood that administration of anti-C1s antibodies encompasses administration to “a subject in need thereof” can be interpreted as referring to a subject known or suspected to have a complement-mediated disorder, such as ITP or NMO, based on indicators such as symptoms, family history, or genotype. It is further understood that anti-C1s antibodies can be administered to a subject that is not known or suspected to have a complement-mediated disorder, for purposes that may include but are not limited to, for preventative or prophylactic purposes, for screening, for diagnostics, for research purposes, or to achieve results distinct from treating a disorder.

An “effective amount” of an anti-C1s antibody, e.g., in a pharmaceutical formulation, refers to an amount effective, at dosages and for periods of time necessary, to achieve the desired therapeutic or prophylactic result. It is understood that “effective amount” is intended to refer to the amount of an anti-C1s antibody or a pharmaceutical composition comprising an anti-C1s antibody that will elicit the biological response of, or desired therapeutic effect on, a cell, a tissue, a system, a non-human animal subject, a non-human mammal subject, or a human subject that is being measured. The terms “therapeutically effective amount”, “pharmacologically effective amount”, and “physiologically effective amount” are used interchangeably to refer to the amount of an anti-C1s antibody that is needed to provide threshold level of active antibody in the bloodstream or in the target tissue. The precise amount will depend upon numerous factors, e.g., the particular anti-C1s antibody (active agent), the components and physical characteristics of the composition, intended population of subjects/patients to be treated, considerations such as the disease state, age, sex, and weight of a subject, and the like, and can readily be determined by one skilled in the art, based upon the information provided herein or otherwise available in the relevant literature. The terms, “improve”, “increase” or “reduce”, as used in this context, indicate values or parameters relative to a baseline measurement, such as a measurement in the same subject prior to initiation of the treatment described herein, or a measurement in a control individual (or multiple control individuals) in the absence of the treatment described herein, or a value available in an information source such as a textbook, manual, or database.

The term “pharmaceutical composition” or “pharmaceutical formulation” refers to a preparation which is in such form as to permit the biological activity of an active ingredient contained therein to be effective, in particular an anti-C1s antibody as disclosed herein. It is understood that a pharmaceutical composition may contain more than one active ingredient, e.g., more than one anti-C1s antibody, or a combination of an anti-C1s antibody with another active ingredient that acts on a different target, where such combinations can be but are not limited to, a combination of an anti-C1s antibody with another active ingredient having a desired effect on other complement pathways or other processes involved in inflammation, a combination of an anti-C1s antibody with gene therapy agents, or a combination of an anti-C1s antibody with proteins (e.g., Fc-fusion proteins) against other targets. A “pharmaceutically acceptable carrier” refers to an ingredient in a pharmaceutical formulation, other than an active ingredient, which is nontoxic to a subject. It is understood that a pharmaceutically acceptable carrier can be, but is not limited to, a buffer, excipient, stabilizer, an adjuvant, or preservative.

The term “treat” or “treating” or similar terms as used herein, can refer to an outcome that is deemed beneficial for a particular subject in a defined set of circumstances. Treating a complement-mediated disorder may refer non-exclusively to any of reducing, ameliorating, slowing, interrupting, arresting, alleviating, stopping, or reversing the progression or severity of an existing symptom, disorder, condition, or disease, and may further encompass prevention or delay of the onset of one or more symptoms of a complement-mediated disorder, and/or lessening of the severity or frequency of one or more symptoms of a complement-mediated disorder. The terms “treating” or “method of treating” or equivalents can encompass one or more uses of anti-C1s antibodies disclosed herein, including but not limited to therapeutic, prophylactic, preventive, diagnostic, imaging, and screening uses.

The term “vector,” as used herein, refers to a nucleic acid molecule capable of propagating a nucleic acid to which the vector sequence is linked, in a host cell in which the vector is introduced. Vectors capable of directing the expression of nucleic acids to which they are operatively linked are referred to herein as “expression vectors.”

### Anti-C1s Antibodies

Antibodies and antigen-binding fragments are provided that are capable of binding C1s and modulating the activity of C1s, thereby having the effect of modulating the activity of at least one component involved in at least one complement-mediated disorder, in particular at least one disorder associated with CP activation by autoantibodies bound to self-antigens. Antibodies and antigen-binding fragments are provided that are capable of binding C1s and modulating the activity of C1s, thereby having the effect of modulating the activity of at least one component of the CP involved in ITP and/or NMO. Anti-C1s antibodies that are capable of binding C1s and modulating the activity of C1s can be used in methods and compositions for treating complement-mediated disorders associated with CP activation by autoantibodies bound to self-antigens, in particular ITP.

Once antibodies or fragments specific for C1s are provided, the desired biological activity of modulating the activity of C1s or having the effect of modulating at least one component involved in complement-mediated disorders can be tested by several methods known to the skilled person.

It is understood that “modulate” or “modulating” or similar terms as used herein can refer to one or more effects that can result when an anti-C1s antibody disclosed herein binds its target molecule C1s. “Modulating” and its equivalents can refer to different modes of action and effects depending on the component under consideration, i.e., modulating can refer to neutralizing, reversing, inhibiting, blocking, reducing, antagonizing, agonizing, amplifying, enhancing, or otherwise changing with the activity of at least one component involved in the complement system, in particular the CP. It is understood that “inhibit C1s” or “inhibiting C1s” or “inhibition of C1s” or “C1s inhibition” or similar terms as used herein, refer to inhibiting at least one activity or function of C1s involved the complement cascade, such that inhibiting C1s has downstream effects such as inhibiting the activation of enzymes involved in the complement cascade, the association or disassociation of molecules involved in the complement cascade, the production of downstream effectors that require C1s activation, or the degree of serum complement-induced lysis of antibody sensitized cells. It is understood “C1s inhibition” or similar terms as used herein do not require demonstration of the specific activity or function(s) C1s that are inhibited when the present C1s antibodies bind to the C1s target molecule, as measurements showing inhibition of processes or effectors that are known to be downstream of C1s activation can be used as surrogate measurements or indicators of C1s activity or function, and thus, as indicators of inhibition of C1s activity or function. Accordingly, C1s antibodies capable of binding to and inhibiting C1s are antibodies capable of, inter alia, inhibiting the activity of the classical pathway (CP) of complement activation, inhibiting downstream effects of C1s activation, inhibiting IgM-induced deposition of C4 from serum, inhibiting antibody-antigen complex mediated C4 cleavage, inhibiting antibody-antigen complex mediated deposition of C4b, inhibiting formation of C3 convertase, inhibiting downstream effects of CP activation, usw.

It is understood that the term “component” can refer not only to target molecule C1s, but also to a downstream molecule, process, or pathway involved in complement-mediated effects downstream of C1s activation, in particular CP-mediated effects downstream of C1s activation. It is understood that by targeting C1s with anti-C1s antibodies, the CP can be selectively targeted, such that CP-mediated effects can be specifically or selectively inhibited. This effect of selective targeting of the CP is also understood to leave the alternative and lectin pathways intact to combat infection. It is understood that anti-C1s antibodies as disclosed herein can be used to therapeutically target antibody-antigen complex triggered CP activation and modulate the activity of the CP pursuant to antibody-antigen complex triggered CP activation. Anti-C1s antibodies as disclosed herein can be used to therapeutically target at least one component involved in complement-mediated disorders, in particular to specifically target at least one component involved in CP-mediated disorders, in particular at least one component involved in CP-mediated autoimmune disorders, in particular at least one component involved in ITP and/or NMO. Without wishing to be bound by a particular mechanism of action, targeting C1s using anti-C1s antibodies as disclosed herein is understood to specifically inhibit CP mediated cell lysis, which underlies the autoimmune pathology that results in effects such as destruction of self platelets, red cells, or astrocytes. In certain embodiments, anti-C1s antibodies as disclosed herein can be used to inhibit the CP pathway in a dose-dependent manner. In certain embodiments, anti-C1s antibodies as disclosed herein can be used to inhibit the effects of antibody-antigen complex triggered CP activation in a dose-dependent manner.

In certain embodiments, anti-C1s antibodies as disclosed herein can be used to therapeutically target antibody-antigen complex triggered CP activation and modulate the activity of the CP downstream of antibody-antigen complex triggered CP activation. In particular, anti-C1s antibodies as disclosed herein can be used to therapeutically target antibody-antigen complex triggered CP activation and inhibit the activity of the CP downstream of antibody-antigen complex triggered CP activation.

Using anti-C1s antibodies as disclosed herein to therapeutically target at least one component involved in CP-mediated disorders is understood to allow precise modulation of the activity of the CP and downstream effects of CP activity. In particular, anti-C1s antibodies as disclosed herein to therapeutically target at least one component involved in CP-mediated disorders is understood to allow precise inhibition of the activity of the CP and downstream effects of CP activity.

Anti-C1s antibodies disclosed herein allow the development of treatments that can be tailored to each subject (e.g., dosage, frequency of administration), where they can be continued and discontinued with ease, and combined with other therapies. In certain strategic embodiments, anti-C1s antibodies disclosed herein can be combined with other therapies that may address multiple therapeutic targets and/or address deficits or undesirable effects of one of the therapies in the combination therapy.

### Exemplary Embodiments of Anti-C1s Antibodies and Uses Thereof

Non-limiting exemplary embodiments of anti-C1s antibodies of the invention are presently disclosed, in particular in the Examples, Tables, and Figures.

### Antibodies Capable of Specifically Binding C1s and Inhibiting C1s Function

A functional cascade can be used to identify and characterize anti-C1antibodies of the present invention, where a first step in the cascade involves screening C1s-challenged clones for antibodies capable of binding to C1s, followed by sequencing cognate variable regions, recombinantly expressing chimeric antibodies with VH and VL from mouse clones linked with human constant regions, purifying recombinant antibodies, and screening the antibodies using a functional assay.

As demonstrated by exemplary embodiments disclosed in Example 2, recombinant chimeric antibodies were obtained that were capable of modulating C1s functions, as measured by an assay for IgM-induced C4 deposition from serum. Exemplary recombinant chimeric anti-C1s antibodies were obtained that were capable of inhibiting C1s functions as measured by the ability to inhibit IgM-induced human C4 deposition from human serum, and the ability to inhibit IgM-induced cynomolgus monkey C4 deposition from cynomolgus monkey serum. Exemplary recombinant chimeric anti-C1s antibodies were obtained that were capable of inhibiting, C1s functions in a dose-dependent manner.

### Humanized Variants

Humanized antibodies comprising CDRs derived from a non-human source grafted into a human-derived antibody framework are expected to be non-immunogenic when administered to a human subject. As demonstrated by exemplary embodiments disclosed in Example 2, humanized anti-C1s antibody variants were successfully generated, tested, optimized, and selected. Humanization of the 2-7 and 2-8 antibodies yielded humanized anti-C1s antibody, variants, with the antibodies identified as hz2-7(H1L2) and hz2-8(H1L2) having the highest in vitro activities. After initial design and testing, variants that showed desired antigen binding affinity and cross-reactivity were selected for further evaluation and development, including but not limited to modification of some parental CDR sequences to avoid potential unwanted events such as aspartate isomerization and asparagine deamidation, and selected modifications of framework sequences.

### Anti-C1s Antibodies Having High Affinity for a Biologically Relevant Target

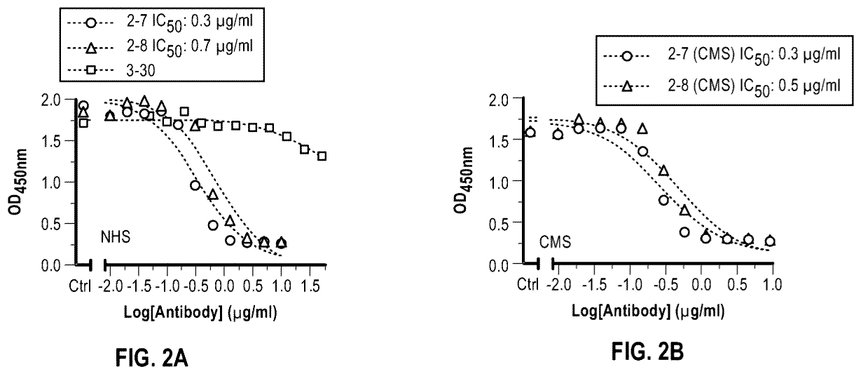

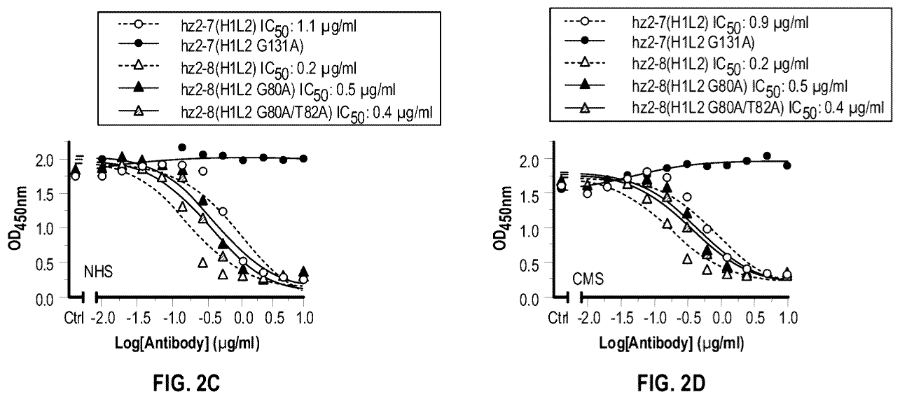

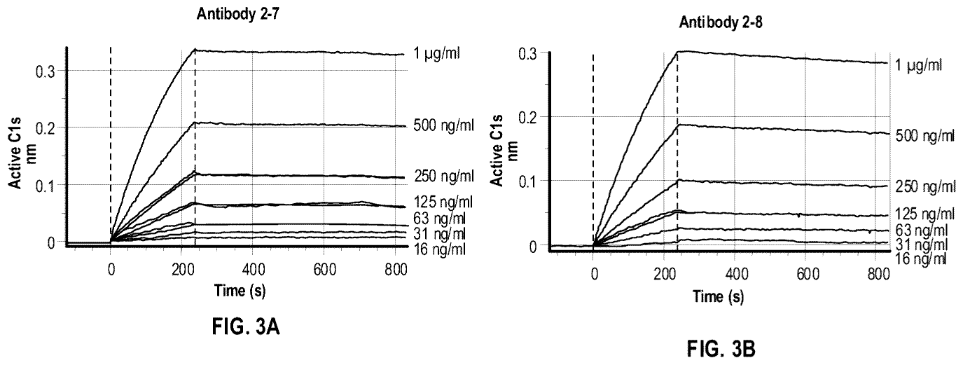

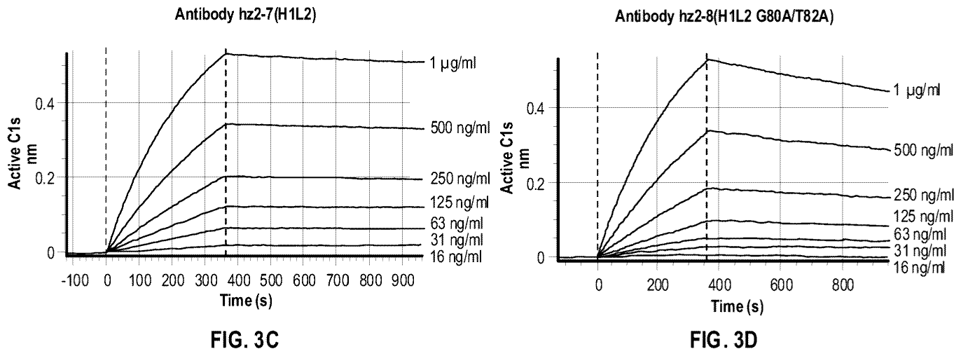

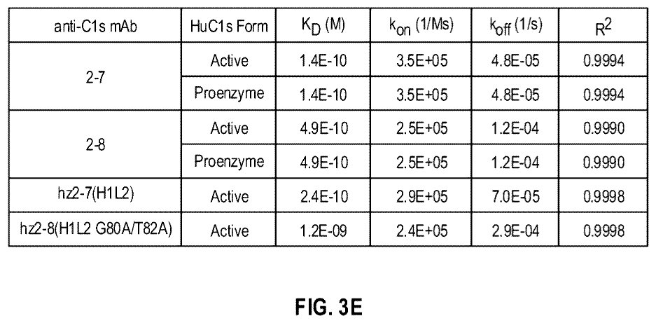

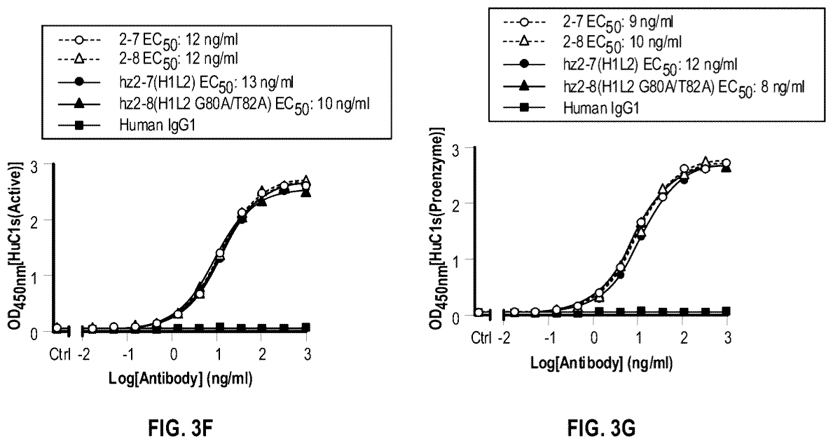

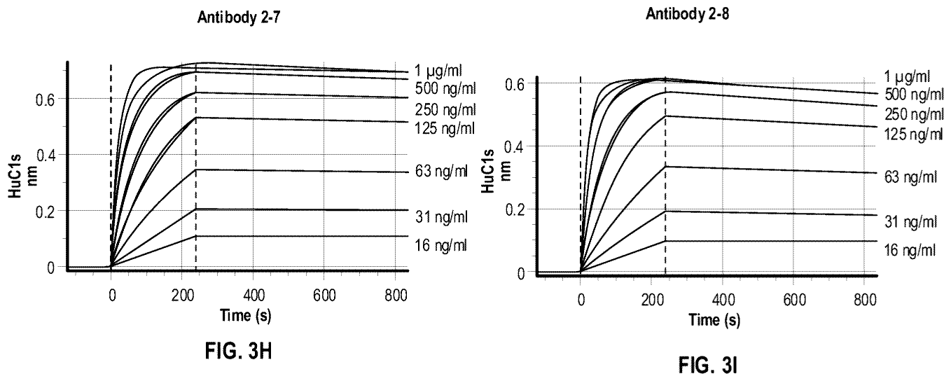

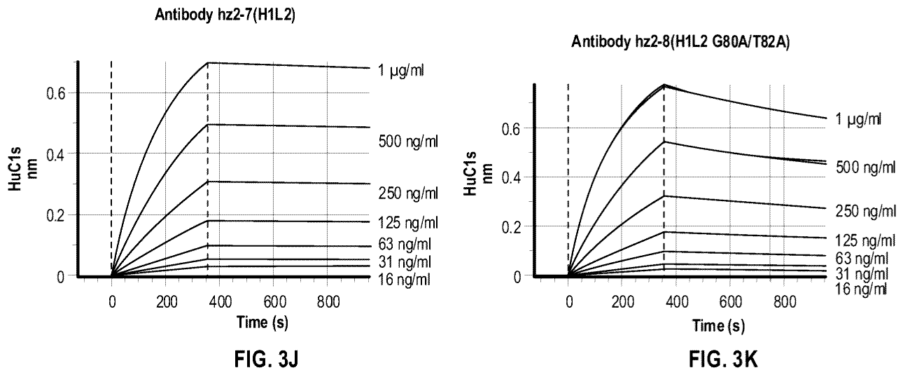

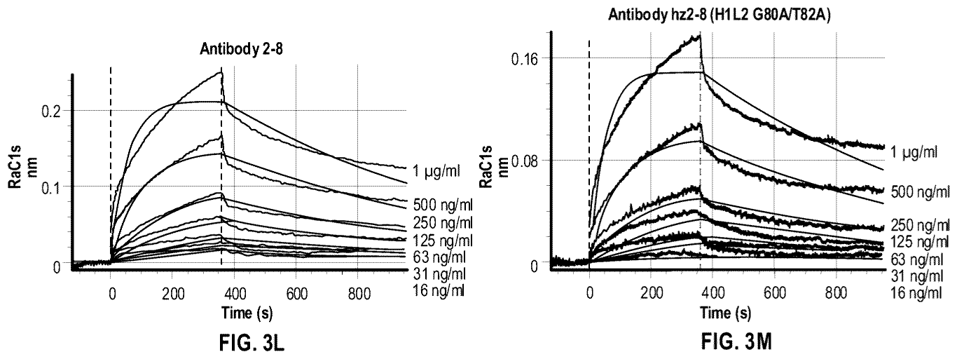

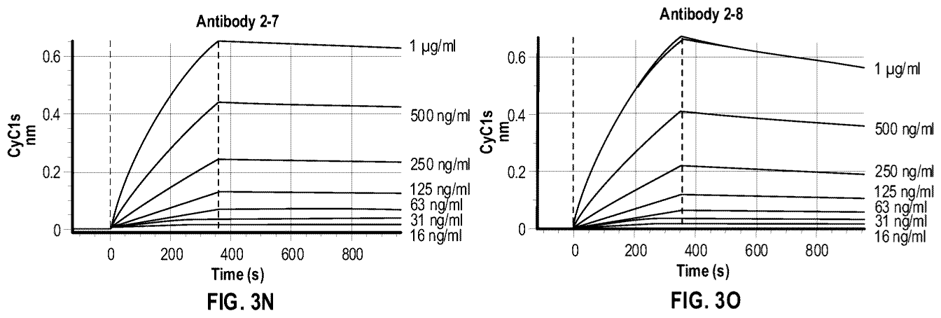

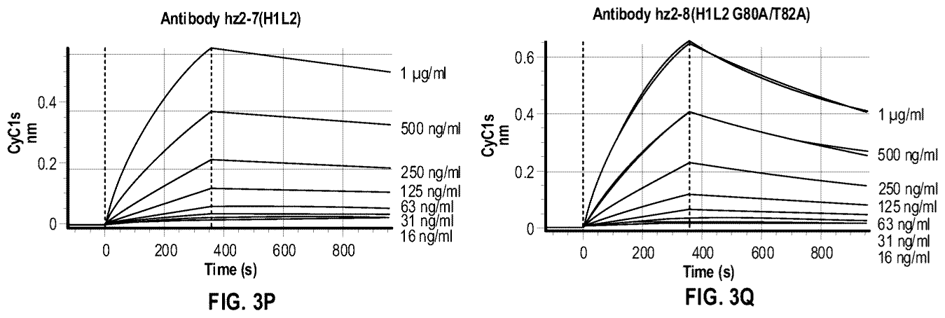

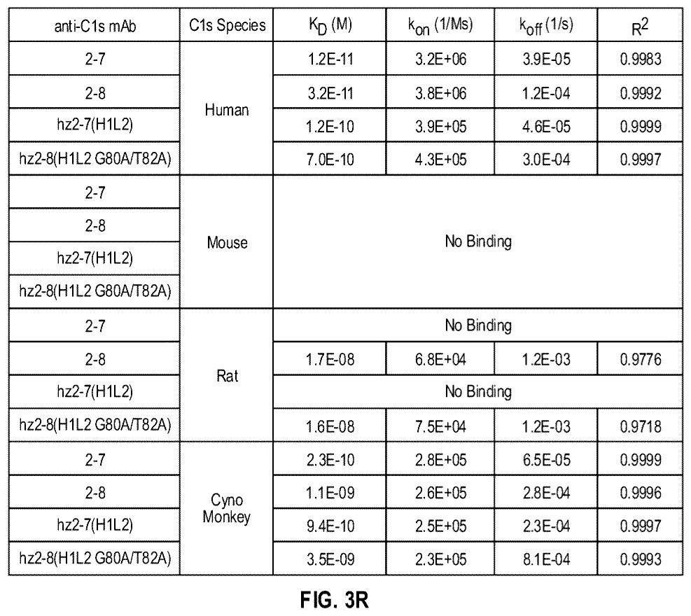

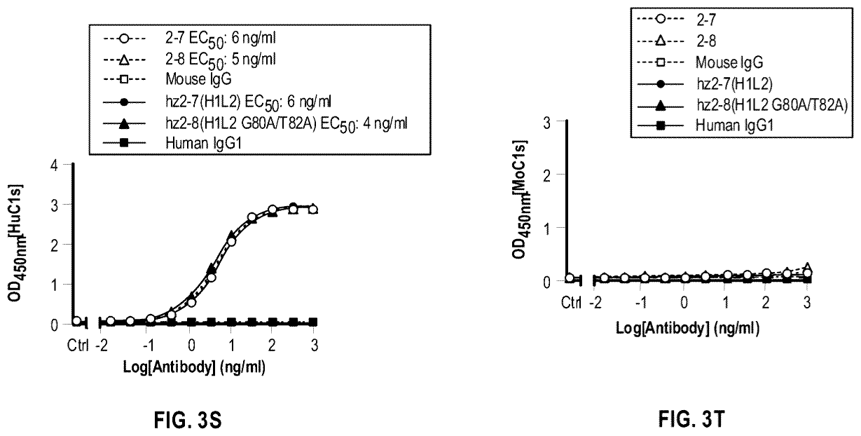

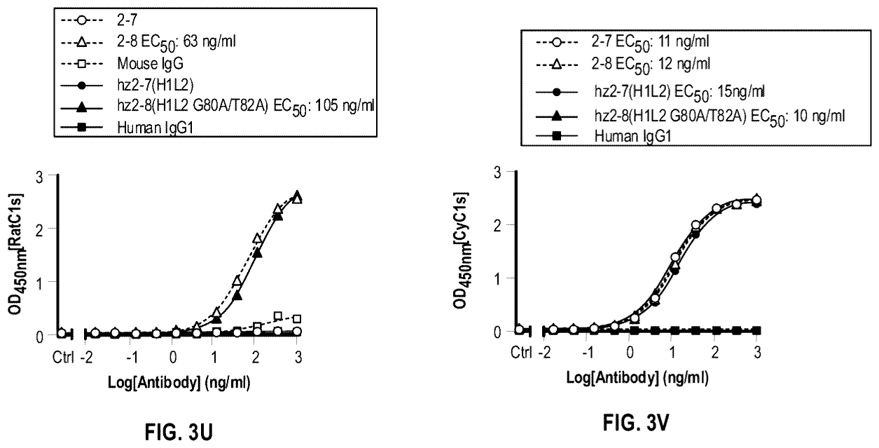

Exemplary anti-C1s antibodies showed high affinity for biologically appropriate targets. Anti-C1s antibodies showed high affinity binding to proenzyme (native) C1s protein and to active C1s protein. As demonstrated by exemplary embodiments of affinity measurements using multiple methods as disclosed Examples 2 and 3 and illustrated in FIGS. 2 and 3, antibodies 2-7, 2-8, hz2-7(H1L2), hz2-8(H1L2), hz2-8(H1L2 G80A), and hz2-8(H1L2 G80A/T82A) exhibited favorable affinity characteristics.

### Anti-C1s Antibodies Having Cross-Reactivity With Non-Human Targets

It is desirable for therapeutically useful antibodies or antibody fragments to have sufficient cross-reactivity with non-human targets (non-human homologues) from sources that would be relevant for further studies such as preclinical efficacy studies, animal models of disease, toxicology studies, etc., such that the antibodies or antibody fragments should recognize, e.g., a rat homologue and/or a primate homologue such as from cynomolgus monkey. As demonstrated by exemplary embodiments in functional screening assays disclosed an Example 2 and illustrated in FIG. 2, antibodies 2-7, 2-8, hz2-7(H1L2), hz2-8(H1L2), hz2-8(H1L2 G80A), and hz2-8(H1L2 G80A/T82A) inhibited IgM-induced deposition of human C4 from human serum and IgM-induced deposition of cynomolgus monkey C4 from cynomolgus monkey serum, providing antibodies raised against human C1s that have functional cross-reactivity with cynomolgus monkey C1s in a cynomolgus monkey system. As demonstrated by exemplary embodiments of binding affinity measurements disclosed in Example 3 and illustrated in FIG. 3, antibodies 2-7, 2-8, hz2-7(H1L2), hz2-8(H1L2), and hz2-8(H1L2 G80A/T82A) show detectable binding and high affinity for human C1s and cynomolgus monkey C1s, while antibodies 2-8 and hz2-8(H1L2 G80A/T82A) also showed detectable binding and reasonably high affinity for rat C1s.

### Anti-C1s Antibodies Capable of Selectively Inhibiting the Classical Pathway (CP)

For CP-mediated disorders, for example CP-mediated disorders triggered by complement-fixing, autoantibodies bound to antigens, it is desirable to selectively inhibit the CP upstream of a step where the CP generates common complement system effectors, in particular C3 convertase. Selectively inhibiting the CP is understood to inhibit effects of antibody-antigen complex triggered CP activation while leaving the alternative and lectin complement pathways intact to combat infection. For CP-mediated disorders, selectively inhibiting the CP upstream of the step of C3 convertase generation is understood to selectively inhibit unwanted effects of CP activation associated with the disorder, while leaving the alternative and lectin complement pathways largely intact to combat infection. Anti-C1s antibodies of the invention have selective effects within the CP by inhibiting C1s (i.e., inhibiting at least one C1s activity or function), thereby acting upstream of the formation of common complement system effectors.

As demonstrated by exemplary embodiments disclosed in Example 2, anti-C1s antibodies disclosed herein are capable of binding to C1s and inhibiting C1s functions involved in IgM-induced C4 deposition from human serum and cynomolgus monkey serum, where it is understood inhibition of C4 deposition in the test assay indicates at least one of the following: C4 cleavage does not occur; C3 convertase formation is inhibited (prevented); and/or CP activation does not lead to downstream effects mediated by the common complement pathway effector C3. In particular, anti-C1s antibodies disclosed herein are capable of specifically binding to C1s and inhibiting at least one activity or function of C1s, such that IgM-induced C4 deposition from human serum and cynomolgus monkey serum is inhibited, C4 cleavage is inhibited, C3 convertase formation is inhibited (prevented), and CP activation is inhibited.

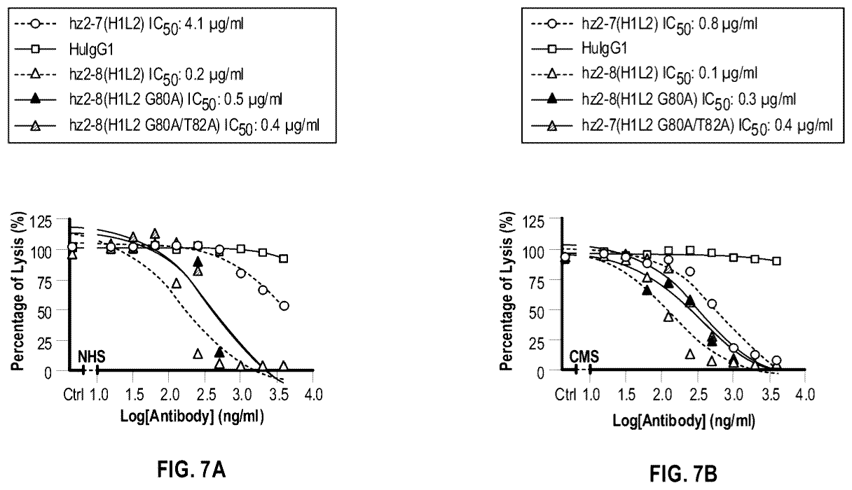

Exemplary embodiments disclosed in Example 7 demonstrate that anti-C1s antibodies disclosed herein are capable of modulating C1s functions involved in the complement classical pathway (CP). Sheep red blood cells coated with rabbit anti-sheep erythrocyte antiserum (hemolysin), also known as antibody-sensitized sheep erythrocytes (“EA” or “EA cells”), have traditionally been used to assay the activity of the complement classical pathway (CP) in serum samples. Commercially available EA cells (Complement Technology, #B202) and known assay systems were used to study complement-mediated lysis of EA in response to exposure to serum. Humanized anti-C1s antibodies hz2-7(H1L2), hz2-8(H1L2), hz2-8(H1L2 G80A), and hz2-8(H1L2 G80A/T82A) inhibited complement-mediated lysis of EA in a dose-dependent manner (Example 7, FIGS. 7A and 7B), where antibody hz2-8(H1L2) was the most potent inhibitor, hz2-8(H1L2), antibodies hz2-8(H1L2 G80A) and hz2-8(H1L2 G80A/T82A) were also highly effective inhibitors, and antibody hz2-7(H1L2) was less effective and showed species dependence.

Anti-C1s antibodies disclosed herein show different levels of inhibition under different conditions, e.g., using different antibodies under the same assay conditions, or using the same antibody under different assay conditions such as using serum from different species. Thus, anti-C1s antibodies disclosed herein provide tools and paths for investigating the roles of specific epitopes and binding interactions in the CP pathway.

### Compositions

Compositions are provided that comprise the present anti-C1s antibody and pharmaceutically acceptable carrier(s) or excipient(s) suitable for the intended use(s) of each composition. Such carriers include but are not limited to: saline, buffer, glucose, water, glycerol, ethanol, excipient, stabilizer, preservative, or combinations thereof. It is understood that the pharmaceutical preparation should match the administration mode.

Anti-C1s antibodies disclosed herein can be administered by any suitable means, including but not limited to injection or parenteral administration. Parenteral administration can include intramuscular, intravenous, intraarterial, intraperitoneal, subcutaneous, intraspinal (including epidural or intrathecal), intraocular, intracerebral, intracerebroventricular, intracardiac, intradermic/intradermal, intraarticular, intralymphatic, or intraosseus administration. Anti-C1s antibodies disclosed herein can be formulated in compositions for introduction into the circulatory system by parenteral administration, in particular intravenous or intraarterial administration. Anti-C1s antibodies disclosed herein can be administered using a device, or as a depot, or in a sustained-release preparations (e.g., semipermeable matrices of solid hydrophobic polymers containing, the antibody, or microcapsules) to allow slow and/or measured and or localized delivery. Anti-C1s antibodies disclosed herein can be formulated and administered using colloidal drug delivery systems (for example, liposomes, albumin microspheres, microemulsions, nano-particles and nano-capsules) or in macroemulsions.

### Methods

Methods are provided for treating a complement-mediated disorder using an effective amount of an anti-C1s antibody disclosed herein. Without wishing to be bound by a particular mechanism of action, methods are provided for precise targeting and inhibition of C1s using anti-C1s antibodies disclosed herein, thereby allowing precise targeting of C1s to selectively modulate activity of the CP. Methods for precise targeting and inhibition of C1s can be methods for using routes of administration to target locations or systems involved in a disorder, methods for using timing of administration to target critical periods, and combinations of these methods. Methods are provided for treating a complement-mediated disorder wherein the CP is activated by complement-fixing autoantibodies bound to self-antigens, and wherein this activation of the CP results in deleterious effects on tissues bearing the self-antigen. Methods are provided for treating a disorder related to unwanted activation of the CP, wherein such methods use an effective amount of an anti-C1s antibody disclosed herein to selectively modulate the activity of CP processes such as C4 cleavage and deposition leading to unwanted attack and destruction of self-tissues. In particular, methods are provided for treating autoimmune conditions by inhibiting downstream effects of CP activation by complexes of complement-fixing autoantibodies bound to self-antigens on platelets.

Methods and compositions are provided for precise targeting and inhibition of using routes of administration to target locations or systems involved in a disorder. Without wishing to be bound by a particular mechanism of action, methods and compositions are provided for treating a disorder characterized by unwanted, abnormal, inappropriate, or excessive CP activation in the circulatory system can be targeted by introducing anti-C1s antibodies into the circulatory system, e.g., by intravenous or intraarterial administration. Without wishing to be bound by a particular mechanism of action, methods and compositions are provided for treating a disorder characterized by unwanted, abnormal, inappropriate, or excessive CP activation in the central nervous system (CNS) can be targeted by introducing anti-C1s antibodies into one or more CNS tissues or structures by intraspinal (including epidural or intrathecal), intraocular, intracerebral, or intracerebroventricular administration. Without wishing to be bound by a particular mechanism of action, methods and compositions are provided for treating a disorder characterized by unwanted, abnormal, inappropriate, or excessive CP activation in one or more joints can be targeted by introducing anti-C1s antibodies into one or more joints by intraarticular administration.

Methods are provided to treat immune thrombocytopenic purpura (ITP) by precise targeting of C1s using an effective amount of an anti-C1s antibody disclosed herein. Compositions of anti-C1s antibodies disclosed here can be formulated for introduction into the circulatory system, e.g., for intravenous and/or intraarterial administration, and introduced by methods that allow targeting of and inhibition of C1s in the circulatory system. Without wishing to be bound by a particular mechanism of action, methods using anti-C1s antibodies disclosed herein can inhibit antibody-mediated removal of platelets by inhibiting the CP. Without wishing to be bound by a particular mechanism of action, methods using anti-C1s antibodies disclosed herein can inhibit C4 deposition on platelets that leads to unwanted platelet destruction. Methods provided herein allow precise targeting of C1s when the CP pathway has been activated by complement-fixing autoantibodies bound to self-antigens on platelets, wherein inhibition of C1s activity interrupts the CP pathway and prevents downstream effects including, but not limited to, inhibiting IgM-induced deposition of C4 from serum, inhibiting antibody-antigen complex mediated C4 cleavage; inhibiting antibody-antigen complex mediated deposition of C4b on platelets, platelet destruction, platelet elimination, thrombocytopenia, and bleeding disorders.

Methods are provided to treat immune neuromyelitis optica (NMO), by precise targeting of C1s using an effective amount of an anti-C1s antibody disclosed herein. Compositions of anti-C1s antibodies disclosed here can be formulated for targeted introduction into the CNS, in particular for intraspinal (e.g., epidural or intrathecal), or intracerebral/intracerebroventricular introduction, and introduced by methods that allow targeting of and inhibition of C1s in the CNS where complement-dependent cytotoxicity (CDC) may occur. Without wishing to be bound by a particular mechanism of action, methods using anti-C1s antibodies disclosed herein can inhibit complement-dependent cytotoxicity (CDC) triggered by autoantibody binding to self antigen, including but not limited to inhibiting antibody-antigen complex mediated C4 cleavage; inhibiting antibody-antigen complex mediated deposition of C4b on astrocytes, inhibiting downstream effects of antibody-antigen complex mediated CP activation, and inhibiting downstream inflammation leading to, e.g., oligo-dendrocyte and neuronal injury, and inflammatory demyelinating lesions in the CNS, particularly in the spinal cord and optic nerve.

Methods and compositions provided herein allow precise timing of treatments. Without wishing to be bound by a particular mechanism of action, anti-C1s antibodies disclosed herein can be administered during an acute phase of a disorder, to inhibit CP-mediated processes during an acute phase of the disorder. Without wishing to be bound by a particular mechanism of action, using anti-C1s antibodies to selectively inhibit CP-mediated processes during an acute phase of a disorder may interrupt an acute incident and/or prevent progression of the disorder, and later allow the CP to return to normal activity after anti-C1s antibodies have dissociated from C1s targets.

Methods and compositions provided herein low precise targeting by timing and location of treatments. Without wishing to be bound by a particular mechanism of action, the timing of administration of anti-C1s antibodies disclosed herein can occur prior to (e.g., in response to an indicator of possible onset of an acute phase) or during an acute phase of a disorder to selectively inhibit CP-mediated processes during the acute phase without inhibiting the functioning of other complement pathways, and the route of administration can be selected to target specific tissues, systems, or structures involved in the disorder. Without wishing to be bound by a particular mechanism of action, using anti-C1s antibodies to inhibit CP-mediated processes prior to or during an acute phase of a disorder may prevent or interrupt an acute incident without inhibiting normal functioning of other complement pathways, and later allow the CP to return to normal activity after anti-C1s antibodies have dissociated from C1s targets.

Methods and compositions provided herein allow control of the amount of therapeutic agent present at one or more time points during treatment. Anti-C1s antibodies disclosed herein can inhibit C1s activity, and selectively inhibit CP pathway activity, in a dose-dependent manner. Without wishing to be bound by a particular mechanism of action, the dose sufficient to ameliorate at least one biological effect or symptom of the disorder (the “effective amount”) may depend on factors specific to a subject in need thereof, such that treatment may comprise determining the dose of anti-C1s antibody needed to constitute an effective amount for the subject to be treated and administering that effective amount of air anti-C1s antibody to the subject, where levels of the therapeutic agent (anti-C1 antibody) are expected to decline after treatment due to dissociation from C1s targets and clearance from blood.

Methods for treating a complement-mediated disorder as provided herein comprise administering an effective amount of an anti-C1s antibody disclosed herein to a subject in need thereof, wherein administration of the effective amount of anti-C1s antibody ameliorates at least one biological effect or symptom associated with the disorder. Methods for treating a complement-mediated disorder associated with autoantibody-triggered activation of the CP are provided wherein administration of an effective amount of an anti-C1s antibody disclosed herein to a subject in need thereof, inhibits the CP pathway and prevents downstream effects of unwanted CP activation.

The following examples are offered to illustrate, but not to limit, the claimed invention.

### EXAMPLES

### Example Antibody Production and Identification of Antibodies That Bind C1s

The production of novel monoclonal antibodies against C1s was carried out under contract by GenScript USA, Inc. (Piscataway, NJ), utilizing in vivo rodent immunization and single B cell cloning technology. Active form of C1s protein (Complement Technology Inc., CompTech A104 C1s Enzyme) was used as antigen to immunize BALB/cJ and SJL mice (The Jackson Laboratories). Sufficient plasma titers as determined by an enzyme-linked immunosorbent assay (ELISA) were obtained, which trigged downstream antibody recovery and screening activities. Secondary lymphoid organs were harvested for CD138 plasma cells enrichment, which were then loaded on BEACON® 14K chip (Berkeley Lights, Inc., Emeryville, CA) to screen for single B cells that secrete antibodies specifically bind to C1s antigen. Positive single B cell clones were selected and exported for subsequent variable domain sequencing and recombinant antibody expression. Full-length recombinant chimeric antibodies were obtained by expression in ExpiCHO cells using pcDNA3.4-VH-CH where VH is from a positive B cell clone (mouse) and CH is IgG1 (human), and pcDNA3.4-VL-CL where VL is from a positive B cell clone (mouse) and CL is kappa (human). Recombinantly expressed and purified antibodies were further screened for functional activities in an in vitro cell-free system. From the positive C1s-binding B cell clones that were obtained, 159 clones were successfully exported for antibody sequencing, and 39 clones were sequenced with one cognate pair of heavy chain and light chain. All 39 anti-C1s clones were recombinantly expressed and purified, and subjected to functional screening.

### Example 2. Functional Screening of Anti-C1s Antibodies

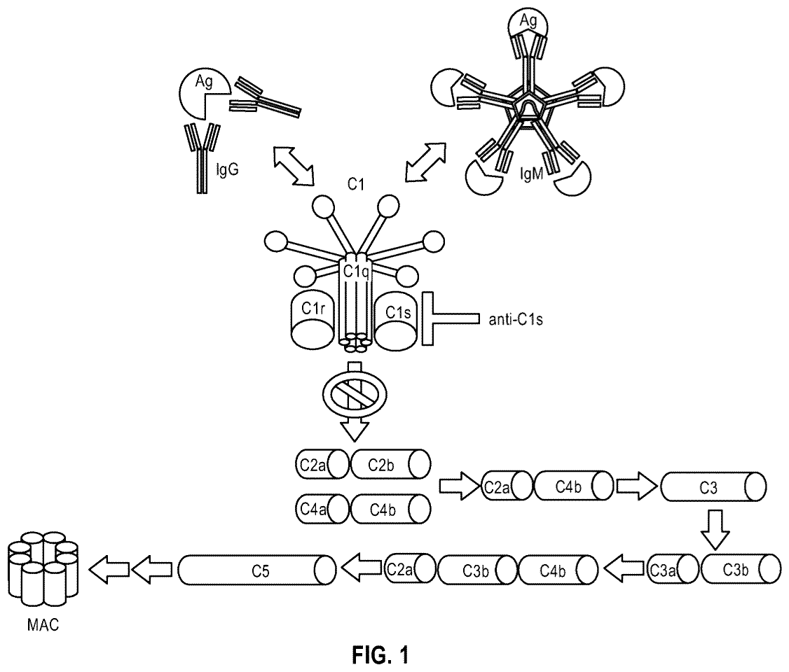

Anti-C1s antibodies from all 39 anti-C1s clones were recombinantly expressed and purified, and then subjected to a functional screening assay to identify anti-C1s antibodies capable of inhibiting IgM-induced C4 deposition. Briefly, anti-C1s antibodies were incubated with serum in IgM-coated plates under conditions suitable for IgM-induced deposition of C4 from the serum, followed by detection of the amount of deposited C4. This functional assay is used as a surrogate assay for C1s activity in accordance with the functional model wherein C4 has to be cleaved by active C1s prior to C4b deposition (in particular, C4b deposition on target cells such as platelets), such that measuring the effects of anti-C1s antibodies on IgM-induced deposition of C4 from serum serves to report the effects on anti-C1s antibodies on C1s activity. This functional assay serves as a surrogate assay for C1s activity (C1s function, C1s biological activity) in antibody-antigen complex mediated CP activation and downstream effects of such CP activation (see, FIG. 1). Anti-C1s antibodies capable of inhibiting IgM-induced C4 deposition were chosen for further investigation.

The functional screening, was carried out by first coating 300 ng/well of IgM from human serum (Sigma-Aldrich I8260) in 96-well plates overnight. The next morning, plates were blocked with gelatin blocking buffer (1% in PBS, Alfa Aesar J62755) for 1 h before adding various concentrations of anti-C1s antibodies diluted in gelatin blocking buffer to each well. After a brief equilibration of anti-C1s antibodies by gentle shaking of the plates, NHS (normal human serum, CompTech NHS) were added to each well to make a final concentration of 1.25% (v/v). The plates were mixed well and incubated at 37° C. for 1 h to allow for C4 deposition induced by IgM. The plates were then washed extensively before incubating with goat anti-human C4 (CompTech A205) diluted in gelatin blocking buffer for 1 h. After incubation, the plates were washed again and incubated with rabbit anti-goat IgG (H+L) secondary antibody HRP conjugated (Invitrogen 81-1620) diluted in gelatin blocking buffer for 1 h. At last, the plates were washed with PBS and color developed with ELISA liquid substrate (Sigma-Aldrich T4444), followed by stopping the reaction with addition of the same volume of ELISA liquid substrate of 1M H₂SO₄. Bound antibody was measured by absorbance at OD_{450 nm} to determine the amount of deposited C4. FIG. 2A shows the results for the top candidates, recombinant antibodies from clone 2-7 (designated anti-C1s antibody 2-7) and recombinant antibodies from clone 2-8 (designated anti-C1s antibody 2-8), as well as the results for antibodies from one of the non-functional clones (3-30) that bind C1s but do not demonstrably inhibit C1s function as measured in the functional assay. The half-maximal inhibitor concentration (IC₅₀) was determined for each antibody that showed an ability to inhibit IgM-induced C4 deposition, with IC₅₀ values for anti-C1s antibody 2.7 and anti-C1s antibody 2-8 between 0.3 and 0.7 μg/ml.

The functional screening assay as described above was repeated using normal cynomolgus monkey serum (CMS, CompTech NCYS). FIG. 2B shows that anti-C1s antibodies 2-7 and 2-8 effectively inhibited IgM-induced deposition of cynomolgus monkey C4 as well, where anti-C1s antibody 2-7 has an IC₅₀ of approximately 0.3 μg/ml, and anti-C1s antibody 2-8 has an IC₅₀ of approximately 0.5 μg/ml.

### Generation and Screening of Humanized Anti-C1s Antibody Variants

Humanization of the parental antibody was performed utilizing CDR grafting onto human antibody frameworks. Homology modeling of the parental antibody's 3-dimensional structure was first performed to establish a structural model of the parental antibody. Amino acid sequences for the variable fragment framework were identified based on the overall sequence identity, matching VH-VL interface positions, similarly classed CDR canonical positions (Kabat numbering), and removal of potential N-glycosylation sites. Humanized antibodies were designed by creating multiple hybrid sequences that fuse selected parts of the parental antibody sequence with the human framework sequences. The isotypes chosen to format humanized antibody were IgG1 for the heavy chain and IgG1 kappa for the light chain. Using the 3D model, these humanized sequences were methodically analyzed by eye and computer modeling to isolate the sequences that would most likely retain antigen binding. The goal was to maximize the amount of human sequence in the final humanized antibodies while retaining the original antibody specificity. Humanized variants, pairing the humanized VH and VL were then expressed using a pcDNA3.4-VL-CL-IRES-VH-CH vector in ExpiCHO cells, where IRES is “internal ribosome entry site” which allows expression of light chain and heavy chain simultaneously, were designed and constructed. Expressed recombinant humanized variants were then purified for affinity analysis.

In one round of designing, generating, and testing variants as part of an affinity analysis, three VH variants were generated with the VH-CDRs of the parental antibody in corresponding positions in three different human IgG1-derived frameworks, and three VL (VK) variants were generated with the VL-CDRs of the parental antibody in corresponding positions in three different human IgG1 kappa-derived frameworks. A total of nine (9) humanized variants representing every combination of the VH and VL (VK) variants were prepared according to a 3 VH×3 VK matrix, evaluated for antigen binding characteristics (kₒₙ, k_{off}, KD).

Variants that showed desired antigen binding affinity based on cutoff values were selected for further evaluation and development. In some cases, parental CDR sequences were modified to avoid potential unwanted events such as aspartate isomerization or asparagine deamidation.

Humanized anti-C1s antibodies hz2-7(H1L2) and hz2-8(H1L2) were chosen from among all the humanized variants tested, because of their superior capabilities of inhibiting IgM-induced C4 deposition from NHS and CMS (see FIGS. 2C and 2D). Comparison of IC₅₀ values shows that antibody hz2-7(H1L2) inhibited IgM-induced C4 deposition from NHS (FIG. 2D) and CMS (FIG. 2C) less effectively than its parental antibody 2-7 (FIG. 2A (NHS). FIG. 2B (CMS)), but antibody hz2-8(H1L2) inhibited IgM-induced C4 deposition from NHS (FIG. 2C) and CMS (FIG. 2D) more effectively than its parental antibody 2-8 (FIG. 2A (NHS), FIG. 2B (CMS)).

FIG. 2C shows results for inhibition of IgM-induced C4 deposition from NHS by humanized antibodies hz2-7(H1L2) and hz2-8(H1L2), and variants hz2-7(H1L2 G131A), hz2-8(H1L2 G80A), and hz2-8(H1L2 G80A/T82A). The following IC₅₀ values were calculated for C1s-binding antibodies capable of inhibiting IgM-induced C4 deposition from NHS (FIG. 2C): IC₅₀ for hz2-7(H1L2) is 1.1 μg/ml; IC₅₀ for hz2-8(H1L2) is 0.2 μg/ml; IC₅₀ for hz2-8(H1L2 G80A) is 0.5 μg/ml; and IC₅₀ for hz2-8(H1L2 G80A/T82A) is 0.4 μg/ml. As shown in FIG. 2C, variant hz2-7(H1L2 G131A) became functionally inactive after the mutation G131A was introduced, which is located within CDR3, so no IC₅₀ value was calculated for functionally inactive variant hz2-7(H1L2 G131A). Variants hz2-8(H1L2 G80A) and hz2-8(H1L2 G80A/T82A) were both functionally active although both were less effective than hz2-8(H1L2) (FIG. 2C).

FIG. 2D shows results for inhibition of IgM-induced C4 deposition from CMS by humanized antibodies hz2-7(H1L2) and hz2-8(H1L2), and variants hz2-7(H1L2 G131A), hz2-8(H1L2 G180A) and hz2-8(H1L2 G80A/T82A). Comparison of FIG. 2C and FIG. 2D shows that the same pattern of results was obtained when CMS (FIG. 2D) was utilized instead of NHS (FIG. 2C). The following IC₅₀ values were calculated for C1s-binding antibodies capable of inhibiting IgM-induced C4 deposition from CMS (FIG. 2D): IC₅₀ for hz2-7(H1L2) is 0.9 μg/ml; IC₅₀ for hz2-8(H1L2) is 0.2 μg/ml; IC₅₀ for hz2-8(H1L2 G80A) is 0.5 μg/ml; and IC₅₀ for hz2-8(H1L2 G80A/T82A) is 0.4 μg/ml. No IC₅₀ value was calculated for functionally inactive variant hz2-7(H1L2 G131A).

Antibody hz2-8(H1L2 G80A/T82A), which has both aspartate isomerization and asparagine deamidation sites removed, was chosen for further development.

Table 1.a. below shows the SEQ ID NOs assigned to the heavy chain (HC) and light chain (LC) of each full-length antibody used in the Examples, and presents the VH amino acid sequence and SEQ NO: and VL amino acid sequence and SEQ ID NO: for each antibody, with CDRs indicated by underlining.

TABLE 1
Anti-C1s Antibodies
Table 1.a. Variable Regions of Anti-C1s Antibodies
Anti-C1s antibody 2-7 (HC SEQ ID NO: 71 and LC SEQ ID NO: 73)
HC variable region (VH) of 2-7 (CDRs indicated by underlining):
MYLGLNCVFIVFLLKGVQSEVKLEESGGGLVPPGGSMKLSCVASGFTFSNYFMNWVR
QSPEKGLEWVAEIRLKFTNYATHYAESVEGRFTISRDDSKSSVYLQMNNLRAEDTGIYY
CTRDYGSRNGYFDYWGQGTTLTVSS (SEQ ID NO: 1)
LC variable region (VL) of 2-7 (CDRs indicated by underlining):
MRCLAEFLGLLVLWIPGAIGDIVMTQAAPSVPVTPGESVSISCRSSKSLLHSNGNTFLYW
FLQRPGQSPQLLIYRMSNLASGVPDRFSGSGSGTAFTLRVSRVEAEDVGIYYCMQHLEY
PYTFGGGTKLEIK (SEQ ID NO: 6)
Anti-C1s antibody 2-8 (HC SEQ ID NO: 75 and LC SEQ ID NO: 77)
HC variable region (VH) of 2-8 (CDRs indicated by underlining):
MKCSWVIFFLMAVVTGVNSEVQLQQSGAELVRPGALVKLSCKASGFNIKEYYMHWV
KQRPEQGLEWIGWIDPENGDTIYDPKFQGKASITADTSSNTAYLQLSSLTSEDTAVYYC
ARSRLFFAYWGQGTLVTVSA (SEQ ID NO: 11)
LC variable region (VL) of 2-8 (CDRs indicated by underlining):
MESQIQVLVFVFLWLSGVDGDIVMTQSHKFMSTSVGDRVSFTCKASQDVRTAVDWYQ
QKPGQSPKLLIYSASYRYTGVPDRFTGTGSGTDFTFTISSVQAEDLAVYFCQQQYTTPYT
FGGGTKLEIK (SEQ ID NO: 16)
Anti-C1s antibody hz2-7(H1L2) (HC SEQ ID NO: 79 and LC SEQ ID NO: 81)
HC variable region (VH) of hz2-7(H1L2) (CDRs indicated by underlining):
MDPKGSLSWRILLFLSLAFELSYGEVQLLESGGGLVQPGGSLRLSCAASGFTFSNYFMN
WVRQAPGKGLEWVSEIRLKFTNYATHYADSVKGRFTISRDDSKSTLYLQMNSLRAEDT
AVYYCTRDYGSRNGYFDYWGQGTLVTVSS (SEQ ID NO: 21)
LC variable region (VL) of hz2-7(H1L2) (CDRs indicated by underlining)
METDTLLLWVLLLWVPGSTGDIVMTQTPLSLPVTPGEPASISCRSSKSLLHSNGNTFLY
WFLQKPGQSPQLLIYRMSNLASGVPDRFSGSGSGTDFTLKISRVEAEDVGVYYCMQHL
EYPYTFGGGTKLEIK SEQ ID NO: 26)
Anti-C1s antibody hz2-7(H1L2 G131A) (HC SEQ ID NO: 83 and LC SEQ ID NO: 85)
HC variable region (VH) of hz2-7(H1L2 G131A) (CDRs indicated by underlining;
CDR residues that differ from parental CDR sequence indicated in bold):
MDPKGSLSWRILLFLSLAFELSYGEVQLLESGGGLVQPGGSLRLSCAASGFTFSNYFMN
WVRQAPGKGLEWVSEIRLKFTNYATHYADSVKGRFTISRDDSKSTLYLQMNSLRAEDT
AVYYCTRDYGSRNAYFDYWGQGTLVTVSS (SEQ ID NO: 31)
LC variable region (VL) of hz2-7(H1L2 G131A):
Protein sequence of the VL (CDRs indicated by underlining):
METDTLLLWVLLLWVPGSTGDIVMTQTPLSLPVTPGEPASISCRSSKSLLHSNGNTFLY
WFLQKPGQSPQLLIYRMSNLASGVPDRFSGSGSGTDFTLKISRVEAEDVGVYYCMQHL
EYPYTFGGGTKLEIK (SEQ ID NO: 36)
Anti-C1s antibody hz2-8(H1L2) (HC SEQ ID NO: 87 and LC SEQ ID NO: 89)
HC variable region (VH) of hz2-8(H1L2) (CDRs indicated by underlining):
MDPKGSLSWRILLFLSLAFELSYGQVQLVQSGAEVKKPGASVKVSCKASGFNIKEYYM
HWVRQAPEQGLEWMGWIDPENGDTIYAQKFQGRVTITADTSTNTAYMELSSLRSEDT
AVYYCARSRLFFAYWGQGTLVTVSS (SEQ ID NO: 41)
LC variable region (VL) of hz2-8(H1L2) (CDRs indicated by underlining):
METDTLLLWVLLLWVPGSTGDIQMTQSPKSLSASVGDRVTITCRASQDVRTALDWYQ
QKPGQSPKLLIYSASYRYSGVPSRFSGSGSGTDFTLTISSLQPEDFATYYCQQQYTTPYTF
GQGTKLEIK (SEQ ID NO: 46)
Anti-C1s antibody hz2-8 (H1L2 G80A/T82A) (HC SEQ ID NO: 91 and LC SEQ ID
NO: 93)
HC variable region (VH) of hz2-8 (H1L2 G80A/T82A) (CDRs indicated by underlining:
CDR residues that differ from parental CDR sequence indicated in bold):
MDPKGSLSWRILLFLSLAFELSYGQVQLVQSGAEVKKPGASVKVSCKASGFNIKEYYM
HWVRQAPEQGLEWMGWIDPENADAIYAQKFQGRVTITADTSTNTAYMELSSLRSEDT
AVYYCARSRLFFAYWGQGTLVTVSS (SEQ ID NO: 51)
LC variable region (VL) of hz2-8 (H1L2 G80A/T82A (CDRs indicated by underlining):
METDTLLLWVLLLWVPGSTGDIQMTQSPKSLSASVGDRVTITCRASQDVRTALDWYQ
QKPGQSPKLLIYSASYRYSGVPSRFSGSGSGTDFTLTISSLQPEDFATYYCQQQYTTPYTF
GQGTKLEIK (SEQ ID NO: 56)
Anti-C1s antibody hz2-8 (H1L2 G80A) (HC SEQ ID NO: 95 and LC SEQ ID NO: 97)
HC variable region (VH) of hz2-8 (H1L2 G80A) (CDRs indicated by underlining: CDR
residues that differ from parental CDR sequence indicated in bold):
MDPKGSLSWRILLFLSLAFELSYGQVQLVQSGAEVKKPGASVKVSCKASGFNIKEYYM
HWVRQAPEQGLEWMGWIDPENADTIYAQKFQGRVTITADTSTNTAYMELSSLRSEDT
AVYYCARSRLFFAYWGQGTLVTVSS (SEQ ID NO: 61)
LC variable region (VL) of hz2-8 (H1L2 G80A) (CDRs indicated by underlining):
METDTLLLWVLLLWVPGSTGDIQMTQSPKSLSASVGDRVTITCRASQDVRTALDWYQ
QKPGQSPKLLIYSASYRYSGVPSRFSGSGSGTDFTLTISSLQPEDFATYYCQQQYTTPYTF
GQGTKLEIK (SEQ ID NO: 66)
Table 1.b. Consensus CDR sequences
Consensus CDR sequences by alignment of: Antibody 2-7; hz2-7(HL2); and hz2-7(h1l2
(G131A)
Consensus HC CDR-1: GFTFSNYFMN (SEQ ID NO: 129)
Consensus HC CDR-2: EIRLKFTNYATHYA(E/D)SV(E/K)G (SEQ ID NO: 130)
Consensus HC CDR-3: DYGSRN(G/A)YFDY (SEQ ID NO: 131)
Consensus LC CDR-1: RSSKSLLHSNGNTFLY (SEQ ID NO: 132)
Consensus LC DCR-2: RMSNLAS (SEQ ID NO: 133)
Consensus LC CDR-3: MQHLEYPYT (SEQ ID NO: 134)
Consensus CDR sequences by alignment of: Antibody 2-8; hz2-8(HIL2); hz2-8(HIL2
G80A/T82A); and hz2-8(HIL2 G80A)
Consensus HC CDR-1: GFNIKEYYMH (SEQ ID NO: 135)
Consensus HC CDR-2: WIDPEN(G/A)D(T/A)IY(D/A)(P/Q)KFQG(SEQ ID NO: 136)
Consensus HC CDR-3: SRLFFAY (SEQ ID NO: 137)
Consensus LC CDR-1: (K/R)ASQDVRTA(V/L)D (SEQ ID NO: 138)
Consensus LC CDR-2: SASYRY(T/S) (SEQ ID NO: 139)
Consensus LC CDR-1: QQQYTTPYT (SEQ ID NO: 140)

### Example 3. Binding Affinity of Anti-C1s Antibodies

### Anti-C1s Antibody Affinity and Binding Kinetics: Measurements Using Bio-Layer Interferometry

No-Layer Interferometry technology was used for anti-C1s is antibody affinity measurement and binding kinetics determinations with OCTET® RED96e system (Sartorius AG). Pre-hydrated Anti-Ruman IgG Fc Capture (AHC) biosensors were first equilibrated in 1×KB (Kinetic Buffer, 1×PBS pH 7.4+0.02% Tween-20+0.1% BSA) for 120 see for the first baseline, followed by loading with 10 mg/ml anti-C1s antibody (2-7, FIG. 3A; 2-8, FIG. 3B; hz2-7(H1L2), FIG. 3C; hz2-8(H1L2 G80A/T82A, FIG. 3D) onto AHC biosensors for 240 sec. Then, the second baseline signal was established for 120 sec before association with various concentrations of active human C1s (CompTech A104) for 240-360 sec. At last, analyte was dissociated in 1×KB for 360 sec. Anti-C1s antibodies 2-7 and 2-8 were also analyzed against human C1s proenzyme (CompTech A103) (data not shown). Data analysis was carried out using OCTET® Data Analysis HT Software (Sartorius AG). KD, kₒₙ, k_{off} and R² values were calculated for each antibody and target combination, as summarized in FIG. 3E showing high affinity binding with KD values in the low nanomolar to picomolar range. FIG. 3E further shows that anti-C1s antibodies 2-7 and 2-8 gave exactly the same calculated values for C1s proenzyme and active C1s protein, with K_{D} values between 0.14 to 1.2 nM.

### Anti-C1s Antibody Binding Affinity: Measurements Using ELISA

ELISA was carried out by first coating 96-well plates with 200 ng/well of human active C1s (CompTech A104, FIG. 3F) or human C1s proenzyme (CompTech A103, FIG. 3G) proteins overnight. The next morning, plates were blocked with SuperBlock T20 blocking buffer (Thermo Fisher Scientific Inc., Catalog No. 37536) for 1 h with gentle shaking. Then, various concentrations of anti-C1s antibodies diluted in SuperBlock T20 blocking buffer were added to the plates and incubated for 1 h with gentle shaking. Human IgG1 (BioXCell BP0297) was used as a negative control. The plates were washed before incubating with HRP conjugated secondary antibodies diluted in SuperBlock T20 blocking buffer (goat anti-human IgG antibody, HRP conjugate (Millipore AP309P)) for 1 h with gentle shaking. At last, the plates were washed and color developed with ELISA liquid substrate (Sigma-Aldrich), followed by stopping the reaction with addition of the same volume of ELISA liquid substrate of 1M H₂SO₄. Bound antibody was measured by absorbance at OD_{450 nm}.

FIG. 3F and FIG. 3G together demonstrate that top candidates 2-7 and 2-8, and their humanized variants hz2-7(H1L2) and hz2-8(H1L2 G80A/T82A), respectively, all showed strong binding to human active C1s (CompTech A104) (FIG. 3F) and human C1s proenzyme (CompTech A103) (FIG. 3G), with half maximal effective concentration (EC₅₀) values between 8 to 13 ng/ml. The EC₅₀ for each antibody and target combination was determined as follows: for antibody 2-7, EC₅₀ for human active C1s is 12 ng/ml and EC₅₀ for human C1s proenzyme is 9 ng/ml; for antibody 2-8, EC₅₀ for human active C1s is 12 ng/ml and EC₅₀ for human C1s proenzyme is 10 ng/ml; for variant hz2-7(H1L2), EC₅₀ for human active C1s is 13 ng/ml and EC₅₀ for human C1s proenzyme is 12 ng/ml; and for variant hz2-8(H1L2 G80A/T82A), EC₅₀ for human active C1s is 10 ng/ml and EC₅₀ for human C1s proenzyme is 8 ng/ml. Control assays using human IgG1 at the same concentrations showed that these patterns did not reflect nonspecific binding (FIG. 3F-3G).

### Anti-C1s Antibody Cross-Reactivity: Measurements Using Bio-Layer Interferometry

Pre-hydrated Anti-Human IgG Fc Capture (AHC) biosensors were first equilibrated in 1×KB (Kinetic Buffer, 1×PBS pH 7.4+0.02% Tween-20+0.1% BSA) for 120 sec for the first baseline, followed by loading with 10 mg/ml anti-C1s antibody (2-7, FIGS. 3H&N; 2-8, FIGS. 3I, L&O: hz2-7(H1L2), FIGS. 3J&P; hz2-8(H1L2 G80A/T82A, FIGS. 3K, M&Q) onto AHC biosensors for 240 sec. Then, the second baseline signal was established for 120 sec before association with various concentrations of human C1s (produced in house (SEQ ID NO: 99) shown in FIGS. 3H-K), rat C1s (produced in house (SEQ ID NO:105) shown in FIGS. 3L-M) or cynomolgus monkey C1s (produced in house (SEQ ID NO: 103) shown in FIGS. 3N-Q) for 240-360 sec. At last, analyte was dissociated in 1×KB for 360 sec. Data analysis was done using OCTET® Data Analysis HT Software (Sartorius AG). KD, kₒₙ, k_{off} and R² values were calculated for each antibody and target combination, as summarized in FIG. 3R. Top candidates 2-7 and 2-8, and their humanized variants hz2-7(H1L2) and hz2-8(H1L2 G80A/T82A), respectively, all showed strong binding to human C1s and cynomolgus monkey C1s, but no binding to mouse C1s (FIG. 3R) with K_{D} between 0.012 to 3.5 nM. Anti-C1s antibody 2-7 and its humanized variant hz2-7 (H1L2) showed no binding to rat C1s, while 2-8 and its humanized variant bz2-8(H1L2 G80A/T82A) showed binding to rat C1s with K_{D} values between 16 to 17 nM (FIG. 3R). The binding affinity of 2-8 and hz2-8(H1L2 G80A/T82A) for rat C1s was weaker than the binding affinity for human C1s and cynomolgus monkey C1s (FIG. 3R).

### Anti-C1s Antibody Cross-Reactivity: Measurements Using ELISA

ELISA was carried out first by coating 96-well plates with 200 ng/well of human C1s (identified as full-length human C1s, HuC1s, wt human C1s) (produced in house (SEQ ID NO: 99), FIG. 3S), mouse C1s (produced in house (SEQ ID NO: 101), FIG. 3T), rat C1s (produced in house (SEQ ID NO: 105), FIG. 3U) or cynomolgus monkey C1s (produced in house (SEQ ID NO: 103), FIG. 3V) proteins overnight. The next morning, plates were blocked with SuperBlock T20 blocking buffer (Thermo Scientific 37536) for 1 h with gentle shaking. Then, various concentrations of anti-C1s antibodies diluted in SuperBlock T20 blocking buffer were added to the plates and incubated for 1 h with gentle shaking. Mouse IgG (Sigma-Aldrich I5381) and Human IgG1 (BioXCell BP0297) were used as negative controls. The plates were washed before incubating with HRP conjugated secondary antibodies diluted in SuperBlock T20 blocking buffer (goat anti-mouse IgG (H+L) cross-adsorbed secondary antibody HRP (Invitrogen G-21040) or goat anti-human IgG antibody, HRP conjugate (Millipore AP309P)) for 1 h with gentle shaking. At last, the plates were washed and color developed with ELISA liquid substrate (Sigma-Aldrich), followed by stopping the reaction with addition of the same volume of ELISA liquid substrate of 1M H₂SO₄. Bound antibody was measured by absorbance. at OD_{430 nm}. Top candidates 2-7 and 2-8, and their humanized variants hz2-7(H1L2) and hz2-8(H1L2 G80A/T82A), respectively, all showed strong binding to human C1s (FIG. 3S) and cynomolgus monkey C1s (FIG. 3V), but no binding to mouse C1s (FIG. 3T) with EC₅₀ between 4 to 15 mg/ml. Anti-C1s 2-7 and its humanized variant hz2-7(H1L2) showed no binding to rat C1s, while 2-8 and its humanized variant hz2-8(H1L2 G80A/T82A) showed weaker binding to rat C1s (FIG. 3U) with EC₅₀ between 63 to 105 ng/ml.

### Example 4. Bending of Anti-C1s Antibodies to Full-Length and Truncated C1s Measured by ELISA

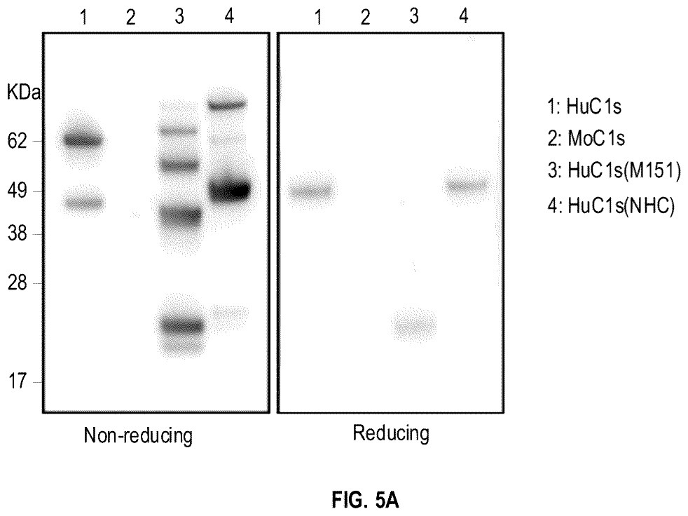

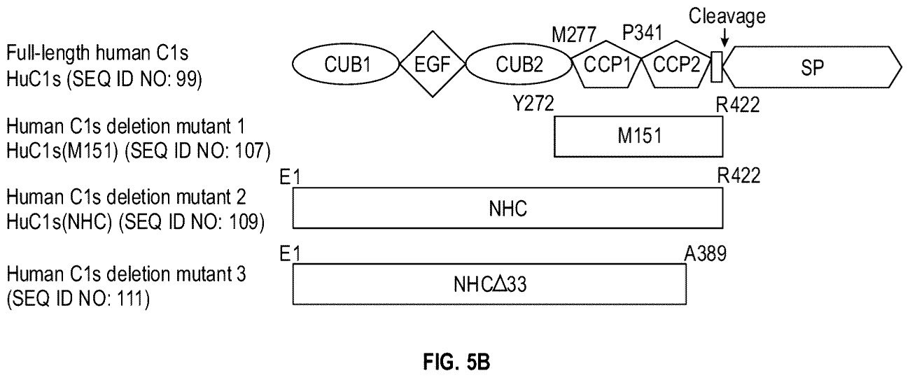

Binding of anti-C1s antibodies to a full-length human C1s protein, a full-length mouse C1s protein, an NHC fragment of human C1s protein (FIG. 5B for alignment), and a middle fragment of human C1s located within the NHC (identified as “M151” or “huC1sM151”), encompassing 151 amino acids at residues 272 to 422 of human C1s, upstream of auto cleavage site R422-I423 (FIG. 5B for alignment), was measured by ELISA. Constructs were developed for expression of full length human C1s (HuC1s, or “wt human C1s”) (SEQ ID NO: 99), full length mouse C1s (moC1s) (SEQ ID NO: 101), human C1s deletion mutant 1 (huC1sM151) (SEQ ID NO: 107), and human C1s deletion mutant 2 corresponding to the NHC of C1s (huC1sNHC) (SEQ ID NO: 109), with a C-terminal 6xHis tag on each expressed protein. All expression and purification of full-length and truncated C1s constructs (SEQ ID NOS: 100, 102, 108, 110) and resulting proteins (SEQ ID NOS: 99, 101, 107, 109) was performed in house.

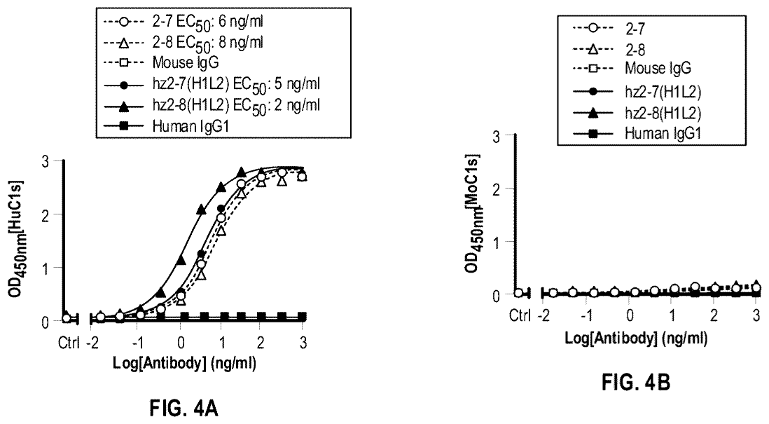

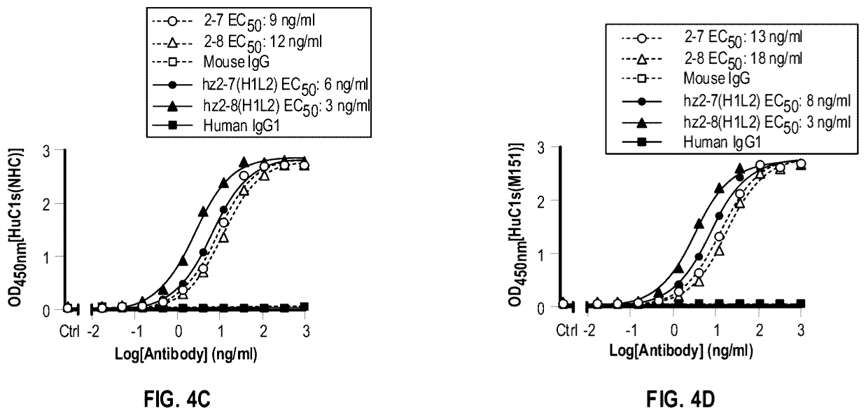

ELISA was carried out by first coating 96-well plates with 200 ng/well of full-length or truncated C1s proteins overnight. The next morning, plates were blocked with SuperBlock120 blocking buffer (Thermo Scientific 37536) for 1 h with gentle shaking. Then, various concentrations of anti-C1s antibodies diluted in SuperBlock T20 blocking buffer were added to the plates and incubated for 1 h with gentle shaking. Mouse IgG (Sigma-Aldrich I5381) and human IgG1 (BioXCell BP0297) were used as negative controls. The plates were washed before incubating with HRP conjugated secondary antibodies diluted in SuperBlock T20 blocking buffer (goat anti-mouse IgG3 (H+L) cross-adsorbed secondary antibody HRP (Invitrogen G-21040) or goat anti-human IgG antibody, HRP conjugate (Millipore AP309P)) for 1 h with gentle shaking. At last, the plates were washed and color developed with ELISA liquid HRP substrate (Sigma-Aldrich), followed by stopping the reaction with addition of 1M H₂SO₄ in the same volume as the ELISA liquid substrate. Bound antibody was measured by absorbance at OD_{450 nm}. Results are shown in FIG. 4. Top candidates 2-7 and 2-8, and their humanized variants hz2-7(H1L2) and hz2-8(H1L2), respectively, all showed strong binding to fall length human C1s (FIG. 4A) but no binding to full-length mouse C1s (FIG. 4B). All anti-C1s antibodies showed binding to both truncated human C1s (NHC and M151) with higher EC₅₀ compared to full-length C1s (FIGS. 4C-D), indicating that human C1s (M151) preserved the major binding sites for anti-C1s, and the flanking regions may facilitate holding the right (or preferred) conformation.

### Example 5. Bending of Anti-C1s Antibodies to Full-Length and Truncated C1s Demonstrated by Western Blot

Western blot analysis was first carried out by resolving 200 ng/lane full-length or truncated C1s (human C1s, mouse C1s, NHC of human C1s and M151 of human C1) on SDS-PAGE with or without reducing agent (Invitrogen B0004), as follows: 200 ng full-length human C1s (HuC1s; SEQ ID NO:99) in lane 1, 200 ng full length mouse C1s (MoC1s: SEQ ID NO: 101) in lane 2, 200 ng truncated human C1s M151 (HuC1s(M151); SEQ ID NO: 107) in lane 3, and 200 ng truncated human C1s NHC (HuC1s(NHC); SEQ ID NO: 109) in lane 4. The proteins were later transferred to nitrocellulose membranes, and blocked with 5% blotto (ChemCruz sc-2325) diluted in TBST (Teknova T1688) overnight. The next morning, the blocked membranes were first incubated with 1 μg/ml anti-C1s antibody diluted in 5% blotto-TBST for 1 h with gentle shaking. Then, the membranes were washed and incubated with HRP conjugated secondary antibodies diluted in 5% blotto-TBST (goat anti-human IgG antibody, HRP conjugate (Millipore AP309P)) for 1 h with gentle shaking. At last, membranes were washed extensively, and developed using SuperSignal West Dura Extended Duration Substrate (Thermo Scientific 34075) and imaged under azure biosystems. Representative results are drown in FIG. 5A presenting results for anti-C1s antibody 2-8,

Where the blot on the left is from the gel running under non-reducing conditions, and the blot on the right is from the gel running under reducing conditions. On the blot of the gel running without reducing, agent (left), lane 1 shows antibody 2-8 binding to two (2) major bands of full-length human C1s (HuC1s) with the top band corresponding to the full-length HuC1s and the bottom band corresponding to the auto-cleaved heavy chain, lane 2 shows no detectable antibody 2-8 to full length mouse C1s (MoC1s), lane 3 shows antibody 2-8 binding to multiple bands of truncated human C1s M151 (HuC1s(M151)), and lane 4 shows antibody 2-8 binding to a few higher molecular weight bands of truncated human C1s NHC (HuC1s(NHC)). On the blot of the gel running with reducing agent (right), lane 1 shows a low level of antibody 2-8 binding to a band corresponding to the auto-cleaved heavy chain of the full-length human C1s (HuC1s), lane 2 shows no detectable antibody 2-8 to full length mouse C1s (MoC1s), lane 3 shows a low level of antibody 2-8 binding to the monomer of truncated human C1s M151 (HuC1s(M151)), and lane 4 shows a low level of antibody 2-8 binding to the monomer of truncated human C1s NHC (HuC1s(NHC)).

Results using anti-C1s antibodies 2-7, hz2-7(H1L2), and hz2-8(H1L2)) showed the same binding pattern (data not shown) as the results for antibody 2-8 shown in FIG. 5A. These results showed that the binding site for anti-C1s antibody 2-7, anti-C1s antibody 2-8, anti-C1s antibody hz2-7(H1L2), and anti-C1s antibody hz2-8(H1L2) is within the human C1s M151 region on the NHC.

### Example 6. Epitome Mapping of Anti-C1s Antibodies Using Truncated C1s and C1s With Point Mutations

As indicated in Examples 4-5, the epitopes of anti-C1s antibodies disclosed herein are located within human C1s (M151) (SEQ ID NO: 107). Human C1s (M151) is the last 151 amino acids upstream of auto-cleavage site (R422-I423) within the human C1s NHC. In order to further define the epitopes within human C1s (M151), a further truncated C1s mutant was designed. This mutant is named human C1s deletion mutant 3 (NHCΔ33) (SEQ ID NO:111) and is lacking the last 33 amino acids, corresponding to G390-R422 fragment of human C1s (FIG. 5B for alignment). C1s (NHCΔ33) (SEQ ID NO:111) was cloned and expressed with a C-terminal 6xHis tag, and its expression and purification were performed in house. FIG. 5B shows a schematic comparison and alignment of full-length human C1s, HuC1s (SEQ ID NO: 99), human C1s deletion mutant 1 (M151), HuC1s(M151), (SEQ ID NO: 107), human deletion mutant 2 (NHC), HuC1s(NHC) (SEQ ID NO: 109) and human C1s deletion mutant 3 (NHCΔ33), human C1s (NHCΔ33) (SEQ ID NO:111) used in these these experiments.

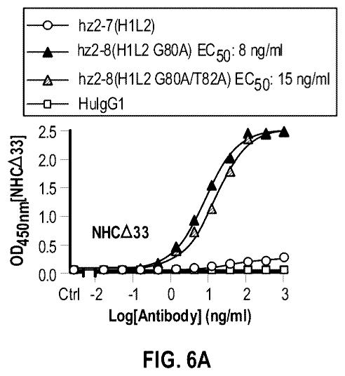

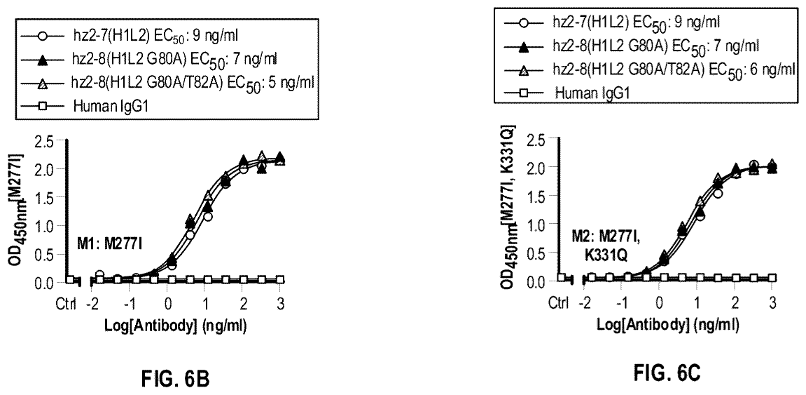

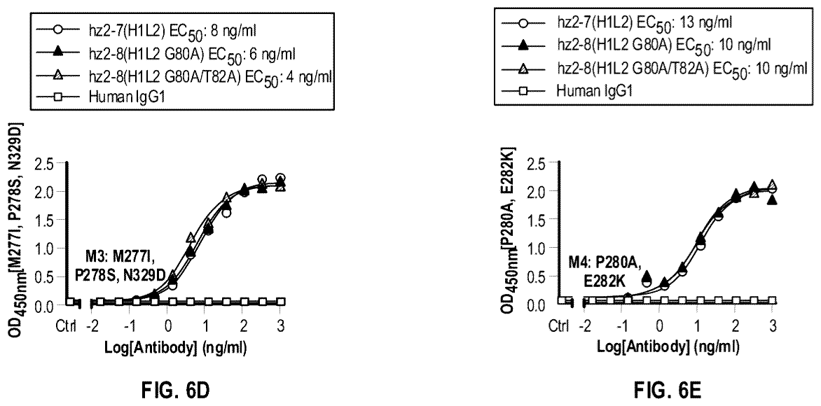

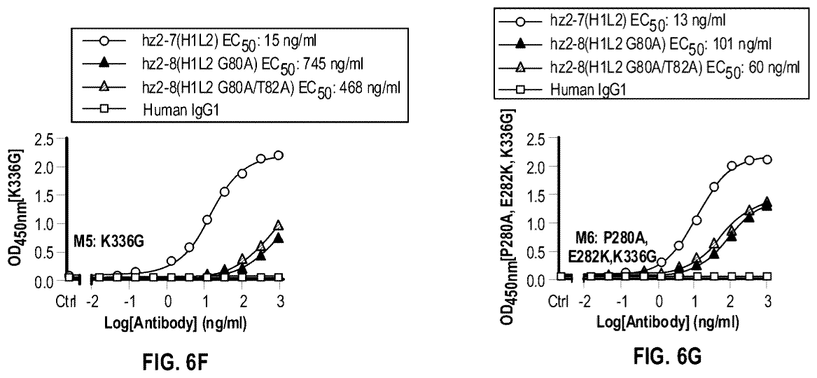

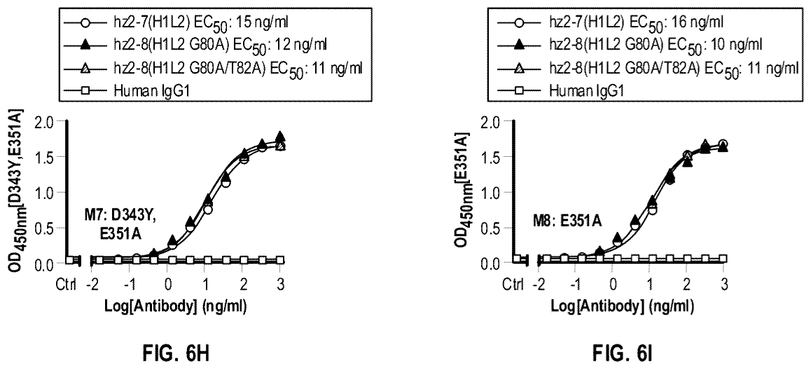

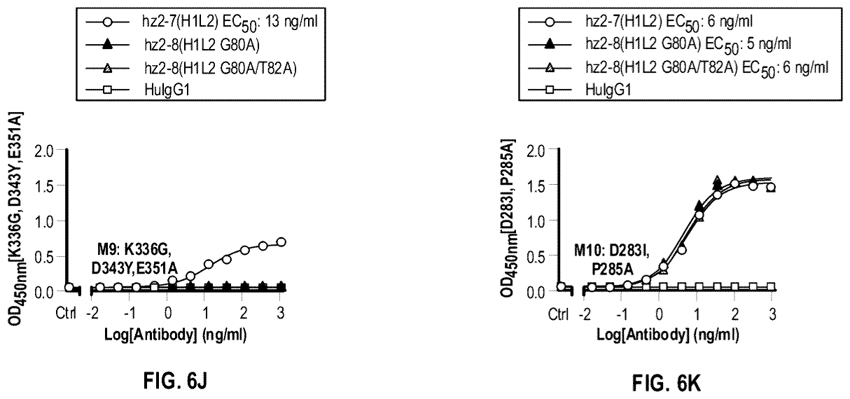

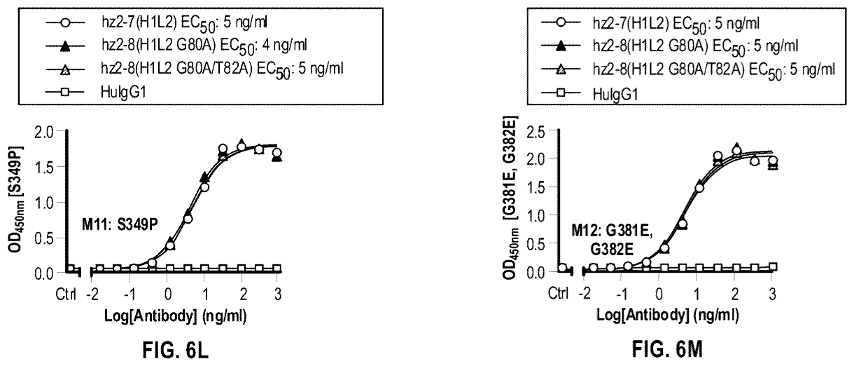

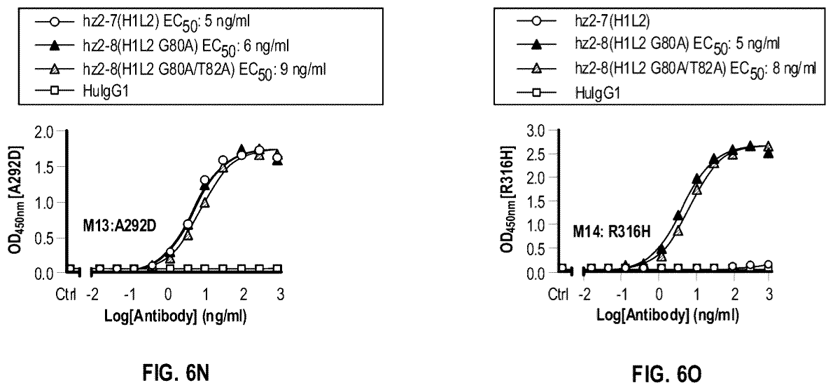

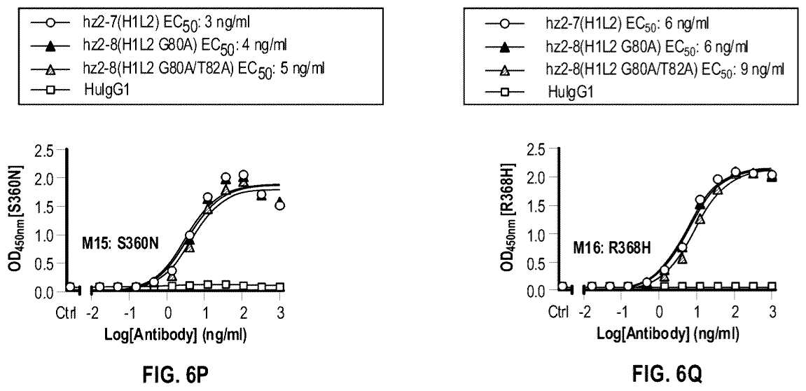

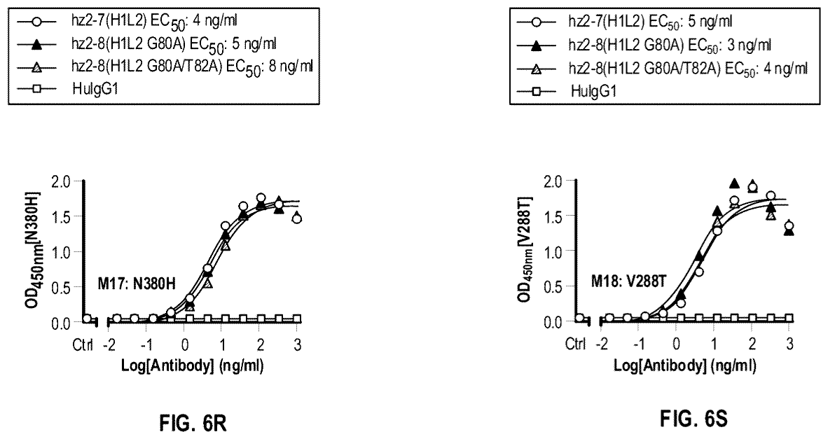

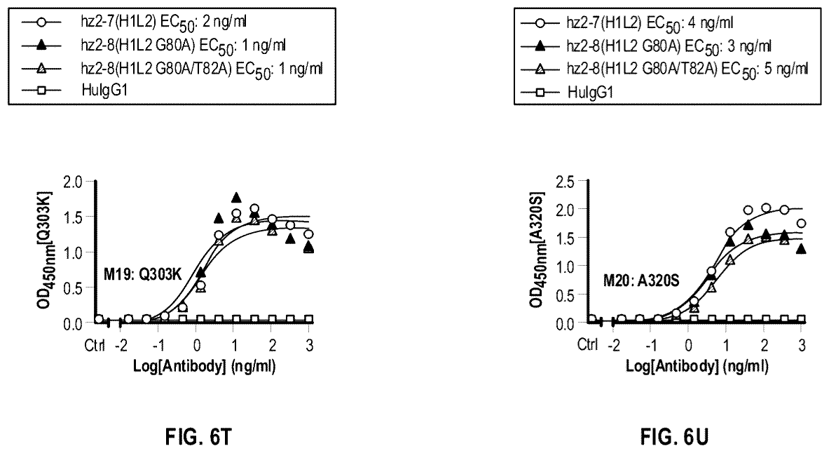

Binding of various anti-C1s antibodies to human C1s (NHCΔ33) (SEQ ID NO: 111) was carried out by ELISA as described in Example 4, and results are shown in FIG. 6A. Hz2-7(H1L2) showed almost no binding to human C1s (NHCΔ33), while hz2-8(H1L2 G80A) and hz2-8(H1L2 G80A/T82A) showed strong binding to C1s (NHCΔ33) with EC₅₀ values of 8 ng/ml and 15 ng/ml, respectively. As expected, human IgG1 showed no binding. Such distinct binding profiles of humanized 2-7 variant versus humanized 2-8 variants strongly suggest that antibody 2-7 and 2-8 bind to distinct epitopes within the human C1 (M151) as demonstrated in Example 4. The results indicate that the epitopes of hz2-7(H1L2) are within the 33 amino acids located upstream of cleavage sites (G390-R422), and the epitopes of hz2-8(H1L2 G380A) and hz2-8(H1L2 G80A/T82A) are within the rest of M151 (Y272-A389).

In order to identify the epitopes of hz2-8(H1L2 G80A) and hz2-8(H1L2 G80A/T82A), we introduced various point mutations to human C1s either individually or in double/triple combinations, and obtained 20 human C1s mutants named M1-M20 (SEQ ID NOs: 112-128 and 141-143) as shown in Table 2.

For all point mutations, the residue at a certain position of human C1s was substituted for the corresponding residue of mouse C1s. Among 22 residues that were mutated, 6 are identical between human C1s and rat C1s, 10 are identical between rat C1s and mouse C1s, and the remaining 6 are unique residues at the corresponding positions for human-, rat-, and mouse C1s. M1-M20 of human C1s (SEQ ID NOS: 112-128 and 141-143) were cloned with a C-terminal 6xHis tag, expressed and purified in house.

TABLE 2
Point mutations of human C1s and corresponding
residues of rat- and mouse C1s

| | | | | C1s mutants |
|---|---|---|---|---|
| Point | Human | Rat | Mouse | with point |
| mutation | C1s | C1s | C1s | mutation |
| M277I | M | I | | M1 (SEQ ID |
| | | | | NO: 112), M2 |
| | | | | (SEQ ID NO: |
| | | | | 113), M3 (SEQ |
| | | | | ID NO: 114) |
| P278S | P | | S | M3 (SEQ ID |
| | | | | NO: 114) |
| P280A | P | | A | M4 (SEQ ID |
| | | | | NO: 115), M6 |
| | | | | (SEQ ID NO: |
| | | | | 117) |
| E282K | E | | K | M4 (SEQ ID |
| | | | | NO: 115), M6 |
| | | | | (SEQ ID NO: |
| | | | | 117) |
| D283I | D | I | | M10 (SEQ ID |
| | | | | NO: 121) |
| P285A | P | A | | M10 (SEQ ID |
| | | | | NO: 121) |
| V288T | V | I | T | M18 (SEQ ID |
| | | | | NO: 141) |
| A292D | A | E | D | M13 (SEQ ID |
| | | | | NO: 124) |
| Q303K | Q | K | | M19 (SEQ ID |
| | | | | NO: 142) |
| R316H | R | N | H | M14 (SEQ ID |
| | | | | NO: 125) |
| A320S | A | S | | M20 (SEQ ID |
| | | | | NO: 143) |
| N329D | N | | D | M3 (SEQ ID |
| | | | | NO: 114) |
| K331Q | K | Q | | M2 (SEQ ID |
| | | | | NO: 113) |
| K336G | K | R | G | M5 (SEQ ID |
| | | | | NO: 116), M6 |
| | | | | (SEQ ID NO: |
| | | | | 117), M9 (SEQ |
| | | | | ID NO: 120) |
| D343Y | D | | Y | M7 (SEQ ID |
| | | | | NO; 118), M9 |
| | | | | (SEQ ID NO: |
| | | | | 120) |
| S349P | S | P | | M11 (SEQ ID |
| | | | | NO: 122) |
| E351A | E | | A | M7 (SEQ ID |
| | | | | NO: 118), M8 |
| | | | | (SEQ ID NO: |
| | | | | 119), M9 (SEQ |
| | | | | ID NO: 120) |
| S360N | S | D | N | M15 (SEQ ID |
| | | | | NO: 126) |
| R368H | R | H | | M16 (SEQ ID |
| | | | | NO: 127) |
| N380H | N | Q | H | M17 (SEQ ID |
| | | | | NO: 128) |

| | | | |
|---|---|---|---|
| G381E | G | E | M12 (SEQ ID |
| | | | NO: 123) |
| G382E | G | E | M12 (SEQ ID |
| | | | NO: 123) |

Binding of anti-C1s antibodies to mutants M1-M20 of human C1s was measured by ELISA as described in Example 4, and results are shown in FIGS. 6B-6U. All anti-C1s humanized variants including hz2-7(H1L2), hz2-8(H1L2 G80A) and hz2-8(H1L2 G80A1/T82A) maintained strong binding properties to the majority (16 out of 20) of C1s mutants including M1 (FIG. 6B), M2 (FIG. 6C), M3 (FIG. 6D), M4 (FIG. 6E), M7 (FIG. 6H), M8 (FIG. 6I), M10 (FIG. 6K), M11 (FIG. 6L), M12 (FIG. 6M), M13 (FIG. 6N), M15 (FIG. 6P), M16 (FIG. 6O), M17 (FIG. 6R), M18 (FIG. 6S), M19 (FIG. 6T) and M20 (FIG. 6U). Isotype control human IgG1 showed binding to none of the mutants. These results strongly suggest that 20 out of the 22 point mutations, i.e. M277I, P2785, P280A, E282K, D283I, P28A, V288T, A292D, Q303K, A320S, N329D, K331Q, D343Y, S349P, E351A, S360N, R368H, N380H, G381E and G382E, had no effect on the binding of hz2-7(H1L2), hz2-8(H1L2 G80A) and hz2-8(H1L2 G80A/T82A) to the C1s. Therefore, the 20 residues are NOT critical binding sites for any of the anti-C1s antibodies examined.

As shown in FIG. 6A, binding sites of hz2-7(H1L2) are located within the last 33 amino acids of C1s NHC (G390-R422). M1-M20 of human C1s were not expected to affect binding of hz2-7(H1L2) because none of the point mutations is located within this region. Surprisingly, M14 of human C1s (SEQ ID NO: 125), which only contains a single point mutation of R316H, completely abolished binding of hz2-7(H1L2) while it had no effect on binding of hz2-8(H1L2 G80A) or hz2-8(H1L2 G80A/T82A) (FIG. 6O). Human IgG1 showed no binding to M14 as expected (FIG. 6O). These results strongly suggest that in addition to G390-R422, R316 is another critical residue involved in the binding of hz2-7(H1L2), further confirmed previous indications that the binding sites of hz2-7(H1L2) are different from that of hz2-8(H1L2 G80A) and hz2-8(H1L2 G80A/T82A).

Furthermore, as shown in FIG. 6J, mutant M9 (SEQ ID NO: 120) encompassing point mutations of K336G, D343Y and E351A, completely eliminated binding of hz2-8(H1L2 G80A) and hz2-8(H1L2 G80A/T82A), while hz2-7(H1L2) retains binding with high potency. As previously demonstrated, D343Y and E351A are NOT important for binding of any anti-C1s antibodies examined (FIG. 6H-I). Therefore, K336 is a (or possibly the) critical residue for binding of hz2-8(H1L2 G80A) and hz2-8(H1L2 G80A/T82A). In addition to M9, the K336G point mutation is also present in M5 and M6 of human C1s, either individually (M5) or in combination with other non-essential residues (M6). As shown in FIG. 6F-G, K336G single mutation resulted in a significantly reduced binding of hz2-8(H1L2 G80A) and hz2-8(H1L2 G80A/T82A) but had no effect on hz2-7(H1L2), confirmed that K336 is the key residue for binding of hz2-8(H1L2 G80A) and hz2-8(H1L2 G80A/T82A) to C1s, constituting a unique epitope for 2-8 antibody.

Taken together, the epitope of hz2-7(H1L2) comprises R316 and G390-R422, and the critical residue for binding of hz2-8((H1L2 G80A) and hey-8(H1L2 G80A/T82A) to C1s is K336. The. results indicated that epitopes of antibody 2-7 versus antibody 2-8 are distinct although all occur within the middle 151 amino acid region of human C1s (M151).

### Example 7. Inhibition by Anti-C1s Antibodies of Complement-Mediated Lyses of Antibody-Sensitized Sheep Erythrocyte

Sheep red blood cells coater with rabbit anti-sheep erythrocyte antiserum (hemolysin), which is also known as antibody-sensitized sheep erythrocytes (EA), have traditionally been used to assay the activity of the complement classical pathway in serum samples, where these assays allow determination of the CH50 titer of the serum. Taking advantage of commercially available EA (Complement Technology, #B202), inhibition of complement-mediated lysis of EA by anti-C1s antibodies was assessed. Briefly, humanized anti-C1s variants, including hz2-7(H1L2), hz2-8(H1L2), hz2-8(H1L2G80A) and hz2-8(H1L2 G80A/T82A), were first serially diluted in GVB++ buffer (Complement Technology, Catalog #B100, manufacturer ingredient list: 0.1% gelatin, 5 mM Veronal, 145 mM NaCl, 0.025% NaN₃, 0.15 mM calcium chloride, 0.5 mM magnesium chloride, pH 7.3). The diluted anti-C1s antibodies were then mixed with normal human serum (NHS, FIG. 7A) or with cynomolgus monkey serum (CMS, FIG. 7B) to reach a final serum concentration of 1%. Antibody and serum mixture were equilibrated at room temperature before adding EA cells. For each reaction, approximately 0.5-1×10⁸ EA cells were used. To determine the 0% lysis background control value, EA cells were mixed with buffer only (“no serum controls”). To determine the 100% lysis control value. GVB++ buffer was replaced with water. EA cells were gently mixed with the antibody and serum, then the mixtures were incubated at 37° C. for 1 h with periodic mixing. After incubation, the remaining EA cells were spun down, and the absorbance of supernatant were determined by measuring OD_{540 nm}. To calculate the percentage of lysis that occurred wider specific incubation conditions, the value of the 0% lysis background was first subtracted from each absorbance reading, and the results were then normalized to the 100% lysis control (with 0% lysis background subtracted) to determine the percentage of lysis that occurred.

The results for human complement inhibition by anti-C1s antibody are shown in FIG. 7A. Antibody hz2-8(H1L2) was the most potent inhibitor of complement-mediated lysis of EA, with IC₅₀ of 0.2 μg/ml. Antibodies hz2-8(H1L2 G80A) and hz2-8(H1L2 G80A1/T82A), each of which are sequence-optimized variants of hz2-8(H1L2), also effectively inhibited human complement-mediated lysis of EA with similar IC₅₀ between 0.4-0.5 μg/ml. hz2-7(H1L2) was the weakest inhibitor in this case with IC₅₀ of 4.1 μg/ml, and the maximal inhibition only reached about 50% at the highest concentration tested (4 μg/ml). The results for cynomolgus monkey complement inhibition as shown in FIG. 7B are similar to the human serum results, where hz2-8(H1L2) was the most potent inhibitor of cynomolgus monkey complement-mediated lysis of EA with an IC₅₀ of 0.1 μg/ml, and its variants hz2-8(H1L2 G80A) and hz2-8(H1L2 G80A/T82A) also maintained high inhibition potency with similar IC₅₀ values between 0.3-0.4 μg/ml. Hz2-7(H1L2) inhibited cynomolgus monkey complement more effectively than human complement, with IC₅₀ of 0.8 μg/ml and an extent of inhibition that reaches maximum (FIG. 7B). These results strongly support the role of various anti-C1s antibodies in the inhibition of classical complement pathway, which is consistent with findings from Example 2.

While the present invention has been described with reference to certain specific embodiments thereof, it should be understood by those skilled in the art that changes and modifications in the specifically-described embodiments may be carried out without departing from the principles of the present invention, which is intended to be limited only by the scope of the appended claims.

What is claimed is:

1. A recombinant anti-C1s antibody or an antigen-binding fragment thereof that is capable of specifically binding to human C1s and inhibiting C1s, comprising a heavy chain variable region (VH) comprising three heavy chain complementarily determining regions (HC CDRs) and a light chain variable region (VL) comprising three light chain complementarily determining regions (LC CDRs), selected from:
(a) the VH comprising an HC CDR 1 having the amino sequence GFTFSNYFMN (SEQ ID NO: 129, an HC CDR2 having the amino sequence EIRLKFTNYATHYA(E/D)SV(E/K)G (SEQ ID NO: 130), and an HC CDR3 having the amino acid sequence DYGSRN(G/A)YFDY (SEQ ID NO: 131), and the VL comprising an LC CDR1 having the amino acid sequence RSSKSLLHSNGNTFLY (SEQ ID NO: 132), an LC CDR2 having the amino acid sequence RMSNLAS (SEQ ID NO: 133), and an LC CDR3 having the amino acid sequence MQHLEYPYT (SEQ ID NO: 134), and
(b) the VH comprising an HC CDR1 having the amino sequence GFNIKEYYMH (SEQ ID NO: 135), an HC CDR2 having the amino sequence WIDPEN(G/A)D(T/A)IY(D/A)(P/Q)KFQG (SEQ ID NO: 136), and an HC CDR3 having the amino acid sequence SRLFFAY (SEQ ID NO: 137), and the VL comprising an LC CDR1 having the amino acid sequence ASQDVRTA(V/L)D (SEQ ID NO: 138), an LC CDR2 comprising the amino acid sequence SASYRY(T/S) (SEQ ID NO: 139), and LC CDR3 comprising the amino acid sequence QQQYTTPYT (SEQ ID NO: 140).

2. The anti-C1s antibody of claim 1, wherein the antibody is capable of binding to at least one of residues K336, R316, and a region encompassing A390-R422 of human C1s (SEQ ID NO: 99).

3. The anti-C1s antibody of claim 1, wherein the anti-C1s antibody is capable of inhibiting the activity of the classical pathway (CP) of complement activation.

4. The anti-C1s antibody of claim 3, wherein the anti-C1s antibody is capable of at least one of inhibiting downstream effects of C1s activation, inhibiting IgM-induced deposition of C4 from serum, inhibiting antibody-antigen complex mediated C4 cleavage, inhibiting antibody-antigen complex mediated deposition C4b, inhibiting formation of C3 convertase, and inhibiting downstream effects of CP activation.

5. The anti-C1s antibody of claim 1, herein the antibody shows cross-reactivity with at least one non-human C1s.

6. The anti-C1s antibody of claim 5, wherein the non-human C1s is cynomolgus monkey C1s.

7. The anti-C1s antibody of claim 6, wherein the anti-C1s antibody shows cross-reactivity with rat C1s.

8. The anti-C1s antibody of claim 1, wherein the antibody is at least one of a chimeric antibody, a humanized antibody, and an antigen-binding fragment.

9. The anti-C1s antibody of claim 1, comprising at least one polypeptide having an amino acid sequence selected from: SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 11; SEQ ID NO: 12 SEQ ID NO: 13; SEQ ID NO: 14; SEQ ID NO: 16; SEQ ID NO: 17; SEQ ID NO: 18; SEQ ID NO: 19; SEQ ID NO: 21; SEQ ID NO: 22; SEQ ID NO: 23; SEQ ID NO: 24; SEQ ID NO: 26; SEQ ID NO: 27; SEQ ID NO: 28; SEQ ID NO: 29; SEQ ID NO: 31; SEQ ID NO: 32; SEQ ID NO: 33; SEQ ID NO: 34; SEQ ID NO: 36; SEQ ID NO: 37; SEQ ID NO: 38; SEQ ID NO: 39; SEQ ID NO: 41; SEQ ID NO: 42; SEQ ID NO: 43; SEQ ID NO: 44; SEQ ID NO: 46; SEQ ID NO: 47; SEQ ID NO: 48; SEQ ID NO: 49; SEQ ID NO: 51; SEQ ID NO: 52; SEQ ID NO: 53; SEQ ID NO: 54; SEQ ID NO: 56; SEQ ID NO: 57; SEQ ID NO: 58; SEQ ID NO: 59; SEQ NO: 61; SEQ NO: 62; SEQ NO: 63; SEQ NO: 64 SEQ NO: 66: SEQ NO: 67; SEQ ID NO: 68; SEQ NO 69; SEQ NO: 71; SEQ NO: 73; SEQ NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; SEQ ID NO: 95; or SEQ ID NO: 97.

10. The anti-C1s antibody of claim 1, wherein the antibody is one of:
a. an antibody comprising a heavy chain (HC) polypeptide having the amino acid sequence of SEQ ID NO: 71 or a sequence at least about 85% identical to SEQ ID NO: 71, and a light chain (LC) polypeptide having the amino acid sequence of SEQ ID NO: 73 or a sequence at least about 85% identical to SEQ ID NO: 73;
b. an antibody comprising an HC polypeptide having the amino acid sequence of SEQ ID NO: 75 or a sequence at least about 85% identical to SEQ ID NO: 75, and an LC polypeptide having the amino acid sequence of SEQ ID NO: 77 or a sequence at least about 85% identical to SEQ ID NO: 77;
c. an antibody comprising an HC polypeptide having the amino acid sequence of SEQ ID NO: 79 or a sequence at least about 85% identical to SEQ ID NO: 79, and an LC polypeptide having the amino acid sequence of SEQ ID NO: 81 or a sequence at least about 85% identical to SEQ ID NO: 81;
d. an antibody comprising an HC polypeptide having the amino acid sequence of SEQ ID NO: 83 or a sequence at least about 85% identical to SEQ ID NO: 83, and an LC polypeptide having the amino acid sequence of SEQ ID NO: 85 or a sequence at least about 85% identical to SEQ ID NO: 85;
e. an antibody comprising an HC polypeptide having the amino acid sequence of SEQ ID NO: 87 or a sequence at least about 85% identical to SEQ ID NO: 87, and an LC polypeptide having, the amino acid sequence of SEQ ID NO: 89 or a sequence at least about 85% identical to SEQ ID NO: 89;
f. an antibody comprising an HC polypeptide having the amino acid sequence of SEQ ID NO: 91 or a sequence at least about 85% identical to SEQ ID NO: 91, and an LC polypeptide having the amino acid sequence of SEQ ID NO: 93 or a sequence at least about 85% identical to SEQ ID NO: 93; or
g. an antibody comprising an HC polypeptide having the amino acid sequence of SEQ ID NO: 95 or a sequence at least about 85% identical to SEQ ID NO: 95, and an LC polypeptide having the amino acid sequence of SEQ ID NO: 97 or a sequence at least about 85% identical to SEQ ID NO: 97.

11. The anti-C1s antibody of claim 1, wherein the antibody is one of:
a. an antibody comprising a heavy chain variable region (VH) having the amino acid sequence of SEQ ID NO: 1 or a sequence at least about 85% identical to SEQ ID NO: 1, and a light chain variable region (VL) having the amino acid sequence of SEQ ID NO: 6 or a sequence at least about 85% identical to SEQ ID NO: 6;
b. an antibody comprising a VH having the amino acid sequence of SEQ ID NO: 11 or a sequence at least about 85% identical to SEQ ID NO: 11, and an VL having the amino acid sequence of SEQ ID NO: 16 or a sequence at least about 85% identical to SEQ ID NO: 16;
e. an antibody comprising a VH having the amino acid sequence of SEQ ID NO: 21 or a sequence at least about 85% identical to SEQ ID NO: 21, and an VL having the amino acid sequence of SEQ ID NO: 26 or a sequence at least about 85% identical to SEQ ID NO: 26;
d. an antibody comprising a VH having the amino acid sequence of SEQ ID NO: 31 or a sequence at least about 85% identical to SEQ ID NO: 31, and an VL having the amino acid sequence of SEQ ID NO: 36 or a sequence at least about 85% identical to SEQ ID NO: 36;
e. an antibody comprising a VH having the amino acid sequence of SEQ ID NO: 41 or a sequence at least about 85% identical to SEQ ID NO: 41, and an VL having the amino acid sequence of SEQ ID NO: 46 or a sequence at least about 85% identical to SEQ ID NO: 46;
f. an antibody comprising a VH having the amino acid sequence of SEQ ID NO: 51 or a sequence at least about 85% identical to SEQ ID NO: 51, and an VL having the amino acid sequence of SEQ ID NO: 56 or a sequence at least about 85% identical to SEQ ID NO: 56; or
g. an antibody comprising a VH having the amino acid sequence of SEQ ID NO: 61 or a sequence at least about 85% identical to SEQ ID NO: 61, and an VL having the amino acid sequence of SEQ ID NO: 66 or a sequence at least about 85% identical to SEQ ID NO: 66.

12. The anti-C1s antibody of claim 1, wherein the antibody comprises an HC CDR1 of SEQ ID NO: 22, an HC CDR2 of SEQ ID NO: 23, an HC CDR3 of SEQ ID NO: 24, an LC CDR1 of SEQ ID NO: 27, an LC CDR2 of SEQ ID NO: 28, and an LC CDR3 of SEQ ID NO: 29, and further wherein the antibody may comprise 6 or fewer total amino acid substitutions in the CDR regions.

13. The anti-C1s antibody of claim 1, wherein the antibody comprises an HC CDR1 of SEQ ID NO: 52, an HC CDR2 of SEQ ID NO: 53, an HC CDR3 of SEQ ID NO: 54, an LC CDR1 of SEQ ID NO: 57, an LC CDR2 of SEQ ID NO: 58, and an LC CDR3 of SEQ ID NO: 59, and further wherein the antibody may comprise 6 or fewer total amino acid substitutions in the CDR regions.

14. A method of treating a complement-mediated disorder, comprising administering an effective amount of an anti-C1s antibody of claim 1 to a subject in need thereof, wherein administration of the effective amount of anti-C1s antibody inhibits the activity of the CP.

15. The method of claim 14, wherein the complement-mediated disorder is an autoimmune disorder characterized by binding of complement-fixing antibodies to at least one self-antigen.

16. The method of claim 15, wherein the autoimmune disorder is selected from one of immune thrombocytopenic purpura (ITP) and neuromyelitis optica (NMO).

17. The method of claim 14, wherein administration of the effective amount of anti-C1s antibody inhibits at least one of C1s activation, antibody-antigen complex mediated C4 cleavage, and formation of a common complement pathway effector.

18. The method of claim 14, wherein the effective amount of anti-C1s antibody is administered during an acute phase of the disorder.

19. A pharmaceutical composition comprising the anti-C1s antibody of claim 1 and a suitable carrier and/or excipient.

20. An isolated nucleic acid molecule that encodes at least a portion of the anti-C1s antibody of claim 1, wherein the nucleic acid comprises at least one nucleotide sequence selected from
a. an antibody comprising a heavy chain variable region (VH) having the amino acid sequence a heavy-chain (HC) nucleotide sequence selected from: a nucleotide sequence comprising the sequence of SEQ ID NO: 5, a nucleotide sequence comprising a sequence at least about 85% identical to SEQ ID NO: 5, a nucleotide sequence comprising the sequence of SEQ ID NO: 15, a nucleotide sequence comprising a sequence at least about 85% identical to SEQ ID NO: 15, a nucleotide sequence comprising the sequence of SEQ ID NO. 25, a nucleotide sequence comprising a sequence at least about 85% identical to SEQ ID NO: 25, a nucleotide sequence comprising the sequence of SEQ ID NO: 35, a nucleotide sequence comprising a sequence at least about 85% identical to SEQ ID NO: 35, a nucleotide sequence comprising the sequence of SEQ ID NO: 45, a nucleotide sequence comprising a sequence at least about 85% identical to SEQ ID NO: 45, a nucleotide sequence comprising the sequence of SEQ ID NO: 55, a nucleotide sequence comprising a sequence at least about 85% identical to SEQ ID NO: 55, a nucleotide sequence comprising the sequence of SEQ ID NO: 65, and a nucleotide sequence comprising a sequence at least about 85% identical to SEQ ID NO: 65, and
b. a light chain (LC) nucleotide sequence selected from: a nucleotide sequence comprising the sequence of SEQ ID NO: 10, a nucleotide sequence comprising a sequence at least about 85% identical to SEQ ID NO: 10, a nucleotide sequence comprising the sequence of SEQ ID NO: 20, a nucleotide sequence comprising a sequence at least about 85% identical to SEQ ID NO: 20, a nucleotide sequence comprising the sequence of SEQ ID NO. 30, a nucleotide sequence comprising a sequence at least about 85% identical to SEQ ID NO: 30, a nucleotide sequence comprising the sequence of SEQ ID NO: 40, a nucleotide sequence comprising a sequence at least about 85% identical to SEQ ID NO: 40, a nucleotide sequence comprising the sequence of SEQ ID NO: 50, a nucleotide sequence comprising a sequence at least about 85% identical to SEQ ID NO: 50, a nucleotide sequence comprising the sequence of SEQ ID NO: 60, a nucleotide sequence comprising a sequence at least about 85% identical to SEQ ID NO: 60 a nucleotide sequence comprising the sequence of SEQ ID NO: 70, and a nucleotide sequence comprising a sequence at least about 85% identical to SEQ ID NO: 70.

21. A vector comprising a nucleic acid molecule of claim 20.

22. A host cell comprising the vector of claim 21.
